(19) United States
(12) Patent Application Publication
(10) Pub. No.: US 2021/0196731 A1
(43) Pub. Date: Jul. 1, 2021

(54) COMPOSITIONS AND METHODS FOR TREATING OCULAR INFLAMMATION AND OCULAR SCARRING

(71) Applicants: James Jacob Bruvall Armstrong, London (CA); Cindy Mary-Lynn Hutnik, London (CA)

(72) Inventors: James Jacob Bruvall Armstrong, London (CA); Cindy Mary-Lynn Hutnik, London (CA)

(21) Appl. No.: 17/059,766

(22) PCT Filed: May 28, 2019
(86) PCT No.: PCT/CA2019/050724

§ 371 Date: Nov. 30, 2020

Related U.S. Application Data
(60) Provisional application No. 62/677,284, filed on May 29, 2018.

(51) Int. Cl. A61K 31/616; A61P 27/02; A61K 31/25; A61K 31/7036; A61K 38/17; A61K 35/64; A61K 45/06

(52) U.S. Cl. CPC A61K 31/616; A61P 27/02; A61K 31/25; A61K 45/06; A61K 38/1709; A61K 35/64; A61K 31/7036

(57) ABSTRACT

The present disclosure provides compositions and methods for treating ocular inflammation and ocular fibrosis and/or scarring using a cyclooxygenase 2 serine 516 acetylating agent alone or in combination with a cytosolic phospholipase A2 agonist.

### CROSS-REFERENCE TO RELATED APPLICATIONS

The present disclosure claims priority to U.S. Provisional Patent Application No. 62/677,284 filed on May 29, 2018, which is incorporated herein by reference as if set forth in its entirety.

### FIELD OF THE DISCLOSURE

The present disclosure relates generally to treatment of ocular conditions. More particularly, the disclosure relates to compositions and methods for treating ocular inflammation and ocular fibrosis and/or scarring in a subject.

### BACKGROUND OF THE DISCLOSURE

An excessive inflammatory response has long been known to lead to negative outcomes in many ocular surgeries such as glaucoma filtration surgery [1-8], cataract surgery [8-12], pterygium removal [13, 14], and excision of fibrovascular retinal membranes [15-18]. Immediately after ocular surgery, local inflammation promotes the transdifferentiation of fibroblasts into myofibroblasts [19], which then act to remodel the extracellular matrix [20], produce scarring, disrupt tissue functionality [21] and ultimately destroy the delicate ocular anatomy that healthy vision is dependent on [8]. Subconjunctival scarring in association with increased numbers of macrophages, lymphocytes, myofibroblasts and fibroblasts have been associated with a greater risk for glaucoma surgery failure, highlighting the importance of inflammation-induced scarring/fibrosis in the outcome of the procedure [22]. In cataract surgery, inflammation has been associated with several adverse outcomes due to disruption of the blood ocular barrier and influx of inflammatory signalling molecules [23-25]. These adverse outcomes include worsened visual outcomes, corneal edema, elevated intraocular pressure (IOP), pain, posterior capsular opacification and surgically induced miosis [8-12, 26-30]. Treatment with non-steroidal anti-inflammatory drugs (NSAIDs) following cataract surgery has been associated with reduced ocular inflammation and pain [8-12, 26-30]. Similarly, inflammation and myofibroblast activity play a significant role in the development of proliferative vitreoretinopathy following retinal detachment repair surgery, for which corticosteroid anti-inflammatory treatment has been suggested [15-18, 31, 32]. In addition to its impact on ophthalmic surgical outcomes, inflammation induced myofibroblast transdifferentiation and activity are central to the pathophysiology of several significant ocular diseases such as, corneal fibrosis/opacification/neovascularization [33-37], orbital fibrosis (Graves ophthalmopathy) [8, 38, 39], primary open angle glaucoma [8, 13, 40], and the sub-macular fibrosis that occurs in patients with age-related macular degeneration who are refractory to anti-VEGF therapy [8, 41-45]. This highlights the broad impact of ocular inflammation and myofibroblast transdifferentiation on several key ophthalmic pathologies and surgical procedures.

Presently, the first-line therapy for treating inflammation-induced ocular fibrosis and/or scarring following ocular surgery is mitomycin C (MMC). MMC is a cytotoxic agent that is also commonly used for chemotherapeutic treatment of various tumors. Due to the inherent toxicity of MMC, however, its use following ocular surgery is associated with undesirable side effects and complications. Thus, there is an unmet need in the art for safe and effective therapies that mitigate and/or prevent ocular inflammation, fibrosis and/or scarring in subjects.

### SUMMARY OF THE DISCLOSURE

The inventors have invented compositions and methods for decreasing ocular inflammation; decreasing ocular fibrosis and/or scarring; decreasing ocular collagen contraction and/or remodelling; and decreasing ocular fibroblast cellular proliferation.

In an aspect of the disclosure, a pharmaceutical composition comprising a cyclooxygenase 2 (COX2) serine (Ser) 516 acetylating agent and a cytosolic phospholipase A2 (cPLA2) agonist is provided.

In an embodiment of the pharmaceutical composition comprising a COX2 Ser516 acetylating agent and a cPLA2 agonist provided herein, the COX2 Ser516 acetylating agent is acetylsalicylic acid (ASA) or a 2-acetoxyphenyl alkyl sulfide.

In an embodiment of the pharmaceutical composition comprising a 2-acetoxyphenyl alkyl sulfide and a cPLA2 agonist provided herein, the 2-acetoxyphenyl alkyl sulfide is o-(acetoxyphenyl)hept-2-ynyl sulfide (APHS).

In an embodiment of the pharmaceutical composition comprising a COX2 Ser516 acetylating agent and a cPLA2 agonist provided herein, the cPLA2 agonist is gentamicin, tobramycin, mastoparan, phospholipase A2 activating protein (PLAP), tetrahydrofurandiol or melittin.

In an embodiment of the pharmaceutical composition comprising a COX2 Ser516 acetylating agent and a cPLA2 agonist provided herein, the pharmaceutical composition further comprises a pharmaceutically acceptable carrier, diluent or excipient.

In an embodiment of the pharmaceutical composition comprising a COX2 Ser516 acetylating agent and a cPLA2 agonist provided herein, the pharmaceutical composition is formulated for ocular administration.

In an aspect of the disclosure, a method of decreasing ocular inflammation in a subject is provided. The method comprises administering to the subject a COX2 Ser516 acetylating agent and a cPLA2 agonist.

In an aspect of the disclosure, a method of decreasing ocular fibrosis and/or scarring in a subject is provided. The method comprises administering to the subject a COX2 Ser516 acetylating agent and a cPLA2 agonist.

In an aspect of the disclosure, a method of decreasing ocular collagen contraction and/or remodelling in a subject is provided. The method comprises administering to the subject a COX2 Ser516 acetylating agent and a cPLA2 agonist.

In an aspect of the disclosure, a method of decreasing ocular fibroblast cellular proliferation in a subject is provided. The method comprises administering to the subject a COX2 Ser516 acetylating agent and a cPLA2 agonist.

In an embodiment of the method of decreasing ocular fibroblast cellular proliferation in a subject by administering to the subject a COX2 Ser516 acetylating agent and a cPLA2 agonist provided herein, the fibroblast is a myofibroblast.

In an embodiment of the method of decreasing ocular fibroblast cellular proliferation in a subject by administering to the subject a COX2 Ser516 acetylating agent and a cPLA2 agonist provided herein, the fibroblast is a human Tenon's capsule fibroblast (HTCF).

In embodiments of the methods of decreasing ocular inflammation, decreasing ocular fibrosis and/or scarring, decreasing ocular collagen contraction and/or remodelling, and decreasing ocular fibroblast cellular proliferation in a subject by administering to the subject a COX2 Ser516 acetylating agent and a cPLA2 agonist provided herein, the ocular inflammation is inflammation of the conjunctiva.

In embodiments of the methods of decreasing ocular inflammation, decreasing ocular fibrosis and/or scarring, decreasing ocular collagen contraction and/or remodelling, and decreasing ocular fibroblast cellular proliferation in a subject by administering to the subject a COX2 Ser516 acetylating agent and a cPLA2 agonist provided herein, the inflammation of the conjunctiva is caused by conjunctivitis.

In embodiments of the methods of decreasing ocular inflammation, decreasing ocular fibrosis and/or scarring, decreasing ocular collagen contraction and/or remodelling, and decreasing ocular fibroblast cellular proliferation in a subject by administering to the subject a COX2 Ser516 acetylating agent and a cPLA2 agonist provided herein, the conjunctivitis is allergic conjunctivitis.

In embodiments of the methods of decreasing ocular inflammation, decreasing ocular fibrosis and/or scarring, decreasing ocular collagen contraction and/or remodelling, and decreasing ocular fibroblast cellular proliferation in a subject by administering to the subject a COX2 Ser516 acetylating agent and a cPLA2 agonist provided herein, the COX2 Ser516 acetylating agent and the cPLA2 agonist are administered before, during and/or after ocular surgery.

In embodiments of the methods of decreasing ocular inflammation, decreasing ocular fibrosis and/or scarring, decreasing ocular collagen contraction and/or remodelling, and decreasing ocular fibroblast cellular proliferation in a subject by administering to the subject a COX2 Ser516 acetylating agent and a cPLA2 agonist before, during and/or after ocular surgery provided herein, the ocular surgery comprises one or more of:

a. manipulation of the conjunctiva and/or Tenons;

b. a conjunctival incision or excision;

c. implantation of a medical device within or around the eye; and/or

d. a corneal incision.

In embodiments of the methods of decreasing ocular inflammation, decreasing ocular fibrosis and/or scarring, decreasing ocular collagen contraction and/or remodelling, and decreasing ocular fibroblast cellular proliferation in a subject by administering to the subject a COX2 Ser516 acetylating agent and a cPLA2 agonist before, during and/or after ocular surgery provided herein, the ocular surgery is micro-invasive glaucoma surgery, glaucoma filtration surgery, cataract surgery, retinal detachment repair surgery, strabismus surgery, vitrectomy, pterygium removal, an excisional biopsy, trauma reconstruction, or implantation of a stent, valve, implant or shunt within or around the eye.

In embodiments of the methods of decreasing ocular inflammation, decreasing ocular fibrosis and/or scarring, decreasing ocular collagen contraction and/or remodelling, and decreasing ocular fibroblast cellular proliferation in a subject by administering to the subject a COX2 Ser516 acetylating agent and a cPLA2 agonist provided herein, the COX2 Ser516 acetylating agent and the cPLA2 agonist are administered sequentially, in either order.

In embodiments of the methods of decreasing ocular inflammation, decreasing ocular fibrosis and/or scarring, decreasing ocular collagen contraction and/or remodelling, and decreasing ocular fibroblast cellular proliferation in a subject by administering to the subject a COX2 Ser516 acetylating agent and a cPLA2 agonist provided herein, the COX2 Ser516 acetylating agent and the cPLA2 agonist are administered simultaneously.

In embodiments of the methods of decreasing ocular inflammation, decreasing ocular fibrosis and/or scarring, decreasing ocular collagen contraction and/or remodelling, and decreasing ocular fibroblast cellular proliferation in a subject by administering to the subject a COX2 Ser516 acetylating agent and a cPLA2 agonist provided herein, the COX2 Ser516 acetylating agent and the cPLA2 agonist are formulated in the same composition.

In embodiments of the methods of decreasing ocular inflammation, decreasing ocular fibrosis and/or scarring, decreasing ocular collagen contraction and/or remodelling, and decreasing ocular fibroblast cellular proliferation in a subject by administering to the subject a COX2 Ser516 acetylating agent and a cPLA2 agonist provided herein, the COX2 Ser516 acetylating agent and the cPLA2 agonist are administered locally.

In embodiments of the methods of decreasing ocular inflammation, decreasing ocular fibrosis and/or scarring, decreasing ocular collagen contraction and/or remodelling, and decreasing ocular fibroblast cellular proliferation in a subject by administering to the subject a COX2 Ser516 acetylating agent and a cPLA2 agonist provided herein, the COX2 Ser516 acetylating agent is ASA or a 2-acetoxyphenyl alkyl sulfide.

In embodiments of the methods of decreasing ocular inflammation, decreasing ocular fibrosis and/or scarring, decreasing ocular collagen contraction and/or remodelling, and decreasing ocular fibroblast cellular proliferation in a subject by administering to the subject a 2-acetoxyphenyl alkyl sulfide and a cPLA2 agonist provided herein, the 2-acetoxyphenyl alkyl sulfide is APHS.

In embodiments of the methods of decreasing ocular inflammation, decreasing ocular fibrosis and/or scarring, decreasing ocular collagen contraction and/or remodelling, and decreasing ocular fibroblast cellular proliferation in a subject by administering to the subject a COX2 Ser516 acetylating agent and a cPLA2 agonist provided herein, the cPLA2 agonist is gentamicin, tobramycin, mastoparan, PLAP, tetrahydrofurandiol or melittin.

In an aspect of the disclosure, a method of resolving ocular inflammation in a subject is provided. The method comprises administering a COX2 Ser516 acetylating agent to the subject.

In an aspect of the disclosure, a method of decreasing ocular fibrosis and/or scarring in a subject is provided. The method comprises administering a COX2 Ser516 acetylating agent to the subject.

In an aspect of the disclosure, a method of decreasing ocular collagen contraction and/or remodelling in a subject is provided. The method comprises administering a COX2 Ser516 acetylating agent to the subject.

In an aspect of the disclosure, a method of decreasing ocular fibroblast cellular proliferation in a subject is provided. The method comprises administering a COX2 Ser516 acetylating agent to the subject.

In an embodiment of the method of decreasing ocular fibroblast cellular proliferation in a subject by administering a COX2 Ser516 acetylating agent to the subject provided herein, the fibroblast is a myofibroblast.

In an embodiment of the method of decreasing ocular fibroblast cellular proliferation in a subject by administering a COX2 Ser516 acetylating agent to the subject provided herein, the fibroblast is a human Tenon's capsule fibroblast (HTCF).

In embodiments of the methods of decreasing ocular inflammation, decreasing ocular fibrosis and/or scarring, decreasing ocular collagen contraction and/or remodelling, and decreasing ocular fibroblast cellular proliferation in a subject by administering a COX2 Ser516 acetylating agent to the subject provided herein, the ocular inflammation is inflammation of the conjunctiva.

In embodiments of the methods of decreasing ocular inflammation, decreasing ocular fibrosis and/or scarring, decreasing ocular collagen contraction and/or remodelling, and decreasing ocular fibroblast cellular proliferation in a subject by administering a COX2 Ser516 acetylating agent to the subject provided herein, the inflammation of the conjunctiva is caused by conjunctivitis.

In embodiments of the methods of decreasing ocular inflammation, decreasing ocular fibrosis and/or scarring, decreasing ocular collagen contraction and/or remodelling, and decreasing ocular fibroblast cellular proliferation in a subject by administering a COX2 Ser516 acetylating agent to the subject provided herein, the conjunctivitis is allergic conjunctivitis.

In embodiments of the methods of decreasing ocular inflammation, decreasing ocular fibrosis and/or scarring, decreasing ocular collagen contraction and/or remodelling, and decreasing ocular fibroblast cellular proliferation in a subject by administering a COX2 Ser516 acetylating agent to the subject provided herein, the COX2 Ser516 acetylating agent is administered before, during and/or after ocular surgery.

In embodiments of the methods of decreasing ocular inflammation, decreasing ocular fibrosis and/or scarring, decreasing ocular collagen contraction and/or remodelling, and decreasing ocular fibroblast cellular proliferation in a subject by administering a COX2 Ser516 acetylating agent to the subject before, during and/or after ocular surgery provided herein, the ocular surgery comprises one or more of:

a. manipulation of the conjunctiva and/or Tenons;

b. a conjunctival incision or excision;

c. implantation of a medical device within or around the eye; and/or

d. a corneal incision.

In embodiments of the methods of decreasing ocular inflammation, decreasing ocular fibrosis and/or scarring, decreasing ocular collagen contraction and/or remodelling, and decreasing ocular fibroblast cellular proliferation in a subject by administering a COX2 Ser516 acetylating agent to the subject before, during and/or after ocular surgery provided herein, the ocular surgery is micro-invasive glaucoma surgery, glaucoma filtration surgery, cataract surgery, retinal detachment repair surgery, strabismus surgery, vitrectomy, pterygium removal, an excisional biopsy, trauma reconstruction, or implantation of a stent, valve, implant or shunt within or around the eye.

In embodiments of the methods of decreasing ocular inflammation, decreasing ocular fibrosis and/or scarring, decreasing ocular collagen contraction and/or remodelling, and decreasing ocular fibroblast cellular proliferation in a subject by administering a COX2 Ser516 acetylating agent to the subject provided herein, the COX2 Ser516 acetylating agent is ASA or a 2-acetoxyphenyl alkyl sulfide.

In embodiments of the methods of decreasing ocular inflammation, decreasing ocular fibrosis and/or scarring, decreasing ocular collagen contraction and/or remodelling, and decreasing ocular fibroblast cellular proliferation in a subject by administering a 2-acetoxyphenyl alkyl sulfide to the subject provided herein, the 2-acetoxyphenyl alkyl sulfide is APHS.

In embodiments of the methods of decreasing ocular inflammation, decreasing ocular fibrosis and/or scarring, decreasing ocular collagen contraction and/or remodelling, and decreasing ocular fibroblast cellular proliferation in a subject by administering to the subject a COX2 Ser516 acetylating agent alone or in combination with a cPLA2 agonist provided herein, the administering

decreases ocular fibroblast prostaglandin production;

increases ocular fibroblast 5-hydroxyeicosatetraenoic acid (5-HETE), 15-hydroxyeicosatetraenoic acid (15-HETE) and/or 17-hydroxy-docosahexaenoic acid (17-OHDHA) production;

decreases ocular fibroblast metabolic activity;

decreases ocular fibroblast collagen production;

decreases ocular fibroblast alpha smooth muscle actin (αSMA) expression;

decreases ocular fibroblast marker of proliferation Ki-67 (Ki-67) expression;

decreases ocular fibroblast matrix metalloproteinase (MMP) expression;

increases ocular fibroblast peroxisome proliferator-activated receptor gamma (PPARγ) expression;

decreases ocular fibroblast SMAD family member 2/3 (SMAD2/3) expression; and/or

decreases ocular fibroblast SMAD2/3 phosphorylation.

### BRIEF DESCRIPTION OF THE DRAWINGS

These and other features of the disclosure will become more apparent in the following detailed description and exemplary embodiments in which reference is made to the appended drawings wherein:

FIG. 1A is a graph of the percent of original area of human Tenon's capsule fibroblasts (HTCFs) cast and cultured within type 1 collagen scaffolds and exposed to culture with gentamicin (Gent) (1000 μg/ml), acetylsalicylic acid (ASA) (2000 μg/ml), vehicle control (Dulbecco's Modified Eagle's Medium (DMEM) w/2% fetal bovine serum (FBS)) or combination Gent (1000 μg/ml)+ASA (2000 μg/ml) over a four day period. Scaffolds were detached from walls after 72 hrs to allow cell-mediated collagen contraction to begin. The area of each collagen disk was measured hourly for the first 12 hrs after release, then every 24 hrs until day 4 (mean area remaining at given time point with 95% confidence interval (C), N=4, n=3).

FIG. 1B is an image of HTCFs cast and cultured within type 1 collagen scaffolds after 4 days of contraction with exposure to Gent (1000 μg/ml)+ASA (2000 μg/ml). Scaffolds were detached from walls after 72 hrs for cell-mediated collagen contraction to occur. N=4, n=3.

FIG. 1C is an image of HTCFs cast and cultured within type 1 collagen scaffolds after 4 days of contraction with exposure to ASA (2000 μg/ml). Scaffolds were detached from walls after 72 hrs to allow cell-mediated collagen contraction to begin. N=4, n=3.

FIG. 1D is an image of HTCFs cast and cultured within type 1 collagen scaffolds after 4 days of contraction with exposure to Gent (1000 μg/ml). Scaffolds were detached from walls after 72 hrs to allow cell-mediated collagen contraction to begin. N=4, n=3.

FIG. 1E is an image of HTCFs cast and cultured within type 1 collagen scaffolds after 4 days of contraction with exposure to Vehicle Control (DMEM w/2% FBS). Scaffolds were detached from walls after 72 hrs for cell-mediated collagen contraction to occur. N=4, n=3.

FIG. 2A are images of cell free type 1 collagen scaffolds that were cultured for seven days under self-imposed tension (no detachment) and treated with vehicle control (DMEM w/2% FBS). Scaffolds were fixed in 4% paraformaldehyde, embedded in paraffin, sectioned and stained with picrosirus red to resolve variation in collagen architecture using circularly polarized light microscopy.

FIG. 2B are images of HTCFs cultured within type 1 collagen scaffolds treated with vehicle control (DMEM w/2% FBS) for 7 days under self-imposed tension (no detachment). At experiment conclusion, scaffolds were fixed in 4% paraformaldehyde, embedded in paraffin, sectioned and stained with picrosirus red to resolve variation in collagen architecture using circularly polarized light microscopy.

FIG. 2C are images of HTCFs cultured within type 1 collagen scaffolds treated with ASA (2000 μg/ml) for 7 days under self-imposed tension (no detachment). At experiment conclusion, scaffolds were fixed in 4% paraformaldehyde, embedded in paraffin, sectioned and stained with picrosirus red to resolve variation in collagen architecture using circularly polarized light microscopy.

FIG. 2D are images of HTCFs cultured within type 1 collagen scaffolds treated with Gentamicin (Gent 1000 μg/ml) and ASA (2000 μg/ml) in combination for 7 days under self-imposed tension (no detachment). At experiment conclusion, scaffolds were fixed in 4% paraformaldehyde, embedded in paraffin, sectioned and stained with picrosirus red to resolve variation in collagen architecture using circularly polarized light microscopy.

FIG. 2E is a histogram showing quantitation of the images of cell free and HTCF containing type 1 collagen scaffolds, treated with either: vehicle control (cell free and HTCF), ASA (2000 μg/ml) or combination Gent (1000 μg/ml) and ASA (2000 μg/ml)—examples of which were presented in FIGS. 2A-D. Cell free scaffold staining proportions were significantly different for all colors when comparing the cell free group to the HTCF group (N=4, n=3, p<0.001 for all). There were no statistically significant differences between the HTCF and ASA (2000 μg/ml) groups, although a trend toward less red, yellow and orange staining areas was evident. Replicates and p-values for the comparison between the HTCF group and the Gent (1000 μg/ml)+ASA (2000 μg/ml) group are displayed at the bottom of the figure.

FIG. 3A are images of expression of αSMA (a marker of the myofibroblast phenotype) in HTCFs cast and cultured within type 1 collagen scaffolds and exposed to vehicle control (DMEM w/2% FBS) (Vehicle control (VC); left image) or combination Gent (1000 μg/ml)+ASA (2000 μg/ml) (right image) for 7 days under self-imposed tension (no detachment). At experiment conclusion, scaffolds were fixed in 4% paraformaldehyde, embedded in paraffin, sectioned and stained with primary antibodies against alpha smooth muscle actin (αSMA; a marker of the myofibroblast phenotype), counter stained with florescent secondary antibodies as well as 4′,6-diamidino-2-phenylindole, dihydrochloride (DAPI) for visualization of nuclei. The right panel is a histogram showing relative expression of αSMA, standardized to cell number for each of the treatment groups. (p<0.01, N=3, n=3).

FIG. 3B are images of expression of Ki-67 (a marker of cellular proliferation) in HTCFs cast and cultured within type 1 collagen scaffolds and exposed to vehicle control (DMEM w/2% FBS) (left image) or combination Gent (1000 μg/ml)+ASA (2000 μg/ml) (right image) for 7 days under self-imposed tension (no detachment). At experiment conclusion, scaffolds were fixed in 4% paraformaldehyde, embedded in paraffin, sectioned and stained with primary antibodies against Ki-67 (a marker of cellular proliferation), counter stained with florescent secondary antibodies as well as DAPI for visualization of nuclei. The right panel is a histogram showing relative cellular proliferation for each of the treatment groups. (p<0.001, N=3, n=3).

FIG. 3C are images of cells stained with 4,6-diamidino-2-phenylindole (marker of cell nuclei) in HTCFs cast and cultured within type 1 collagen scaffolds and exposed to vehicle control (DMEM w/2% FBS) (left image) or combination Gent (1000 μg/ml)+ASA (2000 μg/ml) (right image) for 7 days under self-imposed tension (no detachment). At experiment conclusion, scaffolds were fixed in 4% paraformaldehyde, embedded in paraffin, sectioned and stained with 4,6-diamidino-2-phenylindole for 10 minutes. The background autofluorescence of the collagen matrix was imaged at wavelength (500-520 nm) and used to standardize the number of nuclei counted per image using ImageJ. The right panel is a histogram showing relative cellular density (#nuclei/#pixels representing collagen matrix) for each treatment group. (p<0.01).

FIG. 4A are graphs of the mean relative secretion of arachidonic acid (AA; top panel) and eicosapentaenoic acid (EPA; bottom panel) from HTCFs cultured in monolayer and exposed to one of three experimental treatments: 1) vehicle control (DMEM without serum with 1 ng/ml each of IL-1b and TGFb1), 2) ASA (DMEM without serum with 200 μg/ml ASA) or 3) ASA/Mel. (DMEM without serum with 200 μg/ml ASA and 10 μg/ml Melittin) for 24 hours. Supernatant was collected at 6, 12 and 24 hrs after experimental treatment for LC-MS/MS analysis of polyunsaturated fatty acid (PUFA) mediators (N=3; ns=not significant; *p<0.05, **p<0.01, ***p<0.001, ****p<0.0001).

FIG. 4B is a graph of the mean relative secretion of docosahexaenoic acid (DHA) from HTCFs cultured in monolayer and exposed to one of three experimental treatments: 1) vehicle control (DMEM without serum with 1 ng/ml each of IL-1b and TGFb1), 2) ASA (DMEM without serum with 200 μg/ml ASA) or 3) ASA/Mel. (DMEM without serum with 200 μg/ml ASA and 10 μg/ml Melittin) for 24 hours. Supernatant was collected at 6, 12 and 24 hrs after experimental treatment for LC-MS/MS analysis of polyunsaturated fatty acid mediators (N=3; *p<0.05, **p<0.01, ***p<0.001, ****p<0.0001).

FIG. 4C are graphs of the relative secretion of LOX/acetyl-COX2 derived products 5-hydroxyeicosatetraenoic acid (5-HETE; top panel) and 15-hydroxyeicosatetraenoic acid (15-HETE; bottom panel) from HTCFs cultured in monolayer and exposed to one of three experimental treatments: 1) vehicle control (DMEM without serum with 1 ng/ml each of IL-1b and TGFb1), 2) ASA (DMEM without serum with 200 μg/ml ASA) or 3) ASA/Mel (DMEM without serum with 200 μg/ml ASA and 10 μg/ml Melittin) for 24 hours. Supernatant was collected at 6, 12 and 24 hrs after experimental treatment for LC-MS/MS analysis of polyunsaturated fatty acid mediators (N=3; *p<0.05, **p<0.01, ***p<0.001, ****p<0.0001).

FIG. 4D are graphs of the relative secretion of LOX/acetyl-COX2 derived products 17-hydroxy-docosahexaenoic acid (17-OHDHA; top panel) and 18-hydroxyeicosapentaenoic acid (18-HEPE; bottom panel) from HTCFs cultured in monolayer and exposed to one of three experimental treatments: 1) vehicle control (DMEM without serum with 1 ng/ml each of IL-1b and TGFb1), 2) ASA (DMEM without serum with 200 μg/ml ASA) or 3) ASA/Mel (DMEM without serum with 200 μg/ml ASA and 10 μg/ml Melittin) for 24 hours. Supernatant was collected at 6, 12 and 24 hrs after experimental treatment for LC-MS/MS analysis of polyunsaturated fatty acid mediators (N=3; *p<0.05, **p<0.01, ***p<0.001, ****p<0.0001).

FIG. 4E are graphs of the relative secretion of COX2 derived products 6-keto-prostaglandin F1α (kPGF1a; top panel) and prostaglandin E2 (PGE2; bottom panel) from HTCFs cultured in monolayer and exposed to one of three experimental treatments: 1) vehicle control (DMEM without serum with 1 ng/ml each of IL-1b and TGFb1), 2) ASA (DMEM without serum with 200 μg/ml ASA) or 3) ASA/Mel (DMEM without serum with 200 μg/ml ASA and 10 μg/ml Melittin) for 24 hours. Supernatant was collected at 6, 12 and 24 hrs after experimental treatment for LC-MS/MS analysis of polyunsaturated fatty acid mediators (N=3; *p<0.05, **p<0.01, ***p<0.001, ****p<0.0001).

FIG. 5A are graphs of the mean relative secretion of arachidonic acid (AA; top panel) and eicosapentaenoic acid (EPA; bottom panel) from HTCFs cultured in monolayer and exposed to one of four experimental treatments: 1) vehicle control (DMEM without serum), 2) inflammatory cytokines (Inf. Cyto.), an inflammation induced positive control (DMEM without serum with 1 ng/ml each of IL-1b, IFNγ, TNFa and TGFb1), 3) Inf. Cyto./Gent250 (DMEM without serum with 250 μg/ml gentamicin and 1 ng/ml each of IL-1b, IFNγ, TNFa and TGFb1), 4) Inf. Cyto./Gent500 (DMEM without serum with 500 μg/ml gentamicin and 1 ng/ml each of IL-1b, IFNγ, TNFa and TGFb1) for 24 hours. Supernatant was collected at 6, 12 and 24 hrs after experimental treatment for LC-MS/MS analysis of polyunsaturated fatty acid mediators (N=4; *p<0.05, **p<0.01, ***p<0.001, ****p<0.0001).

FIG. 5B is a graph of the mean relative secretion of docosahexaenoic acid (DHA) from HTCFs cultured in monolayer and exposed to one of four experimental treatments: 1) vehicle control (DMEM without serum), 2) inflammatory cytokines (Inf. Cyto.), an inflammation induced positive control (DMEM without serum with 1 ng/ml each of IL-1b, IFNγ, TNFa and TGFb1), 3) Inf. Cyto./Gent250 (DMEM without serum with 250 μg/ml gentamicin and 1 ng/ml each of IL-1b, IFNγ, TNFa and TGFb1), 4) Inf. Cyto./Gent500 (DMEM without serum with 500 μg/ml gentamicin and 1 ng/ml each of IL-1b, IFNγ, TNFa and TGFb1) for 24 hours. Supernatant was collected at 6, 12 and 24 hrs after experimental treatment for LC-MS/MS analysis of polyunsaturated fatty acid mediators (N=4; *p<0.05, **p<0.01, ***p<0.001,****p<0.0001).

FIG. 5C are graphs of the relative secretion of COX2 derived products prostaglandin E2 (PGE2; top panel) and 6-keto-prostaglandin F1α (kPGF1a; bottom panel) from HTCFs cultured in monolayer and exposed to one of five experimental treatments: 1) vehicle control (DMEM without serum), 2) CytoM1+TGFB1, an inflammation induced positive control (DMEM without serum with 1 ng/ml each of IL-1b, IFNγ, TNFa and TGFb1), 3) Gent 250 (DMEM without serum with 250 μg/ml gentamicin and 1 ng/ml each of IL-1b, IFNγ, TNFa and TGFb1), 4) ASA 500 (DMEM without serum with 500 μg/ml ASA and 1 ng/ml each of IL-1b, IFNγ, TNFa and TGFb1), 5) ASA 500+Gent 250 (DMEM without serum with 500 μg/ml ASA, 250 μg/ml gentamicin and 1 ng/ml each of IL-1b, IFNγ, TNFa and TGFb1) for 48 hours. Supernatant was collected at 6, 12, 24 and 48 hrs after experimental treatment for LC-MS/MS analysis of polyunsaturated fatty acid mediators (N=4; ns=not significant; *p<0.05, **p<0.01.***p<0.001.****p<0.0001).

FIG. 5D are graphs of the relative secretion of COX2 derived products prostaglandin E2 (PGE2; top panel) and 6-keto-prostaglandin F1α (kPGF1a; bottom panel) from HTCFs cultured in monolayer and exposed to one of five experimental treatments: 1) vehicle control (DMEM without serum), 2) CytoM1+TGFB1, an inflammation induced positive control (DMEM without serum with 1 ng/ml each of IL-1b, IFNγ, TNFa and TGFb1), 3) Gent 500 (DMEM without serum with 500 μg/ml gentamicin and 1 ng/ml each of IL-1b, IFNγ, TNFa and TGFb1), 4) ASA 1000 (DMEM without serum with 1000 μg/ml ASA and 1 ng/ml each of IL-1b, IFNγ, TNFa and TGFb1), 5) ASA 1000+Gent 500 (DMEM without serum with 1000 μg/ml ASA, 500 μg/ml gentamicin and 1 ng/ml each of IL-1b, IFNγ, TNFa and TGFb1) for 48 hours. Supernatant was collected at 6, 12, 24 and 48 hrs after experimental treatment for LC-MS/MS analysis of polyunsaturated fatty acid mediators (N=4; ns=not significant; *p<0.05, **p<0.01, ***p<0.001, ****p<0.0001).

FIG. 5E are graphs of the relative secretion of COX2 derived products prostaglandin E2 (PGE2; top panel) and 6-keto-prostaglandin F1α (kPGF1a; bottom panel) from HTCFs cultured in monolayer and exposed to one of five experimental treatments: 1) vehicle control (DMEM without serum), 2) CytoM1+TGFB1, an inflammation induced positive control (DMEM without serum with 1 ng/ml each of IL-1b, IFNγ, TNFa and TGFb1), 3) Gent 250 (DMEM without serum with 250 μg/ml gentamicin and 1 ng/ml each of IL-1b, IFNγ, TNFa and TGFb1), 4) APHS 12 (DMEM without serum with 12 μg/ml o-(acetoxyphenyl)hept-2-ynyl sulfide (APHS) and 1 ng/ml each of IL-1b, IFNγ, TNFa and TGFb1), 5) APHS 12+Gent 250 (DMEM without serum with 12 μg/ml o-(acetoxyphenyl)hept-2-ynyl sulfide (APHS), 250 μg/ml gentamicin and 1 ng/ml each of IL-1b, IFNγ, TNFa and TGFb1) for 48 hours. Supernatant was collected at 6, 12, 24 and 48 hrs after experimental treatment for LC-MS/MS analysis of polyunsaturated fatty acid mediators (N=3; ns=not significant; *p<0.05, **p<0.01, ***p<0.001, ****p<0.0001).

FIG. 6A shows are graphs of the relative secretion of LOX/acetyl-COX2 derived products 5-hydroxyeicosatetraenoic acid (5-HETE; top panel) and 15-hydroxyeicosatetraenoic acid (15-HETE; bottom panel) from HTCFs cultured in monolayer and exposed to one of five experimental treatments: 1) vehicle control (DMEM without serum), 2) CytoM1+TGFB1, an inflammation induced positive control (DMEM without serum with 1 ng/ml each of IL-1b, IFNγ, TNFa and TGFb1), 3) Gent 250 (DMEM without serum with 250 μg/ml gentamicin and 1 ng/ml each of IL-1b, IFNγ, TNFa and TGFb1), 4) ASA 500 (DMEM without serum with 500 μg/ml ASA and 1 ng/ml each of IL-1b, IFNγ, TNFa and TGFb1), 5) ASA 500+Gent 250 (DMEM without serum with 500 μg/ml ASA, 250 μg/ml gentamicin and 1 ng/ml each of IL-1b, IFNγ, TNFa and TGFb1) for 48 hours. Supernatant was collected at 6, 12, 24 and 48 hrs after experimental treatment for LC-MS/MS analysis of polyunsaturated fatty acid mediators (N=3; *p<0.05, **p<0.01, ***p<0.001, ****p<0.0001).

FIG. 6B are graphs of the relative secretion of LOX/acetyl-COX2 derived products 5-hydroxyeicosatetraenoic acid (5-HETE; top panel) and 15-hydroxyeicosatetraenoic acid (15-HETE; bottom panel) from HTCFs cultured in monolayer and exposed to one of five experimental treatments: 1) vehicle control (DMEM without serum), 2) CytoM1+TGFB1, an inflammation induced positive control (DMEM without serum with 1 ng/ml each of IL-1b, IFNγ, TNFa and TGFb1), 3) Gent 500 (DMEM without serum with 500 μg/ml gentamicin and 1 ng/ml each of IL-1b, IFNγ, TNFa and TGFb1), 4) ASA 1000 (DMEM without serum with 1000 μg/ml ASA and 1 ng/ml each of IL-1b, IFNγ, TNFa and TGFb1), 5) ASA 1000+Gent 500 (DMEM without serum with 1000 μg/ml ASA, 500 μg/ml gentamicin and 1 ng/ml each of IL-1b, IFNγ, TNFa and TGFb1) for 48 hours. Supernatant was collected at 6, 12, 24 and 48 hrs after experimental treatment for LC-MS/MS analysis of polyunsaturated fatty acid mediators (N=3; *p<0.05, **p<0.01, ***p<0.001, ****p<0.0001).

FIG. 6C are graphs of the relative secretion of LOX/acetyl-COX2 derived products 5-hydroxyeicosatetraenoic acid (5-HETE; top panel) and 15-hydroxyeicosatetraenoic acid (15-HETE; bottom panel) from HTCFs cultured in monolayer and exposed to one of five experimental treatments: 1) vehicle control (DMEM without serum), 2) CytoM1+TGFB1, an inflammation induced positive control (DMEM without serum with 1 ng/ml each of IL-1b, IFNγ, TNFa and TGFb1), 3) Gent 250 (DMEM without serum with 250 μg/ml gentamicin and 1 ng/ml each of IL-1b, IFNγ, TNFa and TGFb1), 4) APHS 12 (DMEM without serum with 12 μg/ml o-(acetoxyphenyl)hept-2-ynyl sulfide (APHS) and 1 ng/ml each of IL-1b, IFNγ, TNFa and TGFb1), 5) APHS 12+Gent 250 (DMEM without serum with 12 μg/ml o-(acetoxyphenyl)hept-2-ynyl sulfide (APHS), 250 μg/ml gentamicin and 1 ng/ml each of IL-1b, IFNγ, TNFa and TGFb1) for 48 hours. Supernatant was collected at 6, 12, 24 and 48 hrs after experimental treatment for LC-MS/MS analysis of polyunsaturated fatty acid mediators (N=3; *p<0.05, **p<0.01, ***p<0.001, ****p<0.0001).

FIG. 7A is a graph of the mean relative secretion of COX derived products (PGE2 and kPGF1a) subtracted from the mean relative secretion of LOX/acetyl-COX2 derived products (5-HETE and 15-HETE) from HTCFs cultured in monolayer and exposed to one of three experimental treatments: 1) Inf. Cytokines (DMEM without serum with 1 ng/ml each of IL-1b, IFNγ, TNFa and TGFb1), 2) Inf. Cyto./ASA200 (DMEM without serum with 200 μg/ml ASA and 1 ng/ml each of IL-1b, IFNγ, TNFa and TGFb1) or 3) Inf. Cyto./ASA200/Mel.10 (DMEM without serum with 200 μg/ml ASA, 10 μg/ml Melittin and 1 ng/ml each of IL-1b, IFNγ, TNFa and TGFb1) for 24 hours. Supernatant was collected at 6, 12 and 24 hrs after experimental treatment for LC-MS/MS analysis of polyunsaturated fatty acid mediators (N=3; *p<0.05, **p<0.01, ***p<0.001, ****p<0.0001).

FIG. 7B is a graph of the mean relative secretion of COX derived products (PGE2 and kPGF1a) subtracted from the mean relative secretion of LOX/acetyl-COX2 derived products (5-HETE and 15-HETE) from HTCFs cultured in monolayer and exposed to one of five experimental treatments: 1) vehicle control (DMEM without serum), 2) Inf. Cytokines, an inflammation induced positive control (DMEM without serum with 1 ng/ml each of IL-1b, IFNγ, TNFa and TGFb1), 3) Gent 250 (DMEM without serum with 250 μg/ml gentamicin and 1 ng/ml each of IL-1b, IFNγ, TNFa and TGFb1), 4) ASA 500 (DMEM without serum with 500 μg/ml ASA and 1 ng/ml each of IL-1b, IFNγ, TNFa and TGFb1), 5) ASA 500+Gent 250 (DMEM without serum with 500 μg/ml ASA, 250 μg/ml gentamicin and 1 ng/ml each of IL-1b, IFNγ, TNFa and TGFb1) for 48 hours. Supernatant was collected at 6, 12, 24 and 48 hrs after experimental treatment for LC-MS/MS analysis of polyunsaturated fatty acid mediators (N=3; *p<0.05, **p<0.01, ***p<0.001, ****p<0.0001).

FIG. 7C is a graph of the mean relative secretion of COX derived products (PGE2 and kPGF1a) subtracted from the mean relative secretion of LOX/acetyl-COX2 derived products (5-HETE and 15-HETE) from HTCFs cultured in monolayer and exposed to one of five experimental treatments: 1) vehicle control (DMEM without serum), 2) Inf. Cytokines, an inflammation induced positive control (DMEM without serum with 1 ng/ml each of IL-1b, IFNγ, TNFa and TGFb1), 3) Gent 500 (DMEM without serum with 500 μg/ml gentamicin and 1 ng/ml each of IL-1b, IFNγ, TNFa and TGFb1), 4) ASA 1000 (DMEM without serum with 1000 μg/ml ASA and 1 ng/ml each of IL-1b, IFNγ, TNFa and TGFb1), 5) ASA 1000+Gent 500 (DMEM without serum with 1000 μg/ml ASA, 500 μg/ml gentamicin and 1 ng/ml each of IL-1b, IFNγ, TNFa and TGFb1) for 48 hours. Supernatant was collected at 6, 12, 24 and 48 hrs after experimental treatment for LC-MS/MS analysis of polyunsaturated fatty acid mediators (N=3; *p<0.05, **p<0.01, ***p<0.001, ****p<0.0001.

FIG. 7D is a graph of the mean relative secretion of COX derived products (PGE2 and kPGF1a) subtracted from the mean relative secretion of LOX/acetyl-COX2 derived products (5-HETE and 15-HETE from HTCFs cultured in monolayer and exposed to one of five experimental treatments: 1) vehicle control (DMEM without serum), 2) Inf. Cytokines, an inflammation induced positive control (DMEM without serum with 1 ng/ml each of IL-1b, IFNγ, TNFa and TGFb1), 3) Gent 250 (DMEM without serum with 250 μg/ml gentamicin and 1 ng/ml each of IL-1b, IFNγ, TNFa and TGFb1), 4) APHS 12 (DMEM without serum with 12 μg/ml o-(acetoxyphenyl)hept-2-ynyl sulfide (APHS) and 1 ng/ml each of IL-1b, IFNγ, TNFa and TGFb1), 5) APHS 12+Gent 250 (DMEM without serum with 12 μg/ml o-(acetoxyphenyl)hept-2-ynyl sulfide (APHS), 250 μg/ml gentamicin and 1 ng/ml each of IL-1b, IFNγ, TNFa and TGFb1) for 48 hours. Supernatant was collected at 6, 12, 24 and 48 hrs after experimental treatment for LC-MS/MS analysis of polyunsaturated fatty acid mediators (N=3; *p<0.05, **p<0.01, ***p<0.001, ****p<0.0001).

FIG. 8A is a graph of the percent of original area of type 1 collagen scaffolds containing human Tenon's capsule fibroblasts (HTCFs) that, after a 72 hr incubation under normal culture conditions, were subsequently detached from the sides of the culture wells and allowed to freely contract while exposed to one of six experimental treatments: 1) vehicle control (DMEM with 2% FBS), 2) DMEM w/2% FBS and 100 μg/ml gentamicin, 3) DMEM w/2% FBS and 250 μg/ml gentamicin, 4) DMEM w/2% FBS and 500 μg/ml gentamicin, 5) DMEM w/2% FBS and 750 μg/ml gentamicin and 6) DMEM w/2% FBS and 1000 μg/ml gentamicin for a period of four days. Measurements of collagen scaffold surface area were taken on days 1, 2, 3 and 4 to assess changes from baseline. Results of a two-way repeated measures analysis of variance statistical test are also displayed; there were no significant treatment effects found.

FIG. 8B is a graph of the percent of original area of type 1 collagen scaffolds containing human Tenon's capsule fibroblasts (HTCFs) that, after a 72 hr incubation under normal culture conditions, were subsequently detached from the sides of the culture wells and allowed to freely contract while exposed to one of four experimental treatments: 1) vehicle control (DMEM with 2% FBS), 2) DMEM w/2% FBS and 500 μg/ml ASA, 3) DMEM w/2% FBS and 1000 μg/ml ASA and 4) DMEM w/2% FBS and 1500 μg/ml ASA for a period of four days. Measurements of collagen scaffold surface area were taken on days 1, 2, 3 and 4 to assess changes from baseline. Results of a two-way repeated measures analysis of variance statistical test are also displayed; ****p<0.0001.

FIG. 8C is a graph of the percent of original area of type 1 collagen scaffolds containing human Tenon's capsule fibroblasts (HTCFs) that, after a 72 hr incubation under normal culture conditions, were subsequently detached from the sides of the culture wells and allowed to freely contract while exposed to one of seven experimental treatments: 1) vehicle control (DMEM with 2% FBS), 2) DMEM w/2% FBS and 500 μg/ml ASA, 3) DMEM w/2% FBS, 500 μg/ml ASA and 100 μg/ml gentamicin, 4) DMEM w/2% FBS, 500 μg/ml ASA and 250 μg/ml gentamicin, 5) DMEM w/2% FBS, 500 μg/ml ASA and 500 μg/ml gentamicin, 6) DMEM w/2% FBS, 500 μg/ml ASA and 750 μg/ml gentamicin, and 7) DMEM w/2% FBS, 500 μg/ml ASA and 1000 μg/ml gentamicin for a period of four days. Measurements of collagen scaffold surface area were taken on days 1, 2, 3 and 4 to assess changes from baseline. Results of a two-way repeated measures analysis of variance statistical test are also displayed; there were no significant treatment effects found.

FIG. 8D is a graph of the percent of original area of type 1 collagen scaffolds containing human Tenon's capsule fibroblasts (HTCFs) that, after a 72 hr incubation under normal culture conditions, were subsequently detached from the sides of the culture wells and allowed to freely contract while exposed to one of seven experimental treatments: 1) vehicle control (DMEM with 2% FBS), 2) DMEM w/2% FBS and 1000 μg/ml ASA, 3) DMEM w/2% FBS, 1000 μg/ml ASA and 100 μg/ml gentamicin, 4) DMEM w/2% FBS, 1000 μg/ml ASA and 250 μg/ml gentamicin, 5) DMEM w/2% FBS, 1000 μg/ml ASA and 500 μg/ml gentamicin, 6) DMEM w/2% FBS, 1000 μg/ml ASA and 750 μg/ml gentamicin, and 7) DMEM w/2% FBS, 1000 μg/ml ASA and 1000 μg/ml gentamicin for a period of four days. Measurements of collagen scaffold surface area were taken on days 1, 2, 3 and 4 to assess changes from baseline. Results of a two-way repeated measures analysis of variance statistical test are also displayed; ****p<0.0001.

FIG. 8E is a graph of the percent of original area of type 1 collagen scaffolds containing human Tenon's capsule fibroblasts (HTCFs) that, after a 72 hr incubation under normal culture conditions, were subsequently detached from the sides of the culture wells and allowed to freely contract while exposed to one of seven experimental treatments: 1) vehicle control (DMEM with 2% FBS), 2) DMEM w/2% FBS and 1500 μg/ml ASA, 3) DMEM w/2% FBS, 1500 μg/ml ASA and 100 μg/ml gentamicin, 4) DMEM w/2% FBS, 1500 μg/ml ASA and 250 μg/ml gentamicin, 5) DMEM w/2% FBS, 1500 μg/ml ASA and 500 μg/ml gentamicin, 6) DMEM w/2% FBS, 1500 μg/ml ASA and 750 μg/ml gentamicin, and 7) DMEM w/2% FBS, 1500 μg/ml ASA and 1000 μg/ml gentamicin for a period of four days. Measurements of collagen scaffold surface area were taken on days 1, 2, 3 and 4 to assess changes from baseline. Results of a two-way repeated measures analysis of variance statistical test are also displayed; *p<0.05, ****p<0.0001.

FIG. 9 is a graph of the percent of original area of type 1 collagen scaffolds containing human Tenon's capsule fibroblasts (HTCFs) that, after a 72 hr incubation under normal culture conditions, were subsequently detached from the sides of the culture wells and allowed to freely contract while exposed to one of eight experimental treatments: 1) vehicle control (DMEM w/2% FBS), 2) pH control (DMEM w/2% FBS and hydrochloric acid added equimolar to 1500 μg/ml ASA so as to provide equivalent H⁺ to the buffered culture media), 3) 1000ASA (DMEM w/2% FBS and 1000 μg/ml ASA), 4) 1000ASA+333G (DMEM w/2% FBS, 1000 μg/ml ASA and 333 μg/ml gentamicin), 5) 1000ASA+500G (DMEM w/2% FBS, 1000 μg/ml ASA and 500 μg/ml gentamicin), 6) 1500ASA (DMEM w/2% FBS and 1500 μg/ml ASA), 7) 1500ASA+500G (DMEM w/2% FBS, 1500 μg/ml ASA and 500 μg/ml gentamicin), 8) 1500ASA+750G (DMEM w/2% FBS, 1500 μg/ml ASA and 750 μg/ml gentamicin) for a period of four days. During contraction, at the 48 hr timepoint, 2 ng/ml of TGFB1 was added to the culture media of all treatment groups. Measurements of collagen scaffold surface area were taken on days 1, 2, 3 and 4 to assess changes from baseline. Results of a two-way repeated measures analysis of variance statistical test are indicated. Significant differences between 1500 μg/ml ASA+750 μg/ml gentamicin and 1500 μg/ml ASA are indicated (N=3; *p<0.05, **p<0.01, ***p<0.001, ****p<0.0001).

FIG. 10A are fluorescent micrographs of human Tenon's capsule fibroblasts (HTCFs) cultured for seven days within a type 1 collagen scaffold, during the last four days of the experiment the cultures were treated with one of four experimental treatments: 1) vehicle control (DMEM with 2% FBS, 2) DMEM w/2% FBS and 1000 μg/ml ASA, 3) DMEM w/2% FBS, 1000 μg/ml ASA and 333 μg/ml gentamicin, 4) DMEM w/2% FBS, 1000 μg/ml ASA and 500 μg/ml gentamicin. At the conclusion of the experiment, the cells were stained for 5 min with fluorescein diacetate and propidium iodide prior to fluorescent microscopy to visualize living/viable vs. damaged cells.

FIG. 10B are fluorescent micrographs of human Tenon's capsule fibroblasts (HTCFs) cultured for seven days within a type 1 collagen scaffold, during the last four days of the experiment the cultures were treated with one of four experimental treatments: 1) vehicle control (DMEM with 2% FBS), 2) DMEM w/2% FBS and 1500 μg/ml ASA, 3) DMEM w/2% FBS, 1500 μg/ml ASA and 500 μg/ml gentamicin, 4) DMEM w/2% FBS, 1500 μg/ml ASA and 1000 μg/ml gentamicin. At the conclusion of the experiment, the cells were stained for 5 min with fluorescein diacetate and propidium iodide prior to fluorescent microscopy to visualize living/viable vs. damaged cells.

FIG. 10C are fluorescent micrographs of human Tenon's capsule fibroblasts (HTCFs) cultured for seven days within a type 1 collagen scaffold, during the last four days of the experiment the cultures were treated with one of two experimental treatments: 1) vehicle control (DMEM with 2% FBS) and 2) DMEM w/2% FBS as well as hydrochloric acid added such that the disassociated H⁺ ions were equimolar to 1500 μg/ml of ASA. At the conclusion of the experiment, the cells were stained for 5 min with fluorescein diacetate and propidium iodide prior to fluorescent microscopy to visualize living/viable vs. damaged cells.

FIG. 10D is a graph of the ratio of viable cells to total cells as measured by fluorescent microscopy and fluorescein diacetate/propidium iodide staining for each experimental treatment group. Human Tenon's capsule fibroblasts (HTCFs) were cultured for seven days within type 1 collagen scaffolds, during the last four days of the experiment the cultures were treated with one of eight experimental treatments: 1) vehicle control (DMEM with 2% FBS), 2) pH control (DMEM w/2% FBS and hydrochloric acid added equimolar to 1500 μg/ml ASA so as to provide equivalent H⁺ to the buffered culture media), 3) DMEM w/2% FBS and 1000 μg/ml ASA, 4) DMEM w/2% FBS, 1000 μg/ml ASA and 333 μg/ml gentamicin, 5) DMEM w/2% FBS, 1000 μg/ml ASA and 500 μg/ml gentamicin, 6) DMEM w/2% FBS and 1500 μg/ml ASA, 7) DMEM w/2% FBS, 1500 μg/ml ASA and 500 μg/ml gentamicin, 8) DMEM w/2% FBS, 1500 μg/ml ASA and 750 μg/ml gentamicin for a period of four days.

FIG. 11A is a graph of the percent of original area of type 1 collagen scaffolds containing human Tenon's capsule fibroblasts (HTCFs) that, after a 72 hr incubation under normal culture conditions, were subsequently detached from the sides of the culture wells and allowed to freely contract while exposed to one of eight experimental treatments: 1) vehicle control (DMEM with 2% FBS), 2) aqueous humor growth factors (AHGFs: DMEM w/2% FBS and 2 ng/ml each of TGFB1, TGFB2, VEGF, CCN2), 3) AHGFs/Gent750 (DMEM w/2% FBS, 2 ng/ml each of TGFB1, TGFB2, VEGF, CCN2 and 750 μg/ml gentamicin), 4) AHGFs/ASA1500 (DMEM w/2% FBS, 2 ng/ml each of TGFB1, TGFB2, VEGF, CCN2 and 1500 μg/ml ASA), 5) AHGFs/ASA1500/Gent750 (DMEM w/2% FBS, 2 ng/ml each of TGFB1, TGFB2, VEGF, CCN2, 1500 μg/ml ASA and 750 μg/ml gentamicin), and 6) MMC4 (before detachment of these collagen scaffolds from culture wells, they were exposed to 0.2 mg/ml mitomycin C in PBS for 4 minutes prior to being subsequently washed twice with fresh PBS. DMEM w/2% FBS was added for the remainder of the experiment). Measurements of collagen scaffold surface area were taken on days 1, 2, 3 and 4 to assess changes from baseline. Results of a two-way repeated measures analysis of variance statistical test are indicated (N=5, n=3; *p<0.05, **p<0.01, ***p<0.001, ****p<0.0001).

FIG. 11B shows representative images of type 1 collagen scaffolds containing HTCFs from the experiment shown in FIG. 11A. The images were captured after 96 hrs of culture.

FIG. 12A are fluorescent micrographs of human Tenon's capsule fibroblasts (HTCFs) cultured for seven days within a type 1 collagen scaffold, during the last four days of the experiment the cultures were treated with one of four experimental treatments: 1) AHGFs control (DMEM with 2% FBS and 2 ng/ml each of TGFB1, TGFB2, VEGF, CCN2), 2) AHGFs/ASA1500 (DMEM w/2% FBS, 1500 μg/ml ASA and 2 ng/ml each of TGFB1, TGFB2, VEGF, CCN2), 3) AHGFs/ASA1500/Gent750 (DMEM w/2% FBS, 1500 μg/ml ASA, 750 μg/ml gentamicin and 2 ng/ml each of TGFB1, TGFB2, VEGF, CCN2), 4) AHGFs/MMC4 (before detachment of these collagen scaffolds from culture wells, they were exposed to 0.2 mg/ml mitomycin C in PBS for 4 minutes prior to being subsequently washed twice with fresh PBS. Then DMEM w/2% FBS and 2 ng/ml each of TGFB1, TGFB2, VEGF, CCN2 was added for the remainder of the experiment). At the conclusion of the experiment, the cells were stained for 5 min with fluorescein diacetate and propidium iodide prior to fluorescent microscopy to visualize living/viable vs. damaged cells.

FIG. 12B is a graph of the ratio of viable cells to total cells as measured by fluorescent microscopy and fluorescein diacetate/propidium iodide staining for each experimental treatment group. Human Tenon's capsule fibroblasts (HTCFs) were cultured for seven days within type 1 collagen scaffolds, during the last four days of the experiment the cultures were treated with one of seven experimental treatments: 1) vehicle control (DMEM with 2% FBS), 2) AHGFs control (DMEM with 2% FBS and 2 ng/ml each of TGFB1, TGFB2, VEGF, CCN2), 3) AHGFs/ASA1500 (DMEM w/2% FBS, 1500 μg/ml ASA and 2 ng/ml each of TGFB1, TGFB2, VEGF, CCN2), 4) AHGFs/ASA1500/Gent750 (DMEM w/2% FBS, 1500 μg/ml ASA, 750 μg/ml gentamicin and 2 ng/ml each of TGFB1, TGFB2, VEGF, CCN2), and 5) AHGFs/MMC 1 min (before detachment of these collagen scaffolds from culture wells, they were exposed to 0.2 mg/ml mitomycin C in PBS for 1 minute prior to being subsequently washed twice with fresh PBS. (DMEM with 2% FBS and 2 ng/ml each of TGFB1, TGFB2, VEGF and CCN2 was added for the remainder of the experiment), 6) MMC2 (same as MMC1 except with a 2-minute MMC exposure time), and 7) MMC4 (same as MMC1 and MMC2 except with a 4-minute MMC exposure time). Results of a one-way repeated measures analysis of variance statistical test are indicated (****p<0.001).

FIG. 13A is a graph of the percent of original area of type 1 collagen scaffolds containing human Tenon's capsule fibroblasts (HTCFs) that, after a 72 hr incubation under normal culture conditions, were subsequently detached from the sides of the culture wells and allowed to freely contract while exposed to one of five experimental treatments: 1) aqueous humor growth factors (AHGFs; DMEM w/2% FBS and 2 ng/ml each of TGFB1, TGFB2, VEGF, CCN2), 2) AHGFs/Gent750 (DMEM w/2% FBS, 2 ng/ml each of TGFB1, TGFB2, VEGF, CCN2 and 750 μg/ml gentamicin), 3) AHGFs/APHS 12 (DMEM w/2% FBS, 2 ng/ml each of TGFB1, TGFB2, VEGF, CCN2 and 12 μg/ml o-(acetoxyphenyl)hept-2-ynyl sulfide (APHS)), 4) AHGFs/APHS12/Gent750 (DMEM w/2% FBS, 2 ng/ml each of TGFB1, TGFB2, VEGF, CCN2, 12 μg/ml o-(acetoxyphenyl)hept-2-ynyl sulfide (APHS) and 750 μg/ml gentamicin), and 5) MMC4/AHGFs (before detachment of these collagen scaffolds from culture wells, they were exposed to 0.2 mg/ml mitomycin C in PBS for 4 minutes prior to being subsequently washed twice with fresh PBS. DMEM w/2% FBS and 2 ng/ml each of TGFB1, TGFB2, VEGF, CCN2 was added for the remainder of the experiment). Measurements of collagen scaffold surface area were taken on days 1, 2, 3 and 4 to assess changes from baseline. Results of a two-way repeated measures analysis of variance statistical test are indicated (*p<0.05, **p<0.01, ***p<0.001, ****p<0.0001).

FIG. 13B is a graph of the percent of original area of type 1 collagen scaffolds containing human Tenon's capsule fibroblasts (HTCFs) that, after a 72 hr incubation under normal culture conditions, were subsequently detached from the sides of the culture wells and allowed to freely contract while exposed to one of five experimental treatments: 1) aqueous humor growth factors (AHGFs: DMEM w/2% FBS and 2 ng/ml each of TGFB1, TGFB2, VEGF, CCN2), 2) AHGFs/Gent750 (DMEM w/2% FBS, 2 ng/ml each of TGFB1, TGFB2, VEGF, CCN2 and 750 μg/ml gentamicin), 3) AHGFs/APHS 24 (DMEM w/2% FBS, 2 ng/ml each of TGFB1, TGFB2, VEGF, CCN2 and 24 μg/ml o-(acetoxyphenyl)hept-2-ynyl sulfide (APHS)), 4) AHGFs/APHS24/Gent750 (DMEM w/2% FBS, 2 ng/ml each of TGFB1, TGFB2, VEGF, CCN2, 24 μg/ml o-(acetoxyphenyl)hept-2-ynyl sulfide (APHS) and 750 μg/ml gentamicin), and 5) MMC4/AHGFs (before detachment of these collagen scaffolds from culture wells, they were exposed to 0.2 mg/ml mitomycin C in PBS for 4 minutes prior to being subsequently washed twice with fresh PBS. DMEM w/2% FBS and 2 ng/ml each of TGFB1, TGFB2, VEGF, CCN2 was added for the remainder of the experiment). Measurements of collagen scaffold surface area were taken on days 1, 2, 3 and 4 to assess changes from baseline. Results of a two-way repeated measures analysis of variance statistical test are indicated (*p<0.05, **p<0.01, ***p<0.001, ****p<0.0001).

FIG. 13C are images of HTCFs cast and cultured within type 1 collagen scaffolds after 4 days of contraction with exposure to one of eight experimental treatment conditions: 1) aqueous humor growth factors (AHGFs; DMEM w/2% FBS and 2 ng/ml each of TGFB1, TGFB2, VEGF, CCN2), 2) AHGFs/Gent750 (DMEM w/2% FBS, 2 ng/ml each of TGFB1, TGFB2, VEGF, CCN2 and 750 μg/ml gentamicin), 3) AHGFs/APHS 12 (DMEM w/2% FBS, 2 ng/ml each of TGFB1, TGFB2, VEGF, CCN2 and 12 μg/ml o-(acetoxyphenyl)hept-2-ynyl sulfide (APHS)), 4) AHGFs/APHS12/Gent750 (DMEM w/2% FBS, 2 ng/ml each of TGFB1, TGFB2, VEGF, CCN2, 12 μg/ml o-(acetoxyphenyl)hept-2-ynyl sulfide (APHS) and 750 μg/ml gentamicin), 5) AHGFs/APHS 24 (DMEM w/2% FBS, 2 ng/ml each of TGFB1, TGFB2, VEGF, CCN2 and 24 μg/ml o-(acetoxyphenyl)hept-2-ynyl sulfide (APHS)), 6) AHGFs/APHS24/Gent750 (DMEM w/2% FBS, 2 ng/ml each of TGFB1, TGFB2, VEGF, CCN2, 24 μg/ml o-(acetoxyphenyl)hept-2-ynyl sulfide (APHS) and 750 μg/ml gentamicin), 7) MMC2/AHGFs (before detachment of these collagen scaffolds from culture wells, they were exposed to 0.2 mg/ml mitomycin C in PBS for 2 minutes prior to being subsequently washed twice with fresh PBS. DMEM w/2% FBS and 2 ng/ml each of TGFB1, TGFB2, VEGF, CCN2 was added for the remainder of the experiment) and 8) MMC4/AHGFs (same as MMC2/AHGFs except with a 4-minute MMC exposure time). Scaffolds were detached from walls after 72 hrs to allow cell-mediated collagen contraction to begin.

FIG. 14A are fluorescent micrographs of human Tenon's capsule fibroblasts (HTCFs) cultured for seven days within a type 1 collagen scaffold, during the last four days of the experiment the cultures were treated with one of three experimental treatments: 1) aqueous humor growth factors (AHGFs; DMEM w/2% FBS and 2 ng/ml each of TGFB1, TGFB2, VEGF, CCN2 and 2) AHGFs/APHS24 (DMEM w/2% FBS, 2 ng/ml each of TGFB1, TGFB2, VEGF, CCN2 and 24 μg/ml o-(acetoxyphenyl)hept-2-ynyl sulfide (APHS)), and 3) AHGFs/APHS24/Gent750 (DMEM w/2% FBS, 2 ng/ml each of TGFB1, TGFB2, VEGF, CCN2, 24 μg/ml o-(acetoxyphenyl)hept-2-ynyl sulfide (APHS) and 750 μg/ml gentamicin). At the conclusion of the experiment, the cells were stained for 5 min with fluorescein diacetate and propidium iodide prior to fluorescent microscopy to visualize living/viable vs. damaged cells.

FIG. 14B is a graph of the ratio of viable cells to total cells as measured by fluorescent microscopy and fluorescein diacetate/propidium iodide staining for each experimental treatment group. Human Tenon's capsule fibroblasts (HTCFs) were cultured for seven days within type 1 collagen scaffolds, during the last four days of the experiment the cultures were treated with one of three experimental treatments: 1) aqueous humor growth factors (AHGFs; DMEM w/2% FBS and 2 ng/ml each of TGFB1, TGFB2, VEGF, CCN2), and 2) AHGFs/APHS24 (DMEM w/2% FBS, 2 ng/ml each of TGFB1, TGFB2, VEGF, CCN2 and 24 μg/ml o-(acetoxyphenyl)hept-2-ynyl sulfide (APHS)), and 3) AHGFs/APHS24/Gent750 (DMEM w/2% FBS, 2 ng/ml each of TGFB1, TGFB2, VEGF, CCN2, 24 μg/ml o-(acetoxyphenyl)hept-2-ynyl sulfide (APHS) and 750 μg/ml gentamicin).

FIG. 15A is a graph of the relative metabolic activity of HTCFs (grey bars) and TGFb1-induced HTCFs (black bars) when exposed to different concentrations of ASA. HTCFs were cultured in monolayer and treated with one of the following experimental treatments for 48 hrs: 1) vehicle control (DMEM w/0% FBS), 2) TGFb1-induced positive control (DMEM w/0% FBS and 1 ng/ml TGFb1), 3) ASA (DMEM w/0% FBS and 100 to 3200 μg/ml ASA), and 4) TGFb1+ASA (DMEM w/0% FBS, 1 ng/ml TGFb1 and 100 to 3200 μg/ml ASA). Relative concentration of formazan was compared between treatment groups by measuring the optical density at 570 nm and normalizing to vehicle control (N=6, n=4, *p<0.05, **p<0.01, ****p<0.0001).

FIG. 15B is a graph of the relative metabolic activity of HTCFs (grey bars) and TGFb1-induced HTCFs (black bars) when exposed to different concentrations of APHS. HTCFs were cultured in monolayer and treated with one of the following experimental treatments for 48 hrs: 1) vehicle control (DMEM w/0% FBS), 2) TGFb1-induced positive control (DMEM w/0% FBS and 1 ng/ml TGFb1), 3) APHS (DMEM w/0% FBS and 4 to 16 μg/ml APHS), and 4) TGFb1+APHS (DMEM w/0% FBS, 1 ng/ml TGFb1 and 4 to 16 μg/ml APHS). Relative concentration of formazan was compared between treatment groups by measuring the optical density at 570 nm and normalizing to vehicle control (N=4, n=4, *p<0.05, **p<0.01, ***p<0.001).

FIG. 16A is a graph of the relative expression of proteins: αSMA, MMP9, PPARγ to that of GAPDH after the indicated experimental treatments. HTCFs were cultured in monolayer and exposed to one of five experimental treatments for 48 hrs: 1) vehicle control (DMEM w/0% FBS), 2) AHGFs (DMEM w/0% FBS and 2 ng/ml each of TGFB1, TGFB2, VEGF, CCN2), and 3) AHGFs+ASA100 (DMEM w/0% FBS, 2 ng/ml each of TGFB1, TGFB2, VEGF, CCN2, and 100 μg/ml ASA), and 4) AHGFs+ASA400 (DMEM w/0% FBS, 2 ng/ml each of TGFB1, TGFB2, VEGF, CCN2, and 400 μg/ml ASA), and 5) AHGFs+ASA1600 (DMEM w/0% FBS, 2 ng/ml each of TGFB1, TGFB2, VEGF, CCN2, and 1600 μg/ml ASA). After 48 hrs cellular protein lysate was assessed by western blot and probed with primary antibodies for αSMA, MMP9, PPARγ and GAPDH. Protein expression was first standardized to the expression level of GAPDH and then normalized to the expression level of the AHGFs treatment group. (N=4, ****p<0.001).

FIG. 16B is a representative western blot image of cellular protein lysate. HTCFs were cultured in monolayer and exposed to one of five experimental treatments for 48 hrs: 1) vehicle control (DMEM w/0% FBS), 2) AHGFs (DMEM w/0% FBS and 2 ng/ml each of TGFB1, TGFB2, VEGF, CCN2), and 3) AHGFs+ASA100 (DMEM w/0% FBS, 2 ng/ml each of TGFB1, TGFB2, VEGF, CCN2, and 100 μg/ml ASA), and 4) AHGFs+ASA400 (DMEM w/0% FBS, 2 ng/ml each of TGFB1, TGFB2, VEGF, CCN2, and 400 μg/ml ASA), and 5) AHGFs+ASA1600 (DMEM w/0% FBS, 2 ng/ml each of TGFB1, TGFB2, VEGF, CCN2, and 1600 μg/ml ASA). After 48 hrs cellular protein lysate was assessed by western blot and probed with primary antibodies for αSMA and GAPDH.

FIG. 16C is a representative western blot image of cellular protein lysate. HTCFs were cultured in monolayer and exposed to one of five experimental treatments for 48 hrs: 1) vehicle control (DMEM w/0% FBS), 2) AHGFs (DMEM w/0% FBS and 2 ng/ml each of TGFB1, TGFB2, VEGF, CCN2), and 3) AHGFs+ASA100 (DMEM w/0% FBS, 2 ng/ml each of TGFB1, TGFB2, VEGF, CCN2, and 100 μg/ml ASA), and 4) AHGFs+ASA400 (DMEM w/0% FBS, 2 ng/ml each of TGFB1, TGFB2, VEGF, CCN2, and 400 μg/ml ASA), and 5) AHGFs+ASA1600 (DMEM w/0% FBS, 2 ng/ml each of TGFB1, TGFB2, VEGF, CCN2, and 1600 μg/ml ASA). After 48 hrs cellular protein lysate was assessed by western blot and probed with primary antibodies for MMP9 and GAPDH.

FIG. 16D is a representative western blot image of cellular protein lysate. HTCFs were cultured in monolayer and exposed to one of five experimental treatments for 48 hrs: 1) vehicle control (DMEM w/0% FBS), and 2) AHGFs (DMEM w/0% FBS and 2 ng/ml each of TGFB1, TGFB2, VEGF, CCN2), and 3) AHGFs+ASA100 (DMEM w/0% FBS, 2 ng/ml each of TGFB1, TGFB2, VEGF, CCN2, and 100 μg/ml ASA), and 4) AHGFs+ASA400 (DMEM w/0% FBS, 2 ng/ml each of TGFB1, TGFB2, VEGF, CCN2, and 400 μg/ml ASA), and 5) AHGFs+ASA1600 (DMEM w/0% FBS, 2 ng/ml each of TGFB1, TGFB2, VEGF, CCN2, and 1600 μg/ml ASA). After 48 hrs cellular protein lysate was assessed by western blot and probed with primary antibodies for PPARγ and GAPDH.

FIG. 17A is a graph of the relative expression of collagen 1 to that of GAPDH after the indicated experimental treatments. HTCFs were cultured in monolayer and exposed to one of ten experimental treatments for 48 hrs: 1) vehicle control (DMEM w/0% FBS), 2) AHGFs (DMEM w/0% FBS and 2 ng/ml each of TGFB1, TGFB2, VEGF, CCN2), and 3) AHGFs/Gent250 (DMEM w/0% FBS, 2 ng/ml each of TGFB1, TGFB2, VEGF, CCN2 and 250 μg/ml gentamicin), and 4) AHGFs/Gent500 (DMEM w/0% FBS, 2 ng/ml each of TGFB1, TGFB2, VEGF, CCN2 and 500 μg/ml gentamicin), and 5) AHGFs/ASA500 (DMEM w/0% FBS, 2 ng/ml each of TGFB1, TGFB2, VEGF, CCN2, and 500 μg/ml ASA), and 6) AHGFs/ASA500/Gent250 (DMEM w/0% FBS, 2 ng/ml each of TGFB1, TGFB2, VEGF, CCN2, 500 μg/ml ASA and 250 μg/ml gentamicin), and 7) AHGFs/ASA1000 (DMEM w/0% FBS, 2 ng/ml each of TGFB1, TGFB2, VEGF, CCN2 and 1000 μg/ml ASA), and 8) AHGFs/ASA1000/Gent500 (DMEM w/0% FBS, 2 ng/ml each of TGFB1, TGFB2, VEGF, CCN2, 1000 μg/ml ASA, and 500 μg/ml gentamicin), and 9) AHGFs/APHS12 (DMEM w/0% FBS, 2 ng/ml each of TGFB1, TGFB2, VEGF, CCN2 and 12 μg/ml o-(acetoxyphenyl)hept-2-ynyl sulfide (APHS), and 10) AHGFs/APHS12/Gent250 (DMEM w/0% FBS, 2 ng/ml each of TGFB1, TGFB2, VEGF, CCN2, 12 μg/ml o-(acetoxyphenyl)hept-2-ynyl sulfide (APHS) and 250 μg/ml gentamicin). After 48 hrs cellular protein lysate was assessed by western blot and probed with primary antibodies for collagen 1 and GAPDH. Protein expression was first standardized to the expression level of GAPDH and then normalized to the expression level of the AHGFs treatment group. (N=5, p values are indicated above bars for the representative groups vs. the AHGFs group).

FIG. 17B is a graph of the relative expression of αSMA to that of GAPDH after the indicated experimental treatments. HTCFs were cultured in monolayer and exposed to one of ten experimental treatments for 48 hrs: 1) vehicle control (DMEM w/0% FBS), 2) AHGFs (DMEM w/0% FBS and 2 ng/ml each of TGFB1, TGFB2, VEGF, CCN2), and 3) AHGFs/Gent250 (DMEM w/0% FBS, 2 ng/ml each of TGFB1, TGFB2, VEGF, CCN2 and 250 μg/ml gentamicin), and 4) AHGFs/Gent500 (DMEM w/0% FBS, 2 ng/ml each of TGFB1, TGFB2, VEGF, CCN2 and 500 μg/ml gentamicin), and 5) AHGFs/ASA500 (DMEM w/0% FBS, 2 ng/ml each of TGFB1, TGFB2, VEGF, CCN2, and 500 μg/ml ASA), and 6) AHGFs/ASA500/Gent250 (DMEM w/0% FBS, 2 ng/ml each of TGFB1, TGFB2, VEGF, CCN2, 500 μg/ml ASA and 250 μg/ml gentamicin), and 7) AHGFs/ASA1000 (DMEM w/0% FBS, 2 ng/ml each of TGFB1, TGFB2, VEGF, CCN2 and 1000 μg/ml ASA), and 8) AHGFs/ASA1000/Gent500 (DMEM w/0% FBS, 2 ng/ml each of TGFB1, TGFB2, VEGF, CCN2, 1000 μg/ml ASA, and 500 μg/ml gentamicin), and 9) AHGFs/APHS12 (DMEM w/0% FBS, 2 ng/ml each of TGFB1, TGFB2, VEGF, CCN2 and 12 μg/ml o-(acetoxyphenyl)hept-2-ynyl sulfide (APHS), and 10) AHGFs/APHS12/Gent250 (DMEM w/0% FBS, 2 ng/ml each of TGFB1, TGFB2, VEGF, CCN2, 12 μg/ml o-(acetoxyphenyl)hept-2-ynyl sulfide (APHS) and 250 μg/ml gentamicin). After 48 hrs cellular protein lysate was assessed by western blot and probed with primary antibodies for αSMA and GAPDH. Protein expression was first standardized to the expression level of GAPDH and then normalized to the expression level of the AHGFs treatment group. (N=5, p values are indicated above bars for the representative groups vs. the AHGFs group).

FIG. 17C are representative western blot images of cellular protein lysate collected from two different patient cell lines. HTCFs were cultured in monolayer and exposed to one of ten experimental treatments for 48 hrs: 1) vehicle control (DMEM w/0% FBS), 2) AHGFs (DMEM w/0% FBS and 2 ng/ml each of TGFB1, TGFB2, VEGF, CCN2), and 3) AHGFs/Gent250 (DMEM w/0% FBS, 2 ng/ml each of TGFB1, TGFB2, VEGF, CCN2 and 250 μg/ml gentamicin), and 4) AHGFs/Gent500 (DMEM w/0% FBS, 2 ng/ml each of TGFB1, TGFB2, VEGF, CCN2 and 500 μg/ml gentamicin), and 5) AHGFs/ASA500 (DMEM w/0% FBS, 2 ng/ml each of TGFB1, TGFB2, VEGF, CCN2, and 500 μg/ml ASA), and 6) AHGFs/ASA500/Gent250 (DMEM w/0% FBS, 2 ng/ml each of TGFB1, TGFB2, VEGF, CCN2, 500 μg/ml ASA and 250 μg/ml gentamicin), and 7) AHGFs/ASA1000 (DMEM w/0% FBS, 2 ng/ml each of TGFB1, TGFB2, VEGF, CCN2 and 1000 μg/ml ASA), and 8) AHGFs/ASA1000/Gent500 (DMEM w/0% FBS, 2 ng/ml each of TGFB1, TGFB2, VEGF, CCN2, 1000 μg/ml ASA, and 500 μg/ml gentamicin), and 9) AHGFs/APHS12 (DMEM w/0% FBS, 2 ng/ml each of TGFB1, TGFB2, VEGF, CCN2 and 12 μg/ml o-(acetoxyphenyl)hept-2-ynyl sulfide (APHS), and 10) AHGFs/APHS12/Gent250 (DMEM w/0% FBS, 2 ng/ml each of TGFB1, TGFB2, VEGF, CCN2, 12 μg/ml o-(acetoxyphenyl)hept-2-ynyl sulfide (APHS) and 250 μg/ml gentamicin). After 48 hrs exposure cellular protein lysate was assessed by western blot and probed with primary antibodies for collagen 1, αSMA and GAPDH.

FIG. 18A is a graph of the relative expression of phosphorylated (p)SMAD2/3 to that of GAPDH after the indicated experimental treatments. HTCFs were cultured in monolayer and exposed to one of nine experimental treatments for 48 hrs: 1) vehicle control (DMEM w/0% FBS), 2) AHGFs (DMEM w/0% FBS and 2 ng/ml each of TGFB1, TGFB2, VEGF, CCN2), and 3) AHGFs/Gent500 (DMEM w/0% FBS, 2 ng/ml each of TGFB1, TGFB2, VEGF, CCN2 and 500 μg/ml gentamicin), and 4) AHGFs/ASA500 (DMEM w/0% FBS, 2 ng/ml each of TGFB1, TGFB2, VEGF, CCN2, and 500 μg/ml ASA), and 5) AHGFs/ASA500/Gent250 (DMEM w/0% FBS, 2 ng/ml each of TGFB1, TGFB2, VEGF, CCN2, 500 μg/ml ASA and 250 μg/ml gentamicin), and 6) AHGFs/ASA1000 (DMEM w/0% FBS, 2 ng/ml each of TGFB1, TGFB2, VEGF, CCN2 and 1000 μg/ml ASA), and 7) AHGFs/ASA1000/Gent500 (DMEM w/0% FBS, 2 ng/ml each of TGFB1, TGFB2, VEGF, CCN2, 1000 μg/ml ASA, and 500 μg/ml gentamicin), and 8) AHGFs/APHS12 (DMEM w/0% FBS, 2 ng/ml each of TGFB1, TGFB2, VEGF, CCN2 and 12 μg/ml o-(acetoxyphenyl)hept-2-ynyl sulfide (APHS), and 9) AHGFs/APHS12/Gent250 (DMEM w/0% FBS, 2 ng/ml each of TGFB1, TGFB2, VEGF, CCN2, 12 μg/ml o-(acetoxyphenyl)hept-2-ynyl sulfide (APHS) and 250 μg/ml gentamicin). After 48 hrs cellular protein lysate was assessed by western blot and probed with primary antibodies for pSMAD2/3 and GAPDH. Protein expression was first standardized to the expression level of GAPDH and then normalized to the expression level of the AHGFs treatment group. (N=5, p values are indicated above bars for the representative groups vs. the AHGFs group).

FIG. 18B is a graph of the relative expression of non-phosphorylated SMAD2/3 to that of GAPDH after the indicated experimental treatments. HTCFs were cultured in monolayer and exposed to one of nine experimental treatments for 48 hrs: 1) vehicle control (DMEM w/0% FBS), 2) AHGFs (DMEM w/0% FBS and 2 ng/ml each of TGFB1, TGFB2, VEGF, CCN2), and 3) AHGFs/Gent500 (DMEM w/0% FBS, 2 ng/ml each of TGFB1, TGFB2, VEGF, CCN2 and 500 μg/ml gentamicin), and 4) AHGFs/ASA500 (DMEM w/0% FBS, 2 ng/ml each of TGFB1, TGFB2, VEGF, CCN2, and 500 μg/ml ASA), and 5) AHGFs/ASA500/Gent250 (DMEM w/0% FBS, 2 ng/ml each of TGFB1, TGFB2, VEGF, CCN2, 500 μg/ml ASA and 250 μg/ml gentamicin), and 6) AHGFs/ASA1000 (DMEM w/0% FBS, 2 ng/ml each of TGFB1, TGFB2, VEGF, CCN2 and 1000 μg/ml ASA), and 7) AHGFs/ASA1000/Gent500 (DMEM w/0% FBS, 2 ng/ml each of TGFB1, TGFB2, VEGF, CCN2, 1000 μg/ml ASA, and 500 μg/ml gentamicin), and 8) AHGFs/APHS12 (DMEM w/0% FBS, 2 ng/ml each of TGFB1, TGFB2, VEGF, CCN2 and 12 μg/ml o-(acetoxyphenyl)hept-2-ynyl sulfide (APHS), and 9) AHGFs/APHS12/Gent250 (DMEM w/0% FBS, 2 ng/ml each of TGFB1, TGFB2, VEGF, CCN2, 12 μg/ml o-(acetoxyphenyl)hept-2-ynyl sulfide (APHS) and 250 μg/ml gentamicin). After 48 hrs cellular protein lysate was assessed by western blot and probed with primary antibodies for non-phosphorylated SMAD2/3 and GAPDH. Protein expression was first standardized to the expression level of GAPDH and then normalized to the expression level of the AHGFs treatment group. (N=5, p values are indicated above bars for the representative groups vs. the AHGFs group).

FIG. 18C is a graph of the ratio of the relative expression of phosphorylated (p)SMAD2/3 to that of total SMAD2/3 for the indicated experimental treatments. HTCFs were cultured in monolayer and exposed to one of nine experimental treatments for 48 hrs: 1) vehicle control (DMEM w/0% FBS), 2) AHGFs (DMEM w/0% FBS and 2 ng/ml each of TGFB1, TGFB2, VEGF, CCN2), and 3) AHGFs/Gent500 (DMEM w/0% FBS, 2 ng/ml each of TGFB1, TGFB2, VEGF, CCN2 and 500 μg/ml gentamicin), and 4) AHGFs/ASA500 (DMEM w/0% FBS, 2 ng/ml each of TGFB1, TGFB2, VEGF, CCN2, and 500 μg/ml ASA), and 5) AHGFs/ASA500/Gent250 (DMEM w/0% FBS, 2 ng/ml each of TGFB1, TGFB2, VEGF, CCN2, 500 μg/ml ASA and 250 μg/ml gentamicin), and 6) AHGFs/ASA1000 (DMEM w/0% FBS, 2 ng/ml each of TGFB1, TGFB2, VEGF, CCN2 and 1000 μg/ml ASA), and 7) AHGFs/ASA1000/Gent500 (DMEM w/0% FBS, 2 ng/ml each of TGFB1, TGFB2, VEGF, CCN2, 1000 μg/ml ASA, and 500 μg/ml gentamicin), and 8) AHGFs/APHS12 (DMEM w/0% FBS, 2 ng/ml each of TGFB1, TGFB2, VEGF, CCN2 and 12 μg/ml o-(acetoxyphenyl)hept-2-ynyl sulfide (APHS), and 9) AHGFs/APHS12/Gent250 (DMEM w/0% FBS, 2 ng/ml each of TGFB1, TGFB2, VEGF, CCN2, 12 μg/ml o-(acetoxyphenyl)hept-2-ynyl sulfide (APHS) and 250 μg/ml gentamicin). After 48 hrs cellular protein lysate was assessed by western blot and probed with primary antibodies for phosphorylated (p)SMAD2/3, non-phosphorylated SMAD2/3 and GAPDH. Protein expression was first standardized to the expression level of GAPDH and then normalized to the expression level of the AHGFs treatment group. Relative expression values for (p)SMAD2/3 were then divided by the sum of the relative expression values for (p)SMAD2/3+SMAD2/3. (N=5, p values are indicated above bars for the representative groups vs. the AHGFs group).

FIG. 18D are representative western blot images of cellular protein lysate collected from two different patient cell lines. HTCFs were cultured in monolayer and exposed to one of nine experimental treatments for 48 hrs: 1) vehicle control (DMEM w/0% FBS), 2) AHGFs (DMEM w/0% FBS and 2 ng/ml each of TGFB1, TGFB2, VEGF, CCN2), and 3) AHGFs/Gent500 (DMEM w/0% FBS, 2 ng/ml each of TGFB1, TGFB2, VEGF, CCN2 and 500 μg/ml gentamicin), and 4) AHGFs/ASA500 (DMEM w/0% FBS, 2 ng/ml each of TGFB1, TGFB2, VEGF, CCN2, and 500 μg/ml ASA), and 5) AHGFs/ASA500/Gent250 (DMEM w/0% FBS, 2 ng/ml each of TGFB1, TGFB2, VEGF, CCN2, 500 μg/ml ASA and 250 μg/ml gentamicin), and 6) AHGFs/ASA1000 (DMEM w/0% FBS, 2 ng/ml each of TGFB1, TGFB2, VEGF, CCN2 and 1000 μg/ml ASA), and 7) AHGFs/ASA1000/Gent500 (DMEM w/0% FBS, 2 ng/ml each of TGFB1, TGFB2, VEGF, CCN2, 1000 μg/ml ASA, and 500 μg/ml gentamicin), and 8) AHGFs/APHS12 (DMEM w/0% FBS, 2 ng/ml each of TGFB1, TGFB2, VEGF, CCN2 and 12 μg/ml o-(acetoxyphenyl)hept-2-ynyl sulfide (APHS), and 9) AHGFs/APHS12/Gent250 (DMEM w/0% FBS, 2 ng/ml each of TGFB1, TGFB2, VEGF, CCN2, 12 μg/ml o-(acetoxyphenyl)hept-2-ynyl sulfide (APHS) and 250 μg/ml gentamicin). After 48 hrs exposure cellular protein lysate was assessed by western blot and probed with primary antibodies for phosphorylated (p)SMAD2/3, non-phosphorylated SMAD2/3 and GAPDH.

FIG. 19A is a graph of the relative expression of αSMA to that of GAPDH after the indicated experimental treatments. HTCFs were cultured in monolayer and exposed to one of 14 experimental treatments for 48 hrs: 1) vehicle control (DMEM w/0% FBS), and 2) AHGFs (DMEM w/0% FBS and 2 ng/ml each of TGFB1, TGFB2, VEGF, CCN2), and 3-5) AHGFs/5-HETE (DMEM w/0% FBS, 2 ng/ml each of TGFB1, TGFB2, VEGF, CCN2, and 10, 100, or 1000 nM of 5-hydroxyeicosatetraenoic acid), and 6-8) AHGFs/11-HETE (DMEM w/0% FBS, 2 ng/ml each of TGFB1, TGFB2, VEGF, CCN2, and 10, 100, or 1000 nM of 11-hydroxyeicosatetraenoic acid), and 9-11) AHGFs/15-HETE (DMEM w/0% FBS, 2 ng/ml each of TGFB1, TGFB2, VEGF, CCN2, and 10, 100, or 1000 nM of 15-hydroxyeicosatetraenoic acid), and 12-14) AHGFs/17-OHDHA (DMEM w/0% FBS, 2 ng/ml each of TGFB1, TGFB2, VEGF, CCN2, and 10, 100, or 1000 nM of 17-hydroxy-docosahexaenoic acid). After 48 hrs cellular protein lysate was assessed by western blot and probed with primary antibodies for αSMA and GAPDH. Protein expression was first standardized to the expression level of GAPDH and then normalized to the expression level of the AHGFs treatment group. (N=6, *p<0.05, ***p<0.001, ****p<0.0001 vs. the AHGFs group).

FIG. 19B are representative western blot images of cellular protein lysate collected from three different patient cell lines. HTCFs were cultured in monolayer and exposed to one of 14 experimental treatments for 48 hrs: 1) vehicle control (DMEM w/0% FBS), and 2) AHGFs (DMEM w/0% FBS and 2 ng/ml each of TGFB1, TGFB2, VEGF, CCN2), and 3-5) AHGFs/5-HETE (DMEM w/0% FBS, 2 ng/ml each of TGFB1, TGFB2, VEGF, CCN2, and 10, 100, or 1000 nM of 5-hydroxyeicosatetraenoic acid), and 6-8) AHGFs/11-HETE (DMEM w/0% FBS, 2 ng/ml each of TGFB1, TGFB2, VEGF, CCN2, and 10, 100, or 1000 nM of 11-hydroxyeicosatetraenoic acid), and 9-11) AHGFs/15-HETE (DMEM w/0% FBS, 2 ng/ml each of TGFB1, TGFB2, VEGF, CCN2, and 10, 100, or 1000 nM of 15-hydroxyeicosatetraenoic acid), and 12-14) AHGFs/17-OHDHA (DMEM w/0% FBS, 2 ng/ml each of TGFB1, TGFB2, VEGF, CCN2, and 10, 100, or 1000 nM of 17-hydroxy-docosahexaenoic acid). After 48 hrs exposure cellular protein lysate was assessed by western blot and probed with primary antibodies for αSMA and GAPDH.

### DETAILED DESCRIPTION OF THE DISCLOSURE

Compositions and methods for treating ocular inflammation and/or fibrosis and/or scarring in a subject are disclosed. That the disclosure may be more readily understood, select terms are defined below.

### Definitions

The terminology used herein is for the purpose of describing particular embodiments only and is not intended to be limiting of example embodiments of the invention. Unless defined otherwise, all technical and scientific terms used herein generally have the same meaning as commonly understood by one of ordinary skill in the art to which this disclosure belongs.

As used herein, the singular forms “a,” “an,” and “the,” are intended to include the plural forms as well, unless the context clearly indicates otherwise.

The phrase “and/or,” as used herein in the specification and in the claims, should be understood to mean “either or both” of the elements so conjoined, i.e., elements that are conjunctively present in some cases and disjunctively present in other cases. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

When the term “about” is used in conjunction with a numerical range, it modifies that range by extending the boundaries above and below those numerical values. In general, the term “about” is used herein to modify a numerical value above and below the stated value by a variance of 20%, 10%, 5%, or 1%. In certain embodiments, the term “about” is used to modify a numerical value above and below the stated value by a variance of 10%. In certain embodiments, the term “about” is used to modify a numerical value above and below the stated value by a variance of 5%. In certain embodiments, the term “about” is used to modify a numerical value above and below the stated value by a variance of 1%.

When a range of values is listed herein, it is intended to encompass each value and sub-range within that range. For example, “1-5 ng” is intended to encompass 1 ng, 2 ng, 3 ng, 4 ng, 5 ng, 1-2 ng, 1-3 ng, 1-4 ng, 1-5 ng, 2-3 ng, 2-4 ng, 2-5 ng, 3-4 ng, 3-5 ng, and 4- 5 ng.

It will be further understood that the terms “comprises,” “comprising,” “includes,” and/or “including,” when used herein, specify the presence of stated features, integers, steps, operations, elements, and/or components, but do not preclude the presence or addition of one or more other features, integers, steps, operations, elements, components, and/or groups thereof.

A “subject” is a vertebrate, preferably a mammal (e.g., a non-human mammal), more preferably a primate and still more preferably a human. Mammals include, but are not limited to, primates, humans, farm animals, sport animals, and pets.

As used herein, “ocular” refers to the eye or to vision. The compositions and methods of the disclosure can be used to treat ocular inflammation and/or fibrosis and/or scarring. In an embodiment, the compounds and methods are used to treat inflammation of the conjunctiva, caused for example, by conjunctivitis, such as allergic conjunctivitis or any other type of conjunctivitis. In an embodiment, the compounds and methods are administered before, during or after ocular surgery. In various embodiments, the ocular surgery comprises: a) manipulation of the conjunctiva and/or Tenons; and/or b) a conjunctival incision or excision; and/or c) implantation of a medical device within or around the eye; and/or 4) a corneal incision. The ocular surgery may be, for example, micro-invasive glaucoma surgery, cataract surgery, retinal detachment repair surgery, strabismus surgery, vitrectomy, pterygium removal, an excisional biopsy, trauma reconstruction, and/or implantation of a stent, valve, implant or shunt within or around the eye.

As used herein, a “COX2 Ser516 acetylating agent” is a molecule that covalently attaches an acetyl group to amino acid residue Ser516 on COX2. In this case, the COX2 Ser516 acetylation can cause the COX2 enzyme to cease or become impaired in producing PGs and gain the ability to generate 5-HETE, 15-HETE, 17-OHDHA and other pro-resolving lipid mediators from AA/DHA/EPA. Examples of COX2 Ser516 acetylating agents are known in the art and include, but are not limited to: acetylsalicylic acid (ASA), o-(acetoxyphenyl)hept-2-ynyl sulfide (APHS) and other 2-acetoxyphenyl alkyl sulfides [46-50].

As used herein, a “cPLA2 agonist” is a molecule that stimulates or increases activity or expression of the enzyme cytosolic phospholipase A2. In embodiments of this disclosure, the cPLA2 agonist may cause greater amounts of arachidonic acid, docosahexaenoic acid and eicosapentaenoic acid to be present. The cPLA2 agonists may or may not have antibiotic properties. Examples of cPLA2 agonists are known in the art and include, but are not limited to: gentamicin [51], mastoparan [52], phospholipase A2 Activating Protein (PLAP) [53], tetrahydrofurandiol [54] or melittin [55] which in principle would either directly stimulate cPLA2 or stimulate an endogenous immune response which involves the activation of cPLA2. In an embodiment, the cPLA2 agonist is not an antibiotic agent. In an embodiment, the cPLA2 agonist is not gentamicin.

As used herein, the terms “treat,” “treatment,” “treating,” “prophylaxis” or “amelioration” refer to therapeutic treatments, wherein the object is to reverse, alleviate, ameliorate, inhibit, prevent, slow down or stop the progression or severity of ocular inflammation and/or fibrosis and/or scarring in a subject. The subject may have inflammation of the conjunctiva, caused, for example, by conjunctivitis, such as, allergic conjunctivitis. The subject may be about to undergo, is undergoing or has undergone ocular surgery. The term “treating” includes reducing or alleviating at least one adverse effect or symptom of ocular inflammation or fibrosis and/or scarring. Treatment is generally “effective” if one or more symptoms or clinical markers of ocular inflammation or fibrosis and/or scarring are reduced. Alternatively, treatment is “effective” if the progression of inflammation or fibrosis and/or scarring is reduced or halted. That is, “treatment” includes not just the improvement of symptoms or markers, but also a cessation of, or at least slowing of, progress or worsening of symptoms compared to what would be expected in the absence of treatment. Beneficial or desired clinical results include, but are not limited to, alleviation of one or more symptom(s), diminishment of extent of disease, stabilized (i.e., not worsening) state of disease, delay or slowing of disease progression, amelioration or palliation of the disease state, remission (whether partial or total), and/or decreased mortality, whether detectable or undetectable. The term “treatment” of a disease also includes providing relief from the symptoms or side-effects of the disease (including palliative treatment).

As used herein, the term “administering,” refers to the placement of a composition as disclosed herein into a subject by a method or route which results in at least partial delivery of the composition at a desired site. Pharmaceutical compositions disclosed herein can be administered by any appropriate route which results in an effective treatment in the subject. In various embodiments, the pharmaceutical compositions disclosed herein may be administered systemically or locally. In an embodiment, the pharmaceutical compositions disclosed herein may be administered locally to the ocular area or directly to the eye (e.g., ocular and/or intraocular administration). In an embodiment, the pharmaceutical compositions disclosed herein may be administered to the eye using thermoresponsive hydrogel-containing polymer microparticles, such as those described in U.S. Patent Application Publication Number US2019/0099365.

As used herein, “resolving ocular inflammation” refers to decreasing ocular inflammation by promoting its resolution. More specifically, resolving ocular inflammation refers to decreasing, inhibiting and/or mitigating ocular inflammation by increasing the amount and/or activity of pro-resolving lipid mediators (e.g., 5-HETE, 15-HETE and/or 17-OHDHA) rather than, or in addition to, decreasing the amount and/or activity of pro-inflammatory mediators (e.g., prostaglandins). In this way, resolving ocular inflammation using the compositions and methods disclosed herein may have the additional benefit of decreasing inflammation more robustly and earlier during the inflammatory response, resulting in less ocular fibrosis and/or scarring compared to traditional anti-inflammatory therapeutic approaches that do not increase the amount and/or activity of pro-resolving lipid mediators.

### Abbreviations

AA: arachidonic acid
AHGFs: aqueous humor growth factors (more specifically, a custom mixture of endogenous inflammation and fibrotic signaling molecules within glaucomatous aqueous humor: IL-1b (1 ng/ml), TNFa (1 ng/ml), IFNγ (1 ng/ml), TGFB1 (1 ng/ml), TGFB2 (4 ng/ml), VEGF (2 ng/ml) and CCN2 (2 ng/ml))
APHS: o-(acetoxyphenyl)hept-2-ynyl sulfide
ASA: acetylsalicylic acid
αSMA: a.k.a. αSMA: alpha smooth muscle actin
CCN2: a.k.a. CTGF: connective tissue growth factor
CI: confidence interval
Col 1: collagen 1
COX: cyclooxygenase
COX1: cyclooxygenase 1
COX2: cyclooxygenase 2
cPLA2: cytosolic phospholipase A2
CytoM1: Cytokine Mix 1 (more specifically, a custom mixture of endogenous inflammatory cytokines that are naturally released as part of the endogenous activation of an inflammatory response: IL-1b (1 ng/ml), TNFa (1 ng/ml) and IFNγ (1 ng/ml))
DAPI: 4′,6-Diamidino-2-Phenylindole, Dihydrochloride
DHA: docosahexaenoic acid
DMEM: Dulbecco's Modified Eagle's Medium
EPA: eicosapentaenoic acid
FBS: fetal bovine serum
FDA: fluorescein diacetate
Gent: gentamicin
18-HEPE: 18-hydroxyeicosapentaenoic acid
15-HETE: 15-hydroxyeicosatetraenoic acid
5-HETE: 5-hydroxyeicosatetraenoic acid
HTCF(s): human Tenon's capsule fibroblast(s)
IFNγ: interferon gamma
IL-1b: interleukin 1 beta
IOP: intraocular pressure
k6PGF1a/kPGF1a/PGF2a: 6-keto-prostaglandin F1 alpha
LC-MS/MS: liquid chromatography tandem mass spectrometry
LOX: lipoxygenase
Mel: melittin
MMC: mitomycin C
MMP: matrix metalloproteinase
MMP9: matrix metalloproteinase 9
MTT: 3-[4,5-dimethylthiazol-2-yl]-2,5-diphenyltetrazolium bromide
ns: not significant (as determined by a statistical test)
NSAID: non-steroidal anti-inflammatory drug
17-OHDHA: 17-hydroxy-docosahexaenoic acid
PG: prostaglandin
PGD2: prostaglandin D2
PGE2: prostaglandin E2
PGI2: prostaglandin 12
PI: propidium iodide
PLA2: phospholipase A2
PLAP: phospholipase A2 activating protein
PPARγ: peroxisome proliferator activator receptor gamma
PUFA: polyunsaturated fatty acid
Ser: serine
SMAD2/3: un-phosphorylated version of the protein known as “suppressor of mothers against decapentaplegic”
(p)SMAD2/3: phosphorylated version SMAD2/3
(t)SMAD2/3: total (phosphorylated and unphosphorylated) SMAD2/3
TGFB1: transforming growth factor beta 1
TGFB2: transforming growth factor beta 2
TNFa: tumor necrosis factor alpha
VC: vehicle control
VEGF: vascular endothelial growth factor

### GENERAL DESCRIPTION

The inventors have surprisingly discovered that the compositions described herein modulate inflammatory and wound healing phenomena (prostaglandin production, SPM production, collagen production, αSMA expression, MMP expression, PPARγ expression, SMAD2/3 phosphorylation, SMAD2/3 expression, collagen contraction, collagen remodelling, fibroblast/myofibroblast transdifferentiation, metabolic activity and proliferation) in a way that is therapeutically desirable in ophthalmic surgery and ocular inflammation. While not wishing to be bound or limited by any theories, the compositions described herein may modify the cell's endogenous biosynthetic pathways in at least two ways: (1) using a cPLA2 agonist, such that the overall biosynthetic activity of the cPLA2 pathway is increased and (2) using a COX2 Ser516 acetylating agent (i.e. ASA or APHS) to acetylate Ser516 on COX2, such that the cPLA2-COX2 pathway's net production of PGs is impaired and replaced by the production of acetyl-COX2 products. This production of acetyl-COX2 products contributes to resolving acute or chronic inflammatory insults and the subsequent mitigation of unwanted HTCF scarring activity. The result is an impairment or inhibition of inflammation induced myofibroblast transdifferentiation and ultimately a reduction in HTCF-mediated collagen contraction and collagen remodelling. Thus, ocular anatomy can be preserved in the face of inflammatory insults.

### Compositions and Methods of Treatment

In certain embodiments, the disclosure provides compositions for the treatment or prophylaxis of ocular inflammation and/or fibrosis and/or scarring in a subject, or to preserve (at least in part) and/or rescue (at least in part) ocular tissue from damage caused by inflammation and/or fibrosis and/or scarring. In one embodiment, the ocular inflammation and/or fibrosis and/or scarring occurs as a result of ocular surgery. In another embodiment the ocular inflammation and/or fibrosis and/or scarring is caused by conjunctivitis. In an embodiment, the conjunctivitis is allergic conjunctivitis.

In one embodiment, a therapeutically effective amount of a COX2 Ser516 acetylating agent such as ASA or APHS is administered to the subject. In an embodiment, the administration is local administration (e.g., to the ocular area).

In another embodiment, a combination of therapeutically effective amounts of a COX2 Ser516 acetylating agent and a cPLA2 agonist such as gentamicin, mastoparan, phospholipase A2 activating protein (PLAP), tetrahydrofurandiol or melittin are administered to the subject. In this embodiment, the COX2 Ser516 acetylating agent and the cPLA2 agonist can be formulated into a single composition or may each be formulated separately with a pharmaceutically acceptable carrier. When formulated separately, the two compositions may be administered to the subject sequentially, in any order, or each composition may be administered simultaneously to the subject.

The COX2 Ser516 acetylating agent, alone or in combination with a cPLA2 agonist described herein can be provided in a pharmaceutical composition. Depending on the intended mode of administration, the pharmaceutical composition can be in the form of solid, semi-solid or liquid dosage forms, such as, for example, capsules, powders, liquids, or suspensions, preferably in unit dosage form suitable for single administration of a precise dosage. In one embodiment, the pharmaceutical composition is formulated for local delivery to the ocular tissue, for example, by topical application or local injection of a liquid formulation or an ointment. In certain embodiments, the composition is a depot injection (e.g., a solid or semi-solid injectable formulation that remains positioned within the desired tissue local) coated on or within an implanted medical device configured to release the composition slowly over time. In an embodiment, the depot injection or the composition coated on a medical device is formulated to deliver the composition to the tissue in need thereof over a period of about three months to two years. In one embodiment, the depot injection or the composition coated on a medical device is formulated to deliver the composition to the tissue in need thereof over a period of about six months.

The pharmaceutical compositions include a therapeutically effective amount of a COX2 Ser516 acetylating agent, alone or in combination with a cPLA2 agonist in combination with a pharmaceutically acceptable carrier and, in addition, may include other medicinal agents, pharmaceutical agents, carriers, or diluents. By “pharmaceutically acceptable” is meant a material that is not biologically or otherwise undesirable, which can be administered to an individual along with the selected agent without causing unacceptable biological effects or interacting in a deleterious manner with the other components of the pharmaceutical composition in which it is contained.

As used herein, the term “carrier” encompasses any excipient, diluent, filler, salt, buffer, stabilizer, solubilizer, lipid, stabilizer, or other material well known in the art for use in pharmaceutical formulations. The choice of a carrier for use in a composition will depend upon the intended route of administration for the composition. The preparation of pharmaceutically acceptable carriers and formulations containing these materials is described in, e.g., Remington's Pharmaceutical Sciences, 21st Edition, ed. University of the Sciences in Philadelphia, Lippincott, Williams & Wilkins, Philadelphia Pa., 2005. Examples of physiologically acceptable carriers include buffers such as phosphate buffers, citrate buffer, and buffers with other organic acids; antioxidants including ascorbic acid; low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, arginine or lysine; monosaccharides, disaccharides, and other carbohydrates including glucose, mannose, or dextrins; chelating agents such as EDTA; sugar alcohols such as mannitol or sorbitol; salt-forming counterions such as sodium; and/or nonionic surfactants such as TWEEN™ (ICI, Inc.; Bridgewater, N.J.), polyethylene glycol (PEG), and PLURONICS™ (BASF; Florham Park, N.J.).

The compositions and methods provided herein are applicable to a variety of ocular tissues (e.g., of a subject). For example, compositions and methods can be used to treat inflammation of the conjunctiva, caused for example, by conjunctivitis (e.g., allergic conjunctivitis) or any other type of conjunctivitis. The compositions and methods can be administered before, during and/or after ocular surgery, for example, surgery involving manipulation of the conjunctiva and/or Tenons; b) a conjunctival incision or excision; c) implantation of a medical device within or around the eye; and/or 4) a corneal incision. Examples of ocular surgery include, but are not limited to: micro-invasive glaucoma surgery, cataract surgery, retinal detachment repair surgery, strabismus surgery, vitrectomy, pterygium removal, an excisional biopsy, trauma reconstruction, or implantation of a stent, valve, implant or shunt within or around the eye.

Administration can be carried out using therapeutically effective amounts of the agents described herein for periods of time effective to treat inflammation and/or fibrosis and/or scarring. The effective amount may be determined by one of ordinary skill in the art and includes exemplary dosage amounts for a mammal of from about 0.5 to about 700 mg/kg of body weight of active compound per day, which may be administered in a single dose or in the form of individual divided doses, such as from 1 to 4 times per day. Alternatively, the dosage amount can be from about 0.5 to about 200 mg/kg of body weight of active compound per day, about 0.5 to about 150 mg/kg of body weight of active compound per day, about 0.5 to 100 mg/kg of body weight of active compound per day, about 0.5 to about 75 mg/kg of body weight of active compound per day, about 0.5 to about 50 mg/kg of body weight of active compound per day, about 0.5 to about 25 mg/kg of body weight of active compound per day, about 1 to about 20 mg/kg of body weight of active compound per day, about 1 to about 10 mg/kg of body weight of active compound per day, about 20 mg/kg of body weight of active compound per day, about 10 mg/kg of body weight of active compound per day, or about 5 mg/kg of body weight of active compound per day. Administration can also be carried out in multiple doses, for example, hourly, daily, weekly, monthly etc. In certain embodiments, the composition is a depot injection (e.g. a solid or semi-solid injectable formulation that remains positioned within the desired tissue local) coated on or within an implanted medical device configured to release the composition slowly over time. In an embodiment, the depot injection or the composition coated on a medical device is formulated to deliver the composition to the tissue in need thereof over a period of about three months to two years. In one embodiment, the depot injection or the composition coated on a medical device is formulated to deliver the composition to the tissue in need thereof over a period of about six months.

According to the methods taught herein, the subject is administered an effective amount of a COX2 Ser516 acetylating agent, alone or in combination with a cPLA2 agonist. The terms “effective amount” and “effective dosage” are used interchangeably. The term effective amount is defined as any amount necessary to produce a desired physiologic response, such as decreased ocular inflammation, decreased ocular fibrosis and/or scarring, decreased collagen contraction and/or remodelling and decreased fibroblast cellular proliferation. Effective amounts and schedules for administering the agent may be determined empirically, and making such determinations is within the skill in the art. The dosage ranges for administration are those large enough to produce the desired effect in which one or more symptoms of the disease or disorder are affected (e.g., reduced or delayed). The dosage should not be so large as to cause substantial adverse side effects, such as unwanted cross-reactions, anaphylactic reactions, and the like. Generally, the dosage will vary with the activity of the specific compound employed, the metabolic stability and length of action of that compound, the species, age, body weight, general health, sex and diet of the subject, the mode and time of administration, rate of excretion, drug combination, and severity of the particular condition and can be determined by one of skill in the art. The dosage can be adjusted by the individual physician in the event of any contraindications. Dosages can vary, and can be administered in one or more dose administrations daily, for one or several days. Guidance can be found in the literature for appropriate dosages for given classes of pharmaceutical products. Effective doses can also be extrapolated from dose-response curves derived from in vitro or animal model test systems.

Various concentrations of a COX2 Ser516 acetylating agent, alone or in combination with a cPLA2 agonist, can be used to treat human or animal subjects before, during or after the subject undergoes ocular inflammation and/or fibrosis and/or scarring. For example, the subject may be treated with a COX2 Ser516 acetylating agent, alone or in combination with a cPLA2 agonist before, during or after ocular surgery. Preferably, the ocular tissue is treated locally. Methods for administering the compositions disclosed herein are known to those skilled in the art. Administration of the compositions disclosed herein, may be carried out at various doses over various time periods.

In an embodiment, the cPLA2 agonist and mechanism of combined delivery with the COX2 Ser516 acetylating agent used is such that, in such amounts that, the duration of action and tissue bioavailability of the cPLA2 agonist does not exceed that of a COX2 Ser516 acetylating agent. The duration of bioavailability, potency and dose of the cPLA2 agonist relative to a COX2 Ser516 acetylating agent can be adjusted by those skilled in the art such to produce preparations of the composition with varying strength if desired.

In an embodiment, the compositions and methods treat an ocular tissue by preserving and/or improving ocular tissue function. In other embodiments, the compositions and methods treat an ocular tissue by reducing ocular collagen contraction and/or remodelling or by reducing ocular fibroblast cellular proliferation in the subject. Methods for assessing ocular tissue function, including function of the conjunctiva and/or Tenons, as well as ocular collagen contraction and/or remodelling or by reducing ocular fibroblast cellular proliferation are known in the art and are provided herein. Results of ocular tissue treatment as described herein may be measured in a variety of ways, such as, for example, by a functional assay known in the art (i.e., to determine one or more indicators of ocular tissue function), or a molecular assay known in the art (i.e., to determine one or more molecular feature of the tissue).

Compositions comprising a COX2 Ser516 acetylating agent, alone or with a cPLA2 agonist may be used to decrease ocular inflammation, to decrease ocular fibrosis and/or scarring, to decrease ocular collagen contraction and/or remodelling, and/or to decrease ocular fibroblast proliferation in a subject. In certain embodiments, ocular inflammation, ocular fibrosis and/or scarring, ocular collagen contraction and/or remodeling and/or ocular fibroblast proliferation is decreased relative to an ocular tissue that does not receive the composition. In an embodiment, ocular inflammation, ocular fibrosis and/or scarring, ocular collagen contraction and/or remodeling and/or ocular fibroblast proliferation is decreased by about 5%, 10%, 15%, 20%, 25%, 30%, 35%, 40%, 45%, 50%, 55%, 60%, 65%, 70%, 75%, 80%, 85, 90%, or 95% relative to an ocular tissue that does not receive the composition.

Compositions comprising a COX2 Ser516 acetylating agent, alone or with a cPLA2 agonist may be used to decrease ocular fibrosis and/or scarring in a subject. In certain embodiments, ocular inflammation is decreased relative to an ocular tissue that does not receive the composition. In an embodiment, ocular inflammation, ocular fibrosis and/or scarring, ocular collagen contraction and/or remodeling and/or ocular fibroblast proliferation is decreased by about 5%, 10%, 15%, 20%, 25%, 30%, 35%, 40%, 45%, 50%, 55%, 60%, 65%, 70%, 75%, 80%, 85, 90%, or 95% relative to an ocular tissue that does not receive the composition.

Compositions comprising a COX2 Ser516 acetylating agent, alone or with a cPLA2 agonist may be used to decrease ocular prostaglandin production in a subject. In certain embodiments, ocular prostaglandin production is decreased relative to an ocular tissue that does not receive the composition. In an embodiment, ocular prostaglandin production, inflammation, ocular fibrosis and/or scarring, ocular collagen contraction and/or remodeling and/or ocular fibroblast proliferation is decreased by about 5%, 10%, 15%, 20%, 25%, 30%, 35%, 40%, 45%, 50%, 55%, 60%, 65%, 70%, 75%, 80%, 85, 90%, or 95% relative to an ocular tissue that does not receive the composition.

Compositions comprising a COX2 Ser516 acetylating agent, alone or with a cPLA2 agonist may be used to increase ocular production of 5-HETE, 15-HETE and 17-OHDHA pro-resolving lipid mediators in a subject. In certain embodiments, ocular production of 5-HETE, 15-HETE and 17-OHDHA pro-resolving lipid mediators is increased relative to an ocular tissue that does not receive the composition. In an embodiment, ocular prostaglandin production, inflammation, ocular fibrosis and/or scarring, ocular collagen contraction and/or remodeling and/or ocular fibroblast proliferation is decreased by about 5%, 10%, 15%, 20%, 25%, 30%, 35%, 40%, 45%, 50%, 55%, 60%, 65%, 70%, 75%, 80%, 85, 90%, or 95%; and the ocular production of pro-resolving lipid mediators 5-HETE, 15-HETE and 17-OHDHA is increased by about 5%, 10%, 15%, 20%, 25%, 30%, 35%, 40%, 45%, 50%, 55%, 60%, 65%, 70%, 75%, 80%, 85, 90%, 95%, 100%, 500%, 1000%, or 10,000% relative to an ocular tissue that does not receive the composition.

Compositions comprising a COX2 Ser516 acetylating agent, alone or with a cPLA2 agonist may be used to decrease the expression of collagen by ocular fibroblasts in a subject. In certain embodiments, the expression of collagen by ocular fibroblasts is decreased relative to an ocular tissue that does not receive the composition. In an embodiment, the expression of collagen by ocular fibroblasts, ocular prostaglandin production, inflammation, ocular fibrosis and/or scarring, ocular collagen contraction and/or remodeling and/or ocular fibroblast proliferation is decreased by about 5%, 10%, 15%, 20%, 25%, 30%, 35%, 40%, 45%, 50%, 55%, 60%, 65%, 70%, 75%, 80%, 85, 90%, or 95%; and the ocular production of pro-resolving lipid mediators 5-HETE, 15-HETE and 17-OHDHA is increased by about 5%, 10%, 15%, 20%, 25%, 30%, 35%, 40%, 45%, 50%, 55%, 60%, 65%, 70%, 75%, 80%, 85, 90%, 95%, 100%, 500%, 1000%, or 10,000% relative to an ocular tissue that does not receive the composition.

Compositions comprising a COX2 Ser516 acetylating agent, alone or with a cPLA2 agonist may be used to decrease the expression of matrix metalloproteinases (MMPs) by ocular fibroblasts in a subject. In certain embodiments, the expression of MMPs by ocular fibroblasts is decreased relative to an ocular tissue that does not receive the composition. In an embodiment, the expression of MMPs and collagen by ocular fibroblasts, ocular prostaglandin production, inflammation, ocular fibrosis and/or scarring, ocular collagen contraction and/or remodeling and/or ocular fibroblast proliferation is decreased by about 5%, 10%, 15%, 20%, 25%, 30%, 35%, 40%, 45%, 50%, 55%, 60%, 65%, 70%, 75%, 80%, 85, 90%, or 95%; and the ocular production of pro-resolving lipid mediators 5-HETE, 15-HETE and 17-OHDHA is increased by about 5%, 10%, 15%, 20%, 25%, 30%, 35%, 40%, 45%, 50%, 55%, 60%, 65%, 70%, 75%, 80%, 85, 90%, 95%, 100%, 500%, 1000%, or 10,000% relative to an ocular tissue that does not receive the composition.

Compositions comprising a COX2 Ser516 acetylating agent, alone or with a cPLA2 agonist may be used to increase the expression of peroxisome proliferator-activated receptor gamma (PPARγ) by ocular fibroblasts in a subject. In certain embodiments, the expression of PPARγ by ocular fibroblasts is increased relative to an ocular tissue that does not receive the composition. In an embodiment, the expression of MMPs and collagen by ocular fibroblasts, ocular prostaglandin production, inflammation, ocular fibrosis and/or scarring, ocular collagen contraction and/or remodeling and/or ocular fibroblast proliferation is decreased by about 5%, 10%, 15%, 20%, 25%, 30%, 35%, 40%, 45%, 50%, 55%, 60%, 65%, 70%, 75%, 80%, 85, 90%, or 95%; and the ocular production of PPARγ and pro-resolving lipid mediators 5-HETE, 15-HETE and 17-OHDHA is increased by about 5%, 10%, 15%, 20%, 25%, 30%, 35%, 40%, 45%, 50%, 55%, 60%, 65%, 70%, 75%, 80%, 85, 90%, 95%, 100%, 500%, 1000%, or 10,000% relative to an ocular tissue that does not receive the composition.

Compositions comprising a COX2 Ser516 acetylating agent, alone or with a cPLA2 agonist may be used to decrease the expression of SMAD2/3 by ocular fibroblasts in a subject. In certain embodiments, the expression of SMAD2/3 by ocular fibroblasts is decreased relative to an ocular tissue that does not receive the composition. In an embodiment, the expression of SMAD2/3, MMPs and collagen by ocular fibroblasts, ocular prostaglandin production, inflammation, ocular fibrosis and/or scarring, ocular collagen contraction and/or remodeling and/or ocular fibroblast proliferation is decreased by about 5%, 10%, 15%, 20%, 25%, 30%, 35%, 40%, 45%, 50%, 55%, 60%, 65%, 70%, 75%, 80%, 85, 90%, or 95%; and the ocular production of PPARγ and pro-resolving lipid mediators 5-HETE, 15-HETE and 17-OHDHA is increased by about 5%, 10%, 15%, 20%, 25%, 30%, 35%, 40%, 45%, 50%, 55%, 60%, 65%, 70%, 75%, 80%, 85, 90%, 95%, 100%, 500%, 1000%, or 10,000% relative to an ocular tissue that does not receive the composition.

Compositions comprising a COX2 Ser516 acetylating agent, alone or with a cPLA2 agonist may be used to decrease the phosphorylation (activation) of SMAD2/3 by ocular fibroblasts in a subject. In certain embodiments, the phosphorylation (activation) of SMAD2/3 by ocular fibroblasts is decreased relative to an ocular tissue that does not receive the composition. In an embodiment, the phosphorylation of SMAD2/3; the expression of SMAD2/3, MMPs and collagen by ocular fibroblasts; ocular prostaglandin production, ocular inflammation, ocular fibrosis and/or scarring, ocular collagen contraction and/or remodeling and/or ocular fibroblast proliferation is decreased by about 5%, 10%, 15%, 20%, 25%, 30%, 35%, 40%, 45%, 50%, 55%, 60%, 65%, 70%, 75%, 80%, 85, 90%, or 95%; and the ocular production of PPARγ and pro-resolving lipid mediators 5-HETE, 15-HETE and 17-OHDHA is increased by about 5%, 10%, 15%, 20%, 25%, 30%, 35%, 40%, 45%, 50%, 55%, 60%, 65%, 70%, 75%, 80%, 85, 90%, 95%, 100%, 500%, 1000%, or 10,000% relative to an ocular tissue that does not receive the composition.

Compositions comprising a COX2 Ser516 acetylating agent, alone or with a cPLA2 agonist may be used to decrease the phosphorylation (activation) of SMAD2/3 by ocular fibroblasts in a subject. In certain embodiments, the metabolic activity of ocular fibroblasts is decreased relative to an ocular tissue that does not receive the composition. In an embodiment, the cellular metabolic activity, phosphorylation of SMAD2/3; the expression of SMAD2/3, MMPs and collagen by ocular fibroblasts; ocular prostaglandin production, ocular inflammation, ocular fibrosis and/or scarring, ocular collagen contraction and/or remodeling and/or ocular fibroblast proliferation is decreased by about 5%, 10%, 15%, 20%, 25%, 30%, 35%, 40%, 45%, 50%, 55%, 60%, 65%, 70%, 75%, 80%, 85, 90%, or 95%; and the ocular production of PPARγ and pro-resolving lipid mediators 5-HETE, 15-HETE and 17-OHDHA is increased by about 5%, 10%, 15%, 20%, 25%, 30%, 35%, 40%, 45%, 50%, 55%, 60%, 65%, 70%, 75%, 80%, 85, 90%, 95%, 100%, 500%, 1000%, or 10,000% relative to an ocular tissue that does not receive the composition.

### Kits

The present disclosure contemplates kits for carrying out the methods disclosed herein. Such kits comprise two or more components required for treatment of ocular tissue as provided herein. Components of the kit include, but are not limited to, a COX2 Ser516 acetylating agent, alone or with a cPLA2 agonist, and one or more of compounds, reagents, containers, equipment, and instructions for using the kit. Accordingly, the methods described herein may be performed by utilizing pre-packaged kits provided herein. In one embodiment, the kit comprises one or more compositions and instructions. In some embodiments, the instructions comprise one or more protocols for preparing and/or using the composition(s) in the method provided herein. In some embodiments, the kit comprises one or more reagents for performing a functional assay (to determine one or more indicators of ocular tissue function), or a molecular assay (to determine one or more molecular features of the ocular tissue) and instructions comprising one or more protocols for performing such assays, such as, for example, instructions for comparison to one or more standards. In some embodiments, the kit comprises one or more standards (e.g., standard comprising a biological sample, or representative transcript expression data).

While not wishing to be bound or limited by any theories, the inventors show herein that by increasing the activity of cPLA2 over baseline with the use of a cPLA2 agonist in the presence of COX2 Ser516 acetylating molecules, higher levels of arachadonic acid (AA), docosahexaenoic acid (DHA) and (eicosapentaenoic acid (EPA) are available for oxygenation by acetyl-COX2 and the endogenous LOX-5/15 enzymes to produce acetyl-COX2 products as well as endogenous lipoxygenase (LOX) products—all while inhibiting the derivation of prostaglandins (PGs) from AA through COX2 Ser516 acetylation. In this manner, PG synthesis is down-regulated, and the upstream products of an overactive PLA2 enzyme (AA, EPA and DHA; due to agonist in the composition) have a higher likelihood of being acted on by either the cell's own LOX-5/15 enzymes or acetyl-COX2 to generate the pro-resolving lipid mediators 5-HETE, 15-HETE and 17-OHDHA. The desirable actions of these pro-resolving lipid mediators are thus gained significantly earlier and in greater quantities than if COX2 is not acetylated or when COX2 is acetylated and no cPLA2 agonist is used.

While not wishing to be bound or limited by any theories, the acetylation of COX2 may modify the cellular lipid biosynthetic machinery such that the biosynthesis system, overall, greatly prefers the production of lipid mediators derived from omega 3 and omega 6 substrates that promote the resolution of inflammatory symptoms. The combination of COX2 Ser516 acetylation by an acetylating agent such as ASA and APHS with the upregulation of PLA2 (with an agonist) provides an unexpected means of increasing the bioavailability of upstream substrates generated by PLA2. These properties comprise the medically desirable effect of promoting the active resolution of inflammation, as opposed to the current standard interventions that rely on blocking the production of pro-inflammatory mediators.

Inflammation and its resolution are an endogenously controlled biological algorithm coding the intentional deviation and subsequent return to homeostasis that is required to overcome infection or trauma [56, 57]. The magnitude of tissue damage (fibrosis/scarring) that results from inflammatory insult is directly proportional to the intensity and even more so to the duration of the inflammatory reaction the body levies in response to that insult. In the eye, it is especially critical to ensure inflammatory responses are kept to a minimum—as the anatomy on which healthy vision is dependent can easily be disrupted by inflammation and fibrosis induced changes to tissue architecture, often with permanent consequences. The compositions and methods disclosed herein may promote active resolution of inflammation, as opposed to the current status quo anti-inflammatory drugs' aim of blocking or tempering the initial inflammatory deviation from homeostasis.

Amplifying the overall end products of the cPLA2 biosynthetic pathway would normally be undesirable, as it would ultimately amplify the production of PGs as well. The excess PGs produced would incite and/or potentiate inflammatory signalling, resulting in continued activation of immune cells and fibroblasts. The continued activity of these cells is medically unwanted and results in fibroblast proliferation, collagen contraction and myofibroblast differentiation. All are unwanted phenomena in the eye both perioperatively as well as before, during or after any inflammatory insult. This is why using a cPLA2 agonist, alone, in these situations would seem counter-intuitive. However, while not wishing to be bound by any theories, when administered in conjunction with a COX2 Ser516 acetylating agent, the cPLA2 agonist may result in acetyl-COX2 being exposed to greater relative concentrations of its substrates, and it produces from them the pro-resolving products 5-HETE, 15-HETE and 17-OHDHA instead of PGs. Thus, under these specific conditions, inflammatory, scarring and fibroproliferative pathology can be mitigated.

### Examples

### Example 1: Materials and Methods

### Isolation and Culture of HTCFs

Primary human Tenon's capsule fibroblast (HTCF) lines were derived from 0.2-0.4 cm surgically resected segments of Tenon's capsule using standard protocols [68]. The tissue specimens were extracted from male and female glaucoma patients undergoing primary trabeculectomy in London, Canada. Consent was acquired under the ethics review board of Western University, London, Canada. Ophthalmic surgeons removed segments of Tenon's capsule and placed them into primary culture growth media containing Dulbecco modified Eagle's minimal essential medium (DMEM), 10% Fetal Bovine Serum (FBS), 1% penicillin/streptomycin (P/S), and 1% amphotericin. After, specimens were placed in fibronectin coated 6-well culture plates, submerged in primary culture media, and subcultured upon confluency for use.

### Collagen Contraction Assay I

The delayed release fibroblast populated collagen lattice model was used to assess inhibition of gel contraction [58] by ASA and gentamicin. HTCFs were seeded at a density of 2.5×10⁵ cells/mL within an extracellular matrix (ECM) mixture containing 400 ul of rat tail-derived type I collagen (1.8 mg/ml), 80 ul of neutralizing solution (equal parts Waymouth's media (Gibco, CAT. NO: 11220035) and 0.275M NaOH) and 20 ul of HTCF conditioned media (concentrated to 25× to obtain a 1× final concentration within the 500 ul matrix solution). Cell free collagen lattices were prepared identically, however to isolate the effects of HTCFs alone on collagen, constructs without HTCFs were cast as a negative control. The cell-collagen solution was pipetted gently to ensure homogenous distribution of HTCFs while avoiding the production of air bubbles, then 500 ul were pipetted into each well of a 24-well tray. Collagen constructs were allowed to polymerize at 37 C for 45 minutes before adding low serum culture media containing DMEM, 2% Fetal Bovine Serum (FBS), 1% penicillin/streptomycin (P/S).

Tension was allowed to build up within the tethered matrix at 37 C and 5% CO2 for 72 hours before a sterile spatula was used to detach each collagen construct from the edges of the culture well. Plates were then immediately scanned on a flat bed laser scanner and then once per hour for the first 12 hours, then daily for a total of 4 days. The surface area of each collagen construct was measured using ImageJ (NIH) and standardized against the baseline surface area measurement to express changes in area as a percentage of original surface area.

### Collagen Contraction Assay I—Treatment Protocol

Vehicle control treatment solution comprised of DMEM with 2% FBS and 1% penicillin/streptomycin. The gentamicin treatment solution contained DMEM with 2% FBS, 1% penicillin/streptomycin and 1000 μg/ml gentamicin (w/vol) (Sigma-Aldrich, CAT. NO: G1397). The ASA treatment solution contained DMEM with 2% FBS, 1% penicillin/streptomycin and 2000 μg/ml ASA (w/vol) (Sigma-Aldrich, CAT. NO: A5376). The gentamicin-ASA combination treatment solution (Gent+ASA) contained DMEM with 2% FBS, 1% penicillin/streptomycin, 1000 μg/ml gentamicin (w/vol.) and 2000 μg/ml ASA (w/vo).

Culture media was removed from collagen constructs twice daily and replaced with one of the four treatment solutions for a duration of 15 minutes. Subsequently, the collagen constructs were washed with phosphate buffered saline (PBS, Gibco, CAT. NO: LS10010023) to remove remaining treatment solution and fresh culture media was replaced. This schedule continued from time of collagen construct casting for 4 days at which point the experiment concluded.

### ECM Remodelling

After seven days of culture, collagen constructs were fixed in 4% paraformaldehyde overnight. After fixation, BATs were dehydrated in ethanol, embedded in paraffin blocks, sectioned (5 μM) and mounted on glass microscope slides using standard methods. Sections were deparaffinized and hydrated using standard protocols. Sections were then stained with picrosirius red. Briefly, a solution of 0.1% Sirius red in saturated picric acid was applied for 60 min, followed by 2×0.5% acetic acid washes. Collagen birefringence, used to determine collagen fibrillar hue [59], was assessed by circularly polarized light microscopy of picrosirius red stained sections. Images were taken with an Abrio quantitative birefringence imaging system (Hinds Instruments) mounted on an Olympus BX-51 microscope. Specifically, a constant light intensity, a 45° angle to the polarizing filter and the same analyzer were used to facilitate comparisons between each sample.

When viewed under polarized light, the color of the collagen fibers stained with picrosirius red depends upon fiber thickness, spatial orientation and packing density; with the color changing from blue to yellow to orange to red as fiber maturity, thickness and density increase [60, 61]. This method has been used previously in rabbit experimental filtration surgery to examine subconjunctival fibrosis, and blue (green)/yellow staining was associated with improved bleb function [62]. Using ImageJ, this property was leveraged to determine the relative proportions of different color fibers within the stained collagen constructs. This quantitative method has been previously described [63], and is employed within the current experiment to assess changes in collagen architecture due to the presence of HTCFs. In short, relative color content of the images is obtained by separating the digital images into their hue, saturation and value components. The hue component contains information on the color of each pixel within the image. Every pixel can have one of 256 possible colors. To identify the relative proportions of red, orange, yellow and blue pixels within a given image, a propriety script was written and run using the following hue definitions within ImageJ: red 2-9 and 230-256, orange 10-38, yellow 39-51 and green 52-128. The number of pixels within each hue range is calculated and expressed as a proportion of the total number of pixels.

### Fluorescence Microscopy

Cellular proliferation was assessed through immunohistochemistry. Briefly, deparaffinized and hydrated sections were permeabilized for 30 minutes with 1% Triton X-100 in PBS. After blocking of nonspecific sites with 1% BSA in PBS, sections were incubated for 40 minutes with Alexa Fluor 568-conjugated primary antibody against Ki-67 (Abcam, CAT. NO: ab197234) a marker of active cellular proliferation [64]. Slides were then stained with 4,6-diamidino-2-phenylindole for 10 minutes and imaged with a laser-scanning confocal microscope (A1R HD Nikon Instruments Inc., Tokyo, Japan). Cells were counted by nuclei using ImageJ, and the proportion expressing Ki-67 was taken as an estimate of relative cellular proliferation between treatment groups. Ten random frames were taken per tissue section, with five tissue sections imaged per patient cell line and treatment group.

Expression of the contractile protein alpha smooth muscle actin (αSMA), the myofibroblastic phenotype marker [65], by HTCFs within collagen constructs was assessed through immunohistochemistry. Deparaffinized and hydrated sections were permeabilized for 30 minutes with 1% Triton X-100 in PBS. After blocking of nonspecific sites with 1% BSA in PBS, sections were incubated for 40 minutes with Alexa Fluor 568-conjugated primary antibody against αSMA (Abcam, CAT. NO: ab202295). Finally, slides were stained with 4, 6-diamidino-2-phenylindole for 10 minutes. For each tissue section, the area positive for αSMA staining was measured in ImageJ and then divided by the number of nuclei counted within that same frame, this number was then compared between treatment groups. Ten random frames were taken per tissue section, with five tissue sections imaged per patient cell line and treatment group.

Collagen constructs were cast with equal cell density, therefore there should be equal variance in cell density between treatment groups after the experimental incubation period. This was assessed through fluorescent microscopy. Briefly, deparaffinized and hydrated sections were permeabilized for 30 minutes with 1% Triton X-100 (Sigma-Aldrich) in PBS (Sigma-Aldrich). Slides were then stained with 4,6-diamidino-2-phenylindole (DAPI, Sigma-Aldrich) for 10 minutes and imaged with a laser-scanning confocal microscope (A1R HD Nikon Instruments Inc., Tokyo, Japan). Relative cell density was determined by cell (nucleus) count standardized to area of collagen autofluorescence (in pixels) within each section and measured using ImageJ. Ten random frames were taken with the 40× objective per tissue section, with three tissue sections imaged per patient cell line and treatment group. The laser intensity settings were kept consistent between slides to facilitate consistent comparison between replicates.

### Lipid Mediator Secretion Assay I

The aim of this experiment was to assess the relative secretion of COX2 vs. acetylated-COX2 products from HTCFs after an inflammatory stimulus followed by exposure to ASA with and without an extremely potent PLA2 agonist—melittin. To this end, multiple primary HTCF cell lines were cultured in DMEM w/10% FBS in 6-well culture plates, incubated at 37 C and 5% CO2, until 90% confluent. The culture media was removed, wells washed with PBS, and then fresh DMEM w/0% FBS was added. After 24 hrs of serum starvation, fresh DMEM w/0% FBS was added, this time containing a physiologically relevant inflammation/wound healing induction mixture (1 ng/ml each of: IL-1b, TNFa, IFNγ, TGFb). After 12 hrs incubation, the induction media was removed, the wells were washed with PBS and fresh DMEM w/0% FBS was added containing one of three experimental treatments for an incubation period of 24 hrs: 1) vehicle control (DMEM w/0% FBS), and 2) ASA 200 (DMEM w/0% FBS and 200 μg/ml ASA), and 3) ASA/Mel. (DMEM w/0% FBS, 200 μg/ml ASA and 10 μg/ml of melittin, a potent PLA2 agonist). At 6, 12 and 24 hrs after addition of the experimental treatments, 250 ul of supernatant was removed from each well, replaced with 250 ul of fresh DMEM w/0% FBS, and stored at −80° C. for subsequent LC-MS/MS analysis [66]. After 24 hrs, the wells were washed with ice cold PBS three times and total cellular protein from the HTCF monolayer was collected for quantification and subsequent western blot analyses. The absolute quantity of protein within each culture well's HTCF monolayer was used as a means to standardize the relative secretion of lipid mediators by HTCFs into the culture well's supernatant.

### Lipid Mediator Secretion Assay II

The aim of this experiment was to assess the relative secretion of COX2 vs. acetylated-COX2 products from HTCFs after an inflammatory stimulus followed by exposure to ASA or APHS with and without the less potent PLA2 agonist—gentamicin. To this end, as before, multiple primary HTCF cell lines were cultured in DMEM w/10% FBS in 6-well culture plates, incubated at 37° C. and 5% CO2, until 90% confluent. The culture media was removed, wells washed with PBS, and then fresh DMEM w/0% FBS was added. After 24 hrs of serum starvation, fresh DMEM w/0% FBS was added, this time containing a physiologically relevant inflammation/wound healing induction mixture (1 ng/ml each of: IL-1b, TNFa, IFNγ, TGFb). After 12 hrs incubation, the induction media was removed, the wells were washed with PBS and fresh DMEM w/0% FBS was added containing one of ten experimental treatments for an incubation period of 48 hrs: 1) non-induced control (NIC: received DMEM w/0% FBS instead of the induction media), and 2) CytoM1+TGFB1 (DMEM w/0% FBS and 1 ng/ml each of: IL-1b, TNFa, IFNγ, TGFb), and 3) CytoM1/TGFB1/Gent250 (DMEM w/0% FBS, 1 ng/ml each of TGFB1, TGFB2, VEGF, CCN2 and 250 μg/ml gentamicin), and 4) CytoM1/TGFB1/Gent500 (DMEM w/0% FBS, 1 ng/ml each of TGFB1, TGFB2, VEGF, CCN2 and 500 μg/ml gentamicin), and 5) CytoM1/TGFB1/ASA500 (DMEM w/0% FBS, 1 ng/ml each of TGFB1, TGFB2, VEGF, CCN2, and 500 μg/ml ASA), and 6) CytoM1/TGFB1/ASA500/Gent250 (DMEM w/0% FBS, 1 ng/ml each of TGFB1, TGFB2, VEGF, CCN2, 500 μg/ml ASA and 250 μg/ml gentamicin), and 7) CytoM1/TGFB1/ASA1000 (DMEM w/0% FBS, 1 ng/ml each of TGFB1, TGFB2, VEGF, CCN2 and 1000 μg/ml ASA), and 8) CytoM1/TGFB1/ASA1000/Gent500 (DMEM w/0% FBS, 1 ng/ml each of TGFB1, TGFB2, VEGF, CCN2, 1000 μg/ml ASA, and 500 μg/ml gentamicin), and 9) CytoM1/TGFB1/APHS12 (DMEM w/0% FBS, 1 ng/ml each of TGFB1, TGFB2, VEGF, CCN2 and 12 μg/ml o-(acetoxyphenyl)hept-2-ynyl sulfide (APHS), and 10) CytoM1/TGFB1/APHS12/Gent250 (DMEM w/0% FBS, 1 ng/ml each of TGFB1, TGFB2, VEGF, CCN2, 12 μg/ml o-(acetoxyphenyl)hept-2-ynyl sulfide (APHS) and 250 μg/ml gentamicin). At 6, 12, 24 and 48 hrs after addition of the experimental treatments, 250 ul of supernatant was removed from each well, replaced with 250 ul of fresh DMEM w/0% FBS, and stored at −80° C. for subsequent LC-MS/MS analysis. After 48 hrs, the wells were washed with ice cold PBS three times and total cellular protein from the HTCF monolayer was collected for quantification and subsequent western blot analyses. The absolute quantity of protein within each culture well's HTCF monolayer was used as a means to standardize the relative secretion of lipid mediators by HTCFs into the culture well's supernatant.

### Cellular Metabolic Activity Assays

The effects of COX2 acetylation on HTCF and induced-myofibroblast metabolic activity was assessed using a 3-[4,5-dimethylthiazol-2-yl]-2,5-diphenyltetrazolium bromide (MTT, Sigma-Aldrich) assay. Cells were cultured in 24-well culture plates in DMEM w/10% FBS until 80-90% confluent, then they were starved of serum for 24 hrs. After, they were treated with one of the following experimental treatments: 1) vehicle control (DMEM w/0% FBS), 2) TGFb1-induced positive control (DMEM w/0% FBS and 1 ng/ml TGFb1), 3) ASA (DMEM w/0% FBS and 100 to 3200 μg/ml ASA), 4) TGFb1+ASA (DMEM w/0% FBS, 1 ng/ml TGFb1 and 100 to 3200 μg/ml ASA), 5) APHS (DMEM w/0% FBS and 4 to 16 μg/ml APHS), and 6) TGFB1+APHS (DMEM w/0% FBS, 1 ng/ml TGFb1 and 100 to 3200 μg/ml ASA) for 48 hrs. After, cells were incubated in DMEM w/0% FBS and 500 μg/ml MTT for 4 hrs at 37 C and 5% CO2. Media was then removed, cells washed and then lysed in DMSO to solubilize the formazan crystals that have formed by the reduction of MTT within metabolically active cells. Relative formazan concentration was compared between treatment groups by measuring the optical density at 570 nm.

### Total Protein Isolation, Quantification and Western Blot Assay

Cells were lysed in lysis buffer (PhosphoSafe Extraction Reagent, Novagen) containing a protease inhibitor cocktail (P2714, Sigma-Aldrich). Crude protein lysate was quantified using a Pierce BCA protein assay kit (ThermoFisher, Mississauga, ON) and 10 ug from each sample was resolved using a Novex WedgeWell 4-20% tris-glycine gel (Invitrogen). Using an iBlot Gel Transfer Device (IB1001, Invitrogen), the separated protein was transferred to a nitrocellulose membrane (1B301001, iBlot Transfer Stack, Invitrogen) which was then blocked with 5% (w/v) bovine serum albumin (Sigma-Aldrich) in Tris buffered saline (TBST) for 1 hour at room temperature. The membranes were incubated overnight at 4° C. with primary antibody diluted in TBST containing 5% BSA (w/vol). Primary antibodies used were as follows: collagen 1 (ab138492, Abcam), αSMA (ab5694, Abcam), MMP9 (ab38898, Abcam), PPARγ (sc-7273, SantaCruz Biotechnology), SMAD2/3 (ab63672, Abcam), pSMAD2/3 (ab63399, Abcam) and GAPDH (sc-47724, Santa Cruz Biotechnology, Inc.). After incubation with primary antibodies, the blots were washed and hybridized with 1:3000 (v/v) dilutions of the appropriate goat anti-rabbit or anti-mouse IgG conjugated with horseradish peroxidase (U.S. Pat. Nos. 1,706,515 and 1,721,011, respectively, both from Bio-Rad Laboratories, Inc.). Visualization was accomplished by applying WesternBright Quantum chemiluminescent reagent (Advansta, Inc.), with GAPDH used as a protein loading control. Imaging and relative densiometric quantification was accomplished using a ChemiDoc MP System (Bio-Rad Laboratories, Inc.) connected to Image Lab (Version 6, Bio-Rad Laboratories, Inc.).

### Relative Quantification of Secreted Lipid Mediators and Polyunsaturated Fatty Acids

Relative quantification of secreted lipid mediators was achieved using liquid chromatography tandem mass spectrometry combined with the addition of a known quantity of deuterated versions of the lipid mediators to each sample as internal standards (Item Numbers: #10007737, #320110, #314010, #11182, #315210, #319030, #390030, #10006410, #10006199, #334230, and #10008040, Cayman Chemical). This is a well-established method to determine the relative activity of cyclooxygenase, acetyl-COX2 and lipoxygenase biosynthetic pathways [66, 67]. In brief, the ratios of the integrated areas of the chromatographic peaks corresponding to each analyte and the integrated areas of the peaks corresponding to each analyte's internal standard (with known absolute quantity) are used to determine the relative quantity of each analyte in a given sample. After supernatant sample collection, 250 ul of methanol containing 100 μg of each deuterated internal standard was added. Samples were then vigorously vortexed and centrifuged at 10,000 g for 10 min before loading onto solid-phase extraction cartridges (Strata-X 33 um Polymeric Reversed Phase, 10 mg, Phenomenex 8B-S100-AAK), that were previously activated with 2 ml methanol and rinsed with 2 ml water. Samples were diluted upon loading so that the final concentration of methanol was between 10 and 15% of total volume. After washing with 5 ml water, extracts were eluted with 1 ml methanol. Solvent was then evaporated under vacuum in a SpeedVac centrifuge, and the extract was resuspended in 100 ul acetonitrile/water 60:40 (v/v). An aliquot of 20 ul was injected into the LC-MS/MS system for analysis. A Sciex ExionLC Integrated System was used with a 0.3 ml/min flow rate, initial mobile phase of 10% water/0.1% formic acid followed by 100% acetonitrile/0.1% formic acid on a Kinetex 2.6 um C18 100 Å 100×2.1 mm, Phenomax column (OOD-4462-AN). A Sciex Triple Quad 6500+ mass spectrometer with multiple reaction monitoring was used in negative ion mode. The chromatographic profile of the ion count for each m/z transition was monitored, and the area under the peaks (ion intensity vs elution time) was integrated using commercial software (MultiQuant, Sciex).

### Cytotoxicity Assays

To assess the relative cellular viability between different experimental conditions, an in situ fluorescence-based LIVE/DEAD assay was used. This is a well reported method for estimating the cytotoxicity of an intervention. In brief, two florescent dyes, fluorescein diacetate and propidium iodide, are added to the culture media surrounding the collagen constructs. Fluorescein diacetate is converted into a blue fluorescent molecule by esterases within living cells. Propidium iodide (red) cannot pass through a viable cell's membrane, however it can penetrate disordered areas of dead cell membranes and then intercalates with the nuclear DNA. After a 5 min incubation with the staining solution, collagen constructs were washed with PBS and immediately imaged on a laser-scanning confocal microscope (A1R HD Nikon Instruments Inc., Tokyo, Japan). Ten random frames were taken with the 20× objective per tissue section, with three tissue sections imaged per patient cell line and treatment group. The laser intensity settings were kept consistent between samples to facilitate consistent comparison between replicates.

Using ImageJ, blue signals were counted within every frame and counted as living cells. The same was done for red signals, and these were counted as dead cells. The ratio of living (blue) to total (blue+red) cells was recorded.

### Collagen Contraction Assay II

The delayed release fibroblast populated collagen lattice model was used to assess the dose dependent inhibition of gel contraction by ASA and the augmentation of this effect in a dose dependent manner by gentamicin. HTCFs were seeded at a density of 2.5×105 cells/mL within an extracellular matrix (ECM) mixture containing 400 ul of rat tail-derived type I collagen (1.8 mg/ml), 80 ul of neutralizing solution (equal parts Waymouth's media (Gibco, CAT. NO: 11220035) and 0.275M NaOH) and 20 ul of HTCF conditioned media (concentrated to 25× to obtain a 1× final concentration within the 500 ul matrix solution). The cell-collagen solution was pipetted gently to ensure homogenous distribution of HTCFs while avoiding the production of air bubbles, then 500 ul were pipetted into each well of a 24-well tray. Collagen constructs were allowed to polymerize at 37 C for 45 minutes before adding low serum culture media containing DMEM, 2% Fetal Bovine Serum (FBS), 1% penicillin/streptomycin (P/S).

Tension was allowed to build up within the tethered matrix at 37 C and 5% CO2 for 72 hours before a sterile spatula was used to detach each collagen construct from the edges of the culture well. Plates were then immediately scanned on a flat bed laser scanner once per 24 hours for a total of 4 days. The surface area of each collagen construct was measured using ImageJ (NIH) and standardized against the baseline surface area measurement to express changes in area as a percentage of original surface area.

### Collagen Contraction Assay II— Treatment Protocol

The gentamicin concentrations assessed were delivered in a solution contained DMEM with 2% FBS, 1% penicillin/streptomycin and either 100, 250, 500, 750 or 100 μg/ml gentamicin (Sigma-Aldrich, CAT. NO: G1397). The ASA concentrations assessed were delivered in a solution of DMEM with 2% FBS, 1% penicillin/streptomycin and either 500, 1000 or 1500 μg/ml ASA (Sigma-Aldrich, CAT. NO: A5376). Ultimately, the 24 treatment groups were as follows: 1) vehicle control (DMEM w/2% FBS), 2) 100G (DMEM w/2% FBS and 100 μg/ml gent, 3) 250G (DMEM w/2% FBS and 250 μg/ml gent), 4) 500G (DMEM w/2% FBS and 500 μg/ml gent), 5) 750G (DMEM w/2% FBS and 750 μg/ml gent), 6) 1000G (DMEM w/2% FBS and 1000 μg/ml gent), 7) 500ASA (DMEM w/2% FBS and 500 μg/ml ASA), 8) 1000ASA (DMEM w/2% FBS and 1000 μg/ml ASA), 9) 1500ASA (DMEM w/2% FBS and 1500 μg/ml ASA), 10) 500ASA+100G (DMEM w/2% FBS, 500 μg/ml ASA and 100 μg/ml gentamicin), 11) 500ASA+250G (DMEM w/2% FBS, 500 μg/ml ASA and 250 μg/ml gentamicin), 12) 500ASA+500G (DMEM w/2% FBS, 500 μg/ml ASA and 500 μg/ml gentamicin), 13) 500ASA+750G (DMEM w/2% FBS, 500 μg/ml ASA and 750 μg/ml gentamicin), 14) 500ASA+1000G (DMEM w/2% FBS, 500 μg/ml ASA and 1000 μg/ml gentamicin), 15) 1000ASA+100G (DMEM w/2% FBS, 1000 μg/ml ASA and 100 μg/ml gentamicin), 16) 1000ASA+250G (DMEM w/2% FBS, 1000 μg/ml ASA and 250 μg/ml gentamicin), 17) 1000ASA+500G (DMEM w/2% FBS, 1000 μg/ml ASA and 500 μg/ml gentamicin), 18) 1000ASA+750G (DMEM w/2% FBS, 1000 μg/ml ASA and 750 μg/ml gentamicin), 19) 1000ASA+1000G (DMEM w/2% FBS, 1000 μg/ml ASA and 1000 μg/ml gentamicin), 20) 1500ASA+100G (DMEM w/2% FBS, 1500 μg/ml ASA and 100 μg/ml gentamicin), 21) 1500ASA+250G (DMEM w/2% FBS, 1500 μg/ml ASA and 250 μg/ml gentamicin), 22) 1500ASA+500G (DMEM w/2% FBS, 1500 μg/ml ASA and 500 μg/ml gentamicin), 23) 1500ASA+750G (DMEM w/2% FBS, 1500 μg/ml ASA and 750 μg/ml gentamicin), 24) 1500ASA+1000G (DMEM w/2% FBS, 1500 μg/ml ASA and 1000 μg/ml gentamicin).

Culture media was removed from collagen constructs 30 minutes before detachment with the sterile spatula and replaced with experimental treatment media described above. After 30 minutes, collagen constructs were then detached from the sides of the culture wells and allowed to contract under experimental conditions for an observation period of four days.

### Collagen Contraction Assay III

TGFB1 is a major wound healing and scarring associated cytokine found at significantly elevated levels within the aqueous humor of glaucoma patients. A modified delayed release fibroblast populated collagen lattice model was used to assess inhibition of TGFB1-induced collagen contraction by ASA and gentamicin. HTCFs were seeded at a density of 2.5×105 cells/mL within an extracellular matrix (ECM) mixture containing 400 ul of rat tail-derived type I collagen (1.8 mg/ml), 80 ul of neutralizing solution (equal parts Waymouth's media (Gibco, CAT. NO: 11220035) and 0.275M NaOH) and 20 ul of HTCF conditioned media (concentrated to 25× to obtain a 1× final concentration within the 500 ul matrix solution). The cell-collagen solution was pipetted gently to ensure homogenous distribution of HTCFs while avoiding the production of air bubbles, then 500 ul were pipetted into each well of a 24-well tray. Collagen constructs were allowed to polymerize at 37 C for 45 minutes before adding low serum culture media containing DMEM, 2% Fetal Bovine Serum (FBS), 1% penicillin/streptomycin (P/S).

Tension was allowed to build up within the tethered matrix at 37 C and 5% CO2 for 72 hours before a sterile spatula was used to detach each collagen construct from the edges of the culture well. Plates were then immediately scanned on a flat bed laser scanner for a baseline area reading and then measured again regularly for a total of 4 days. In this modified assay, at 48 hrs of contraction, TGFB1 (2 ng/ml) was added to the culture media of all treatment groups. The surface area of each collagen construct was measured using ImageJ (NIH) and standardized against the baseline surface area measurement to express changes in area as a percentage of original surface area.

### Collagen Contraction Assay III— Treatment Protocol

Exposure to experimental treatment solutions occurred from 30 min before the detachment of collagen constructs from the edges of the culture wells, up until 48 hrs, when the wells were then washed with PBS and media replaced with the TGFB1 induction media for the remainder of the experiment (DMEM w/2% FBS and 2 ng/ml TGFB1). The experimental treatment solutions were as follows: 1) vehicle control (DMEM w/2% FBS), 2) pH control (DMEM w/2% FBS and hydrochloric acid added equimolar to 1500 μg/ml ASA so as to provide equivalent H⁺ to the buffered culture media), 3) 1000ASA (DMEM w/2% FBS and 1000 μg/ml ASA), 4) 1000ASA+333G (DMEM w/2% FBS, 1000 μg/ml ASA and 333 μg/ml gentamicin), 5) 1000ASA+500G (DMEM w/2% FBS, 1000 μg/ml ASA and 500 μg/ml gentamicin), 6) 1500ASA (DMEM w/2% FBS and 1500 μg/ml ASA), 7) 1500ASA+500G (DMEM w/2% FBS, 1500 μg/ml ASA and 500 μg/ml gentamicin), 8) 1500ASA+750G (DMEM w/2% FBS, 1500 μg/ml ASA and 750 μg/ml gentamicin.

### Collagen Contraction Assay IV

A modified delayed release fibroblast populated collagen lattice model was used to assess the effects of ASA and gentamicin on the inhibition of aqueous humor growth factor (AHGF) induced gel contraction—and compare the effect size to that of the current clinical gold standard, mitomycin C. The AHGFs mixture contained inflammatory and pro-scarring cytokines (IL-1b, TNFa, IFNγ, TGFB1, TGFB2, CCN2 and VEGF) known to be significantly upregulated within the aqueous humor of glaucoma patients. HTCFs were seeded at a density of 2.5×105 cells/mL within an extracellular matrix (ECM) mixture containing 400 ul of rat tail-derived type I collagen (1.8 mg/ml), 80 ul of neutralizing solution (equal parts Waymouth's media (Gibco, CAT. NO: 11220035) and 0.275M NaOH) and 20 ul of HTCF conditioned media (concentrated to 25× to obtain a 1× final concentration within the 500 ul matrix solution). The cell-collagen solution was pipetted gently to ensure homogenous distribution of HTCFs while avoiding the production of air bubbles, then 500 ul were pipetted into each well of a 24-well tray. Collagen constructs were allowed to polymerize at 37 C for 45 minutes before adding low serum culture media containing DMEM, 2% Fetal Bovine Serum (FBS), 1% penicillin/streptomycin (P/S).

Tension was allowed to build up within the tethered matrix at 37 C and 5% CO2 for 72 hours before a sterile spatula was used to detach each collagen construct from the edges of the culture well. Plates were then immediately scanned on a flat bed laser scanner and then periodically for a total of 4 days. The surface area of each collagen construct was measured using ImageJ (NIH) and standardized against the baseline surface area measurement to express changes in area as a percentage of original surface area.

### Collagen Contraction Assay IV—Treatment Protocol

In this modification of the assay, AHGFs are added to all culture media the collagen constructs are exposed to—from casting of the collagen constructs to the conclusion of contraction. The experimental treatment solutions—all also containing AHGFs—were added 30 minutes prior to detaching the collagen constructs from the culture wells and remained until experimental conclusion. Experimental treatments were as follows: 1) vehicle control (DMEM w/2% FBS), 2) aqueous humor growth factors (AHGFs: DMEM w/2% FBS and 2 ng/ml each of TGFB1, TGFB2, VEGF, CCN2), 3) AHGFs/Gent750 (DMEM w/2% FBS, 2 ng/ml each of TGFB1, TGFB2, VEGF, CCN2 and 750 μg/ml gentamicin), 4) AHGFs/ASA1500 (DMEM w/2% FBS, 2 ng/ml each of TGFB1, TGFB2, VEGF, CCN2 and 1500 μg/ml ASA), 5) AHGFs/ASA1500/Gent750 (DMEM w/2% FBS, 2 ng/ml each of TGFB1, TGFB2, VEGF, CCN2, 1500 μg/ml ASA and 750 μg/ml gentamicin), and 6) AHGFs/MMC (before detachment of these collagen scaffolds from culture wells, they were exposed to 0.2 mg/ml mitomycin C in PBS for 4 minutes prior to being subsequently washed twice with fresh PBS. DMEM w/2% FBS was added for the remainder of the experiment).

### Collagen Contraction Assay V

A modified delayed release fibroblast populated collagen lattice model was used to assess the effects of o-(acetoxyphenyl)hept-2-ynyl sulfide (APHS) and gentamicin on the inhibition of aqueous humor growth factor (AHGF) induced gel contraction, and to compare the effect size to that of a current clinical gold standard, mitomycin C. The AHGFs mixture contained inflammatory and pro-scarring cytokines (IL-1b, TNFa, IFNγ, TGFB1, TGFB2, CCN2 and VEGF) known to be significantly upregulated within the aqueous humor of glaucoma patients. HTCFs were seeded at a density of 2.5×105 cells/mL within an extracellular matrix (ECM) mixture containing 400 ul of rat tail-derived type I collagen (1.8 mg/ml), 80 ul of neutralizing solution (equal parts Waymouth's media (Gibco, CAT. NO: 11220035) and 0.275M NaOH) and 20 ul of HTCF conditioned media (concentrated to 25× to obtain a 1× final concentration within the 500 ul matrix solution). The cell-collagen solution was pipetted gently to ensure homogenous distribution of HTCFs while avoiding the production of air bubbles, then 500 ul were pipetted into each well of a 24-well tray. Collagen constructs were allowed to polymerize at 37 C for 45 minutes before adding low serum culture media containing DMEM, 2% Fetal Bovine Serum (FBS), 1% penicillin/streptomycin (P/S).

Tension was allowed to build up within the tethered matrix at 37 C and 5% CO2 for 72 hours before a sterile spatula was used to detach each collagen construct from the edges of the culture well. Plates were then immediately scanned on a flat bed laser scanner to record baseline area, and then measured periodically for a duration of 4 days. The surface area of each collagen construct was measured using ImageJ (NIH) and standardized against the baseline surface area measurement to express changes in area as a percentage of original surface area.

### Collagen Contraction Assay V—Treatment Protocol

In this modification of the assay, AHGFs are added to all culture media the collagen constructs are exposed to—from casting of the collagen constructs to the conclusion of contraction. The experimental treatment solutions—all also containing AHGFs—were added 30 minutes prior to detaching the collagen constructs from the culture wells and remained until experimental conclusion. Experimental treatments were as follows: 1) aqueous humor growth factors (AHGFs: DMEM w/2% FBS and 2 ng/ml each of TGFB1, TGFB2, VEGF, CCN2), 2) AHGFs/Gent750 (DMEM w2% FBS, 2 ng/ml each of TGFB1, TGFB2, VEGF, CCN2, and 750 μg/ml gentamicin), 3) AHGFs/APHS 12 (DMEM w/2% FBS, 2 ng/ml each of TGFB1, TGFB2, VEGF, CCN2 and 12 μg/ml o-(acetoxyphenyl)hept-2-ynyl sulfide (APHS)), 4) AHGFs/APHS12/Gent750 (DMEM w/2% FBS, 2 ng/ml each of TGFB1, TGFB2, VEGF, CCN2, 12 μg/ml o-(acetoxyphenyl)hept-2-ynyl sulfide (APHS) and 750 μg/ml gentamicin), 5) AHGFs/APHS 24 (DMEM w/2% FBS, 2 ng/ml each of TGFB1, TGFB2, VEGF, CCN2 and 24 μg/ml o-(acetoxyphenyl)hept-2-ynyl sulfide (APHS)), 6) AHGFs/APHS24/Gent750 (DMEM w/2% FBS, 2 ng/ml each of TGFB1, TGFB2, VEGF, CCN2, 24 μg/ml o-(acetoxyphenyl)hept-2-ynyl sulfide (APHS) and 750 μg/ml gentamicin), and 7) MMC/AHGFs (before detachment of these collagen scaffolds from culture wells, they were exposed to 0.2 mg/ml mitomycin C in PBS for 4 minutes prior to being subsequently washed twice with fresh PBS. DMEM w/2% FBS and 2 ng/ml each of TGFB1, TGFB2, VEGF, CCN2 was added for the remainder of the experiment).

### Cellular Response to Individual Exogenous ASA-Triggered Lipid Mediators

From the lipid mediator secretion assays, we see that exposure to ASA or APHS±a PLA2-a results in the generation of significant quantities of pro-resolving products 5-HETE, 11-HETE, 15-HETE and/or 17-OHDHA. The aim of this experiment was to assess the effects (both individually and in combination) of these lipid mediators at mitigating the induction of αSMA by aqueous humor growth factors. To this end, multiple primary HTCF cell lines were cultured in DMEM w/10% FBS in 6-well culture plates, incubated at 37 C and 5% CO2, until 80% confluent. The culture media was removed, wells washed with PBS, and then fresh DMEM w/0% FBS was added. After 24 hrs of serum starvation, one of 14 experimental treatments were applied for 48 hrs: 1) vehicle control (DMEM w/0% FBS), and 2) AHGFs (DMEM w/0% FBS and 2 ng/ml each of TGFB1, TGFB2, VEGF, CCN2), and 3-5) AHGFs/5-HETE (DMEM w/0% FBS, 2 ng/ml each of TGFB1, TGFB2, VEGF, CCN2, and 10, 100, or 1000 nM of 5-hydroxyeicosatetraenoic acid), and 6-8) AHGFs/11-HETE (DMEM w/0% FBS, 2 ng/ml each of TGFB1, TGFB2, VEGF, CCN2, and 10, 100, or 1000 nM of 11-hydroxyeicosatetraenoic acid), and 9-11) AHGFs/15-HETE (DMEM w/0% FBS, 2 ng/ml each of TGFB1, TGFB2, VEGF, CCN2, and 10, 100, or 1000 nM of 15-hydroxyeicosatetraenoic acid), and 12-14) AHGFs/17-OHDHA (DMEM w/0% FBS, 2 ng/ml each of TGFB1, TGFB2, VEGF, CCN2, and 10, 100, or 1000 nM of 17-hydroxy-docosahexaenoic acid). After 48 hrs exposure cellular protein lysate was collected and assessed by western blot with primary antibodies for αSMA and GAPDH, as described previously.

### Patient Replicates and Technical Replicates

Throughout the experimental reporting, “N” represents the number of different patient cell lines used for each experiment (patient/biological replicates) and “n” represents the number of times each experiment was repeated within each given patient cell line (technical replicates).

### Statistical Analysis

All data are presented as means±SD unless otherwise indicated. All experiments were performed, at a minimum, in technical triplicate for each primary HTCF cell line. Collagen contraction assay data and lipid mediator relative secretion assay data were subject to two-way repeated measures analysis of variance followed by the Tukey-Kramer multiple comparisons test, if necessary, with use of statistical and graphing software Prism 7 (Version 7.03, GraphPad Software Inc.). For collagen remodelling assay data, the relative area of collagen stained blue, yellow, orange or red, under different treatment conditions were assessed by one-way ANOVA with subsequent Dunnett's multiple comparisons test, with a single pooled variance if necessary. This same statistical method was used for cytotoxicity and western blot assay data. For florescence microscopy data, Student's t-test were used. For all tests, a P value of less than 0.05 was considered statistically significant.

### Example 2: Results

### Collagen Contraction Assay I

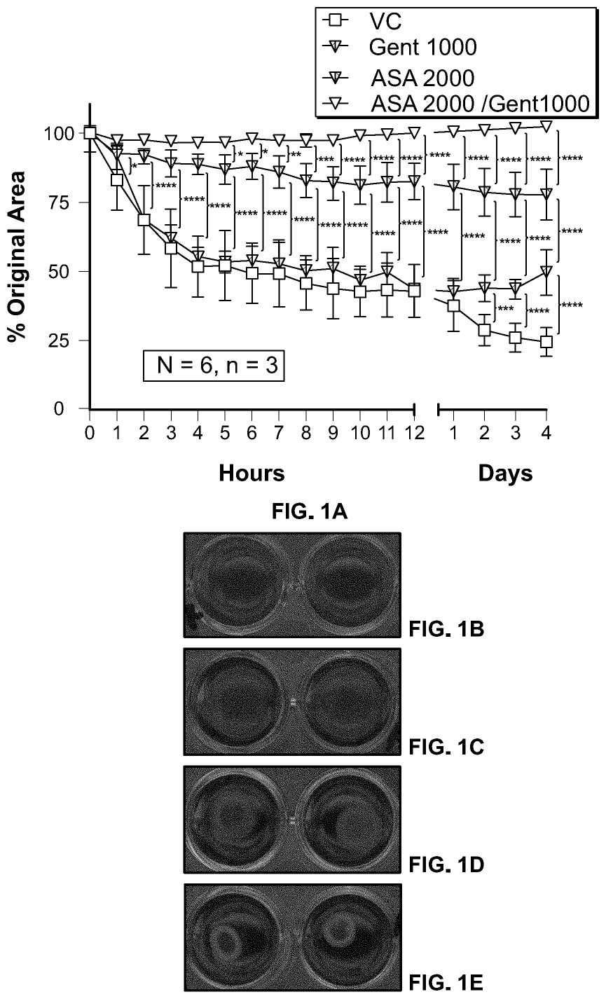

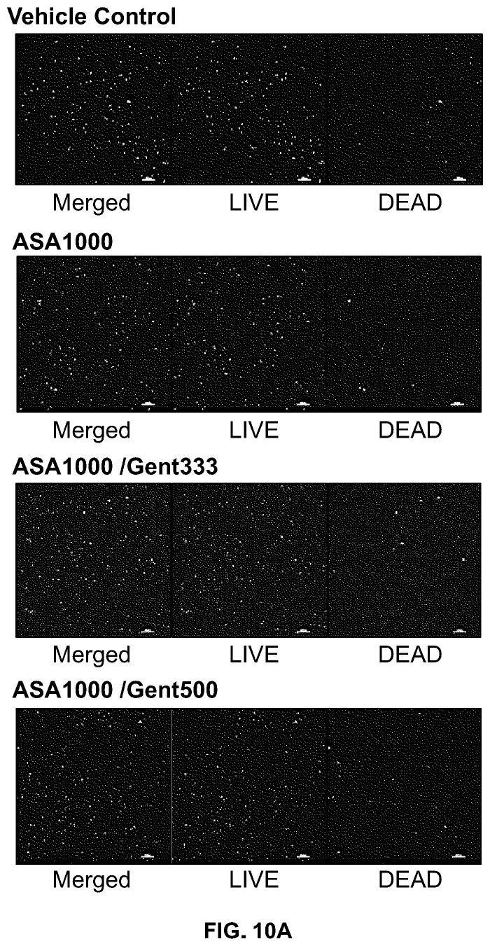

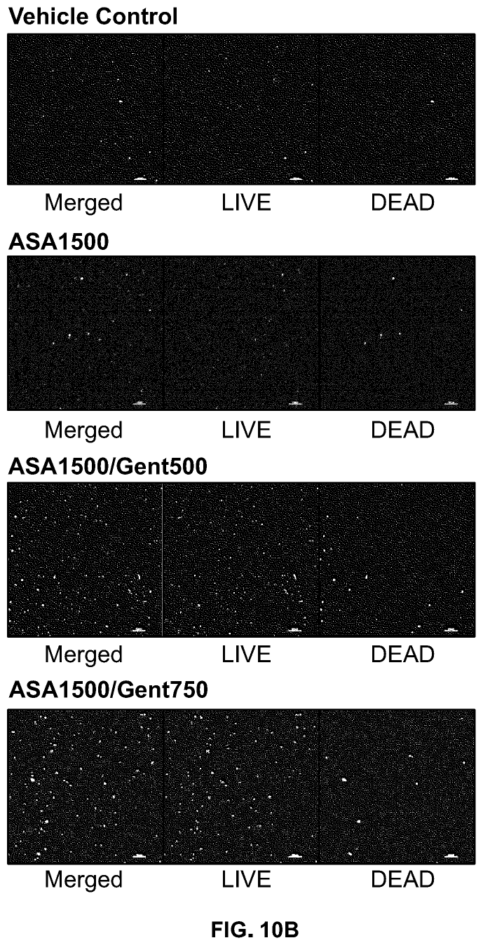

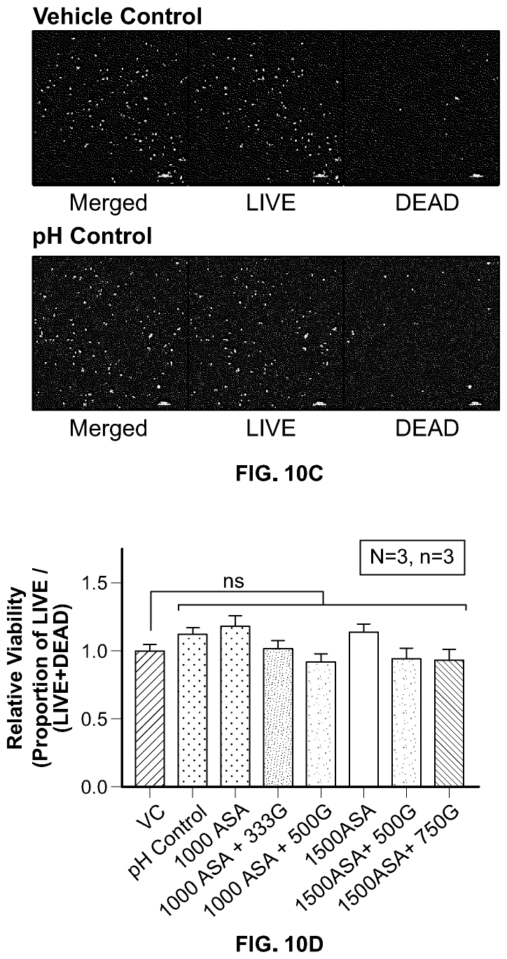

An in vitro assay utilizing HTCFs seeded within delayed-release collagen matrices was employed to measure the effects of drug exposures on HTCF mediated collagen contraction. This method has been used extensively in vitro to evaluate the potential antifibrotic activity of a drug (46, 47). Exposure to gentamicin treatment solution twice daily for 15 minutes did not affect collagen contraction until the second day of the four day assay (FIGS. 1A and 1D). ASA alone significantly inhibited contraction compared to vehicle control at all time points after the first hour of the experiment, however an approximately 20% contraction was evident by the end of the four-day period (FIGS. 1A and 10). Treatment with combination gentamicin and ASA significantly inhibited contraction at all time points. P values were not calculated as significance is adequately illustrated by the graphed means and 95% confidence intervals (FIGS. 1A and 1B). Gentamicin did not significantly affect contraction relative to VC until 48 hrs after the experiment began. ASA alone had immediate benefits (1 hr) over VC. The combination treatment had immediate benefits over VC at all time points. The combination of gentamicin and ASA was significantly better than ASA alone at all time points. These results are surprising, because if the effects of the two drugs were additive and independent from one another, the improvement of the combination over ASA alone would only be evident by 48 hrs of the experiment, as this is when the effects of gentamicin alone were observed. Instead, what was seen was an improvement over ASA at all time points, indicating that the effects of ASA are in fact potentiated by gentamicin.

### ECM Remodelling

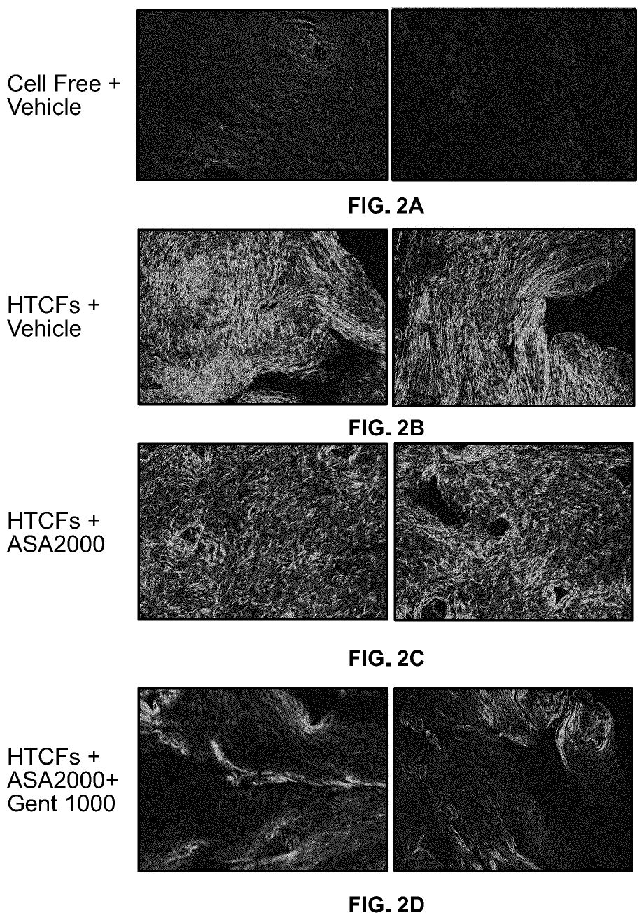

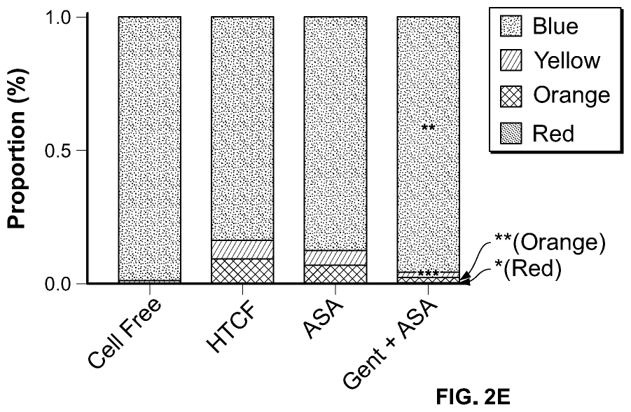

Picrosirius red staining was used in combination with circularly polarized light as a highly sensitive means to visualize collagen fibers as well as obtain information on fibrillar hue. The fibrillar hue was used to assess structural changes induced by HTCFs on the collagen matrix. As cell-mediated collagen fiber thickness and/or maturity increases, the fibrillar hue progresses from green to yellow to orange to red [60-63]. Empty collagen constructs, cast without HTCFs and incubated for seven days under experimental conditions stained predominantly blue, indicating the most immature collagen fibers and representing baseline collagen architecture (FIG. 2A). Constructs containing HTCFs revealed a predominance of densely packed, mature red/orange staining collagen fibers (FIG. 2B)—which are inferred to be cell mediated, structural changes in the architecture of the collagen matrix. Upon comparison to FIG. 2A, it becomes evident that HTCFs act to remodel the matrix over time, changing the pattern observed in cell-free scaffolds from a uniform blue to a pattern that include areas of red, orange and yellow staining—indicating regions of higher collagen remodelling. Constructs treated with ASA alone exhibited marked reduction in red, orange and yellow staining intensities (FIG. 2C), suggesting mitigation of the HTCFs collagen remodelling activity. Sections of constructs treated with the gentamicin/ASA combination (FIG. 2D) displayed even more significantly reduced areas of red/orange/yellow staining compared to HTCF containing constructs treated with vehicle treatment solutions, indicating less collagen remodelling had taken place over the incubation period and that gentamicin was potentiating the effects of ASA (FIG. 2E).

### Fluorescence Microscopy

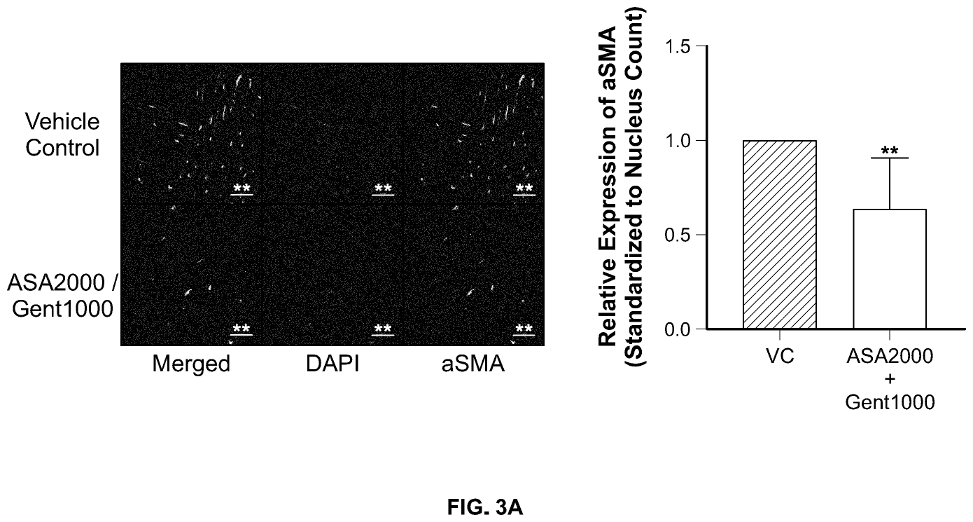

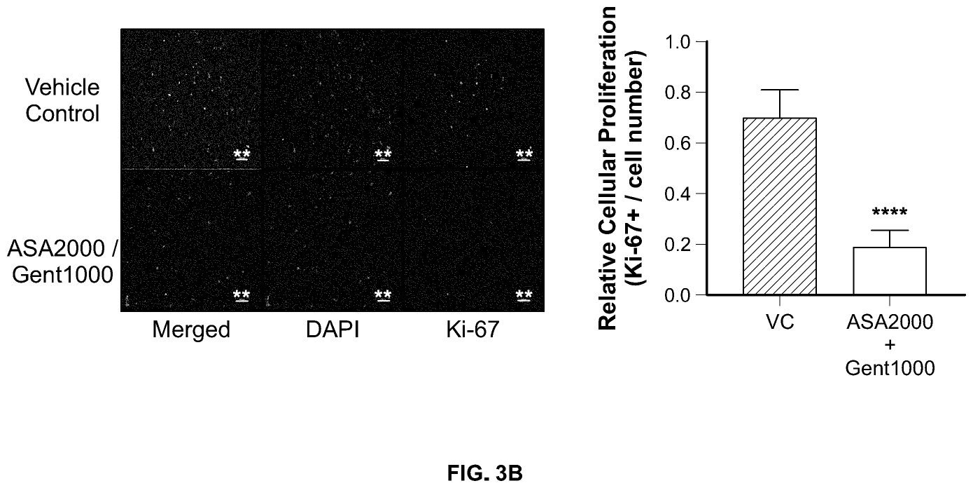

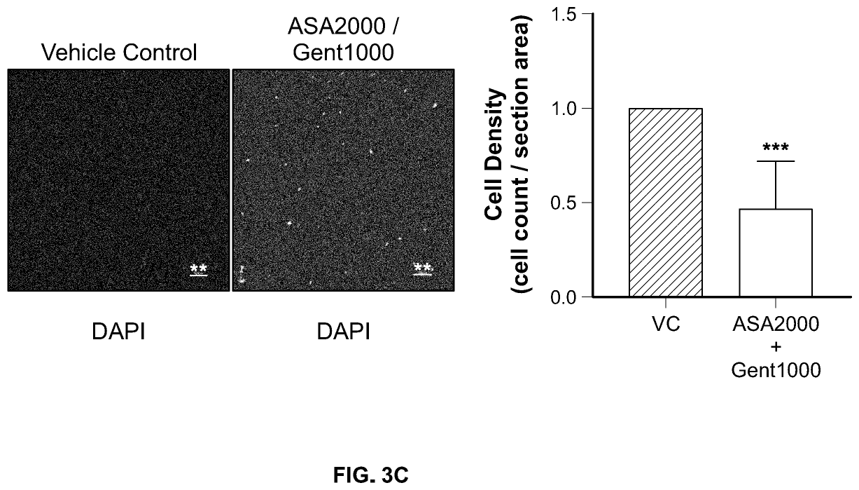

The contractile fibroblast phenotype, the myofibroblast, was identified through the protein marker alpha smooth muscle actin (αSMA). Elaboration of αSMA by HTCFs within the collagen constructs was assessed through immunohistochemistry. For each tissue section, the area staining positive for αSMA was normalized to the number of nuclei counted within that same frame, this number was then compared between treatment groups. Constructs incubated with combination gentamicin and ASA exhibited significantly reduced αSMA staining per nuclei compared to those treated with vehicle solution (FIG. 3A).

Ki-67 was used to assess relative differences in cellular proliferation between treatment groups. Constructs treated with combination gentamicin and ASA solutions exhibited significantly fewer cells staining positive for Ki-67 compared to constructs treated with the vehicle control solution (FIG. 3B).

The number of DAPI stained nuclei per unit area of collagen matrix visible within each micrograph were used to calculate the average cellular density within the 3D culture system after seven days of experimental conditions. Constructs treated with combination gentamicin and ASA exhibited significantly fewer cells (nuclei) per unit area of collagen matrix compared to constructs treated with the vehicle control solution (FIG. 3C). As the cellular density at the beginning of the experiment were equal between treatment groups, the observed effect indicates that treatment was likely to have inhibited cellular growth.

### Lipid Mediator Secretion Assay I

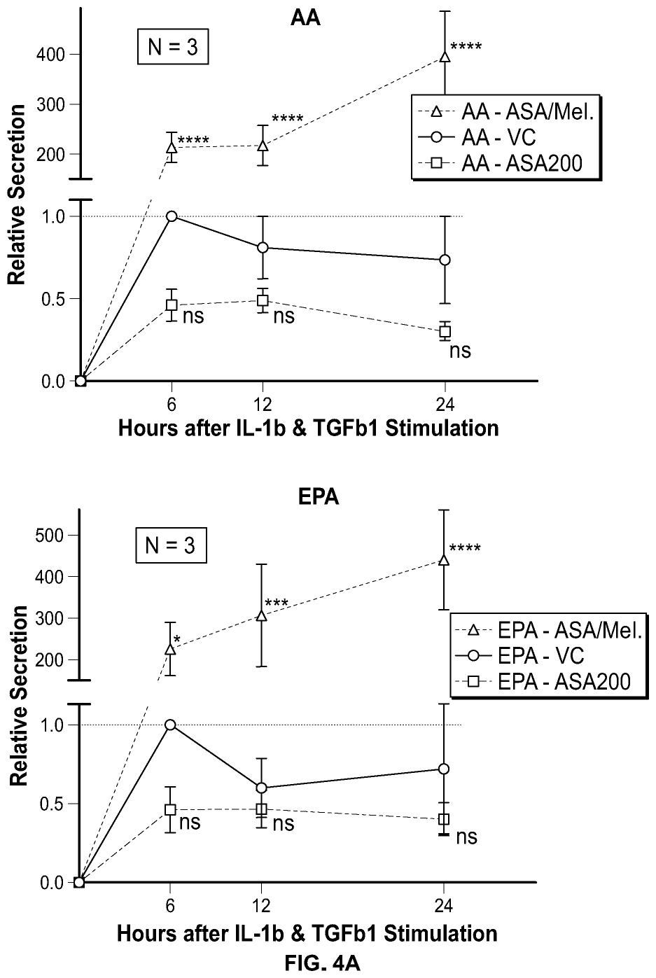

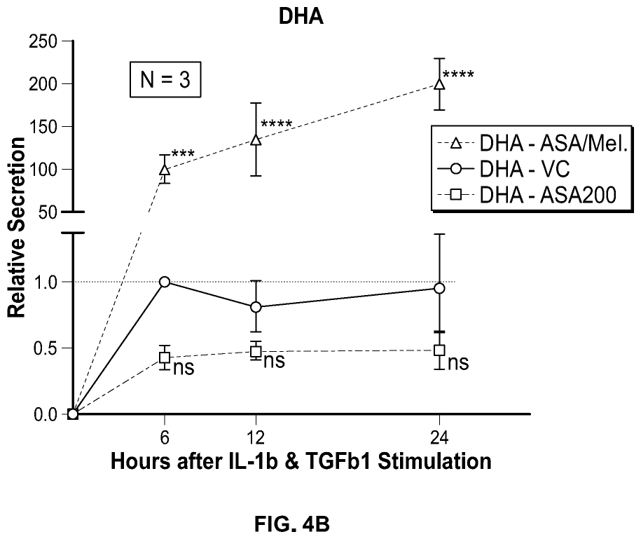

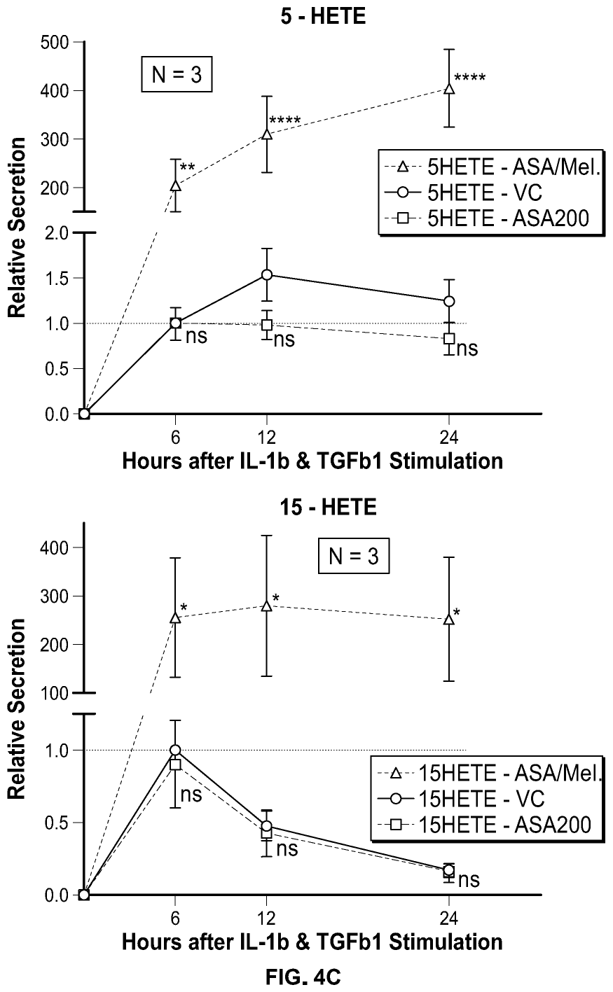

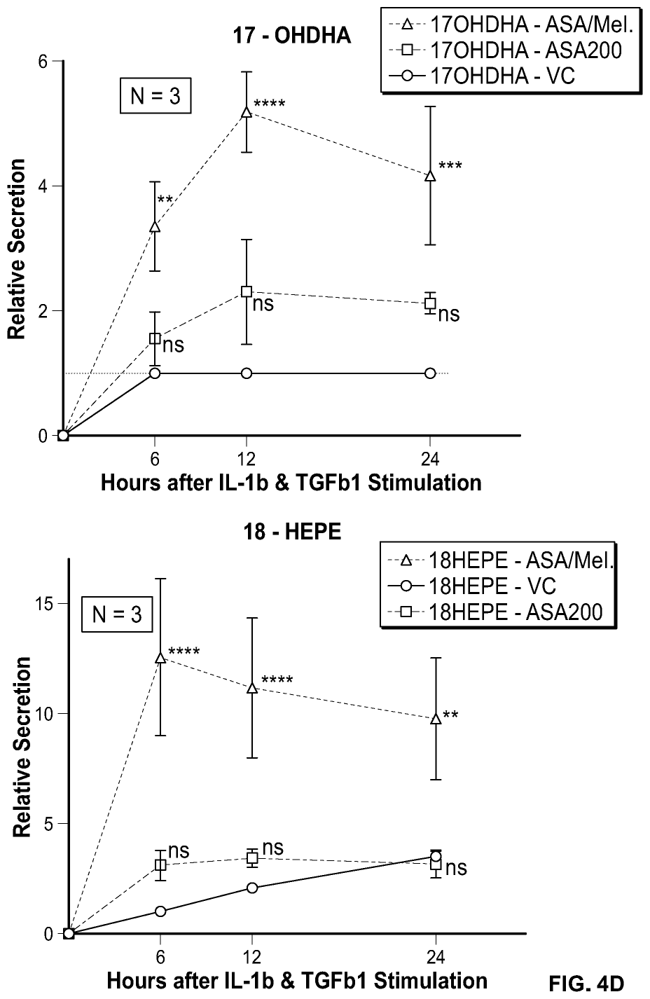

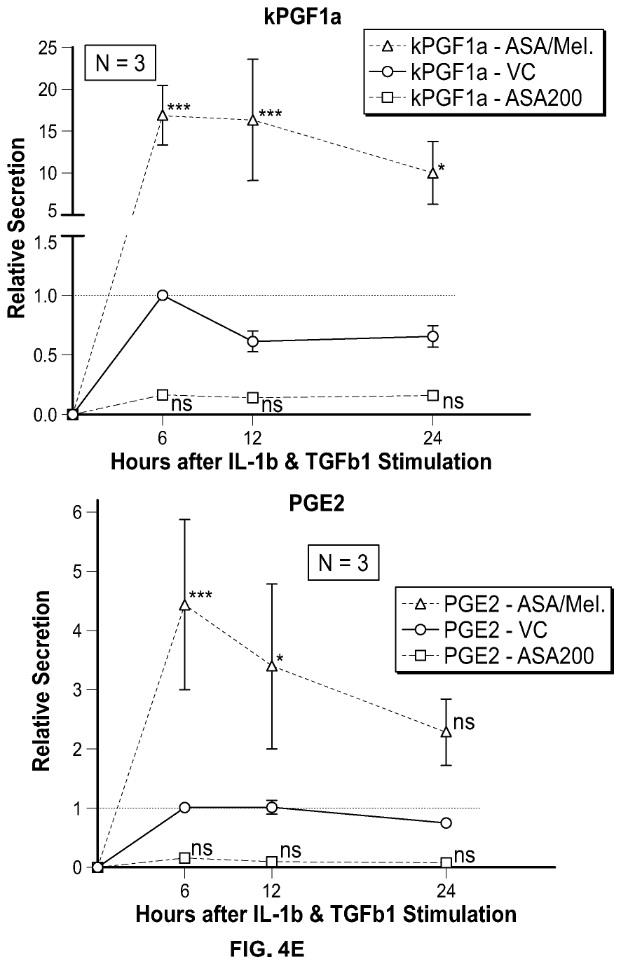

The relative secretion of polyunsaturated fatty acid (PUFA) precursors (AA, DHA and EPA), as well as relative secretion of acetylated-COX2 vs. COX2 products from HTCFs after an inflammatory stimulus and exposure to ASA combined with an extremely potent PLA2 agonist—melittin—was significantly greater than what was measured after exposure to either ASA alone or vehicle control (FIG. 4A-4E). When HTCFs were treated with ASA alone, there was no significant increase in relative secretion of precursor PUFAs (AA, DHA and EPA), however when ASA was applied in combination with melittin, there was a significant increase in their secretion—exemplifying melittin's ability to increase PUFA precursor availability for the downstream COX and LOX enzymes (FIGS. 4A and 4B). When HTCFs were treated with ASA alone, we observed no significant increase in the relative secretion of acetylated-COX2 products compared to the vehicle control, however there was a dramatically significant increase in secretion when ASA was applied in combination with melittin (FIGS. 4C and 4D)—exemplifying acetyl-COX2's ability to use the induced abundance of PUFA precursors to produce resolving mediators. ASA alone was able to decrease the secretion of COX2 products relative to vehicle control; however this effect was not statistically significant. ASA and melittin caused a significant increase in the secretion of COX2 products relative to the vehicle control (FIG. 4E), however this increase was an order of magnitude less than the increase in acetylated-COX2 products under the same culture conditions—exemplifying an overall shift in the net production of lipid mediators from pro-inflammatory COX2 products to the pro-resolving products of the LOX enzymes.

### Lipid Mediator Secretion Assay II

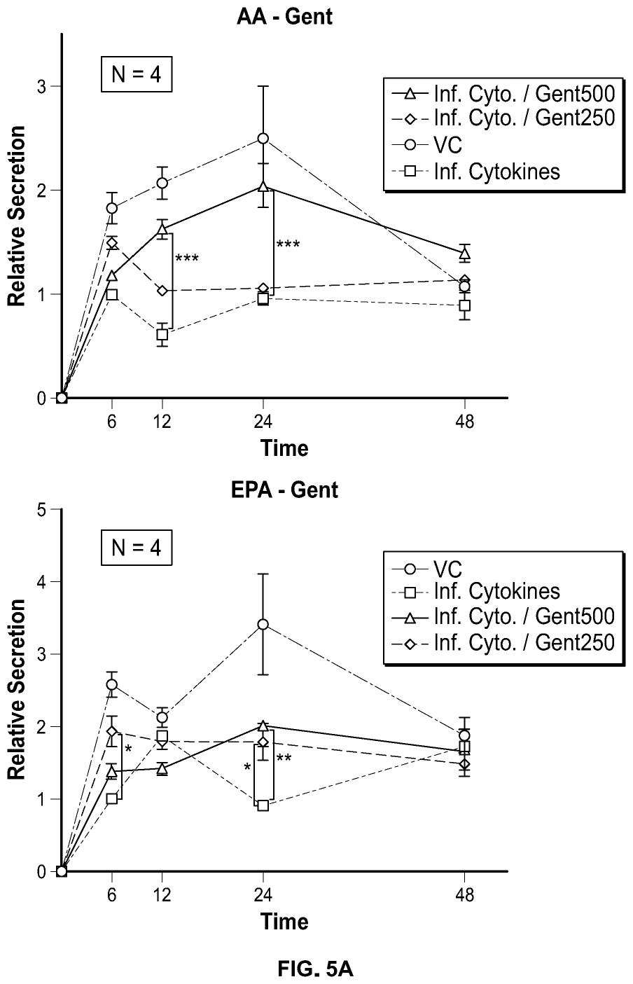

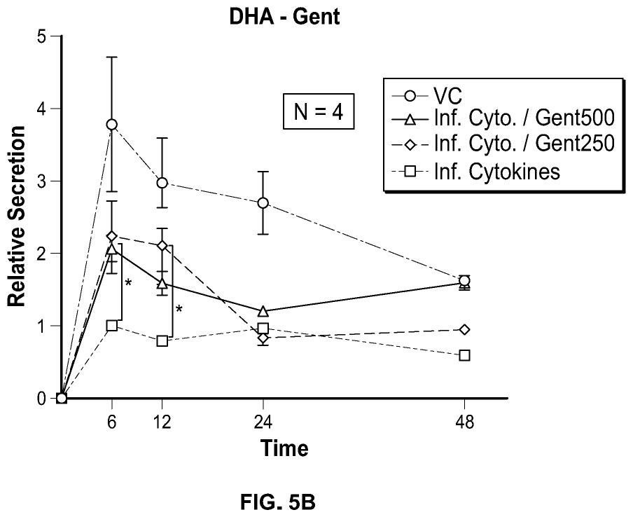

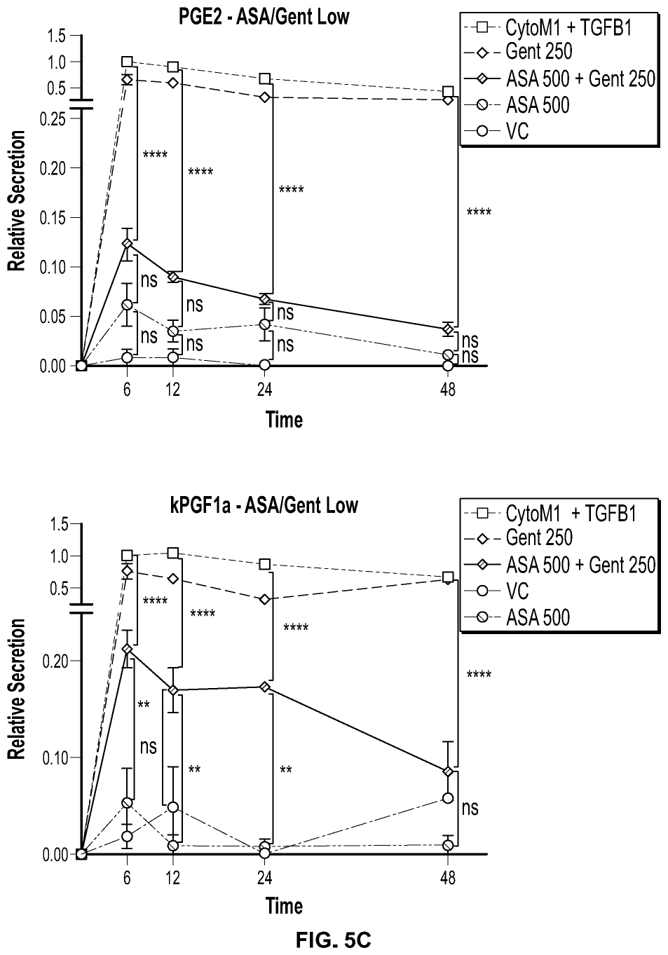

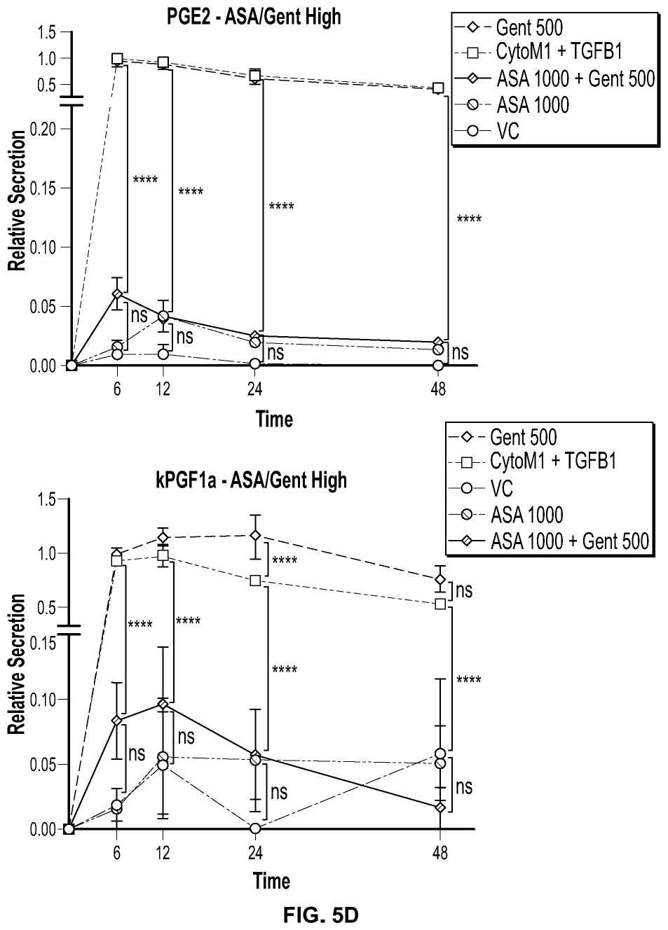

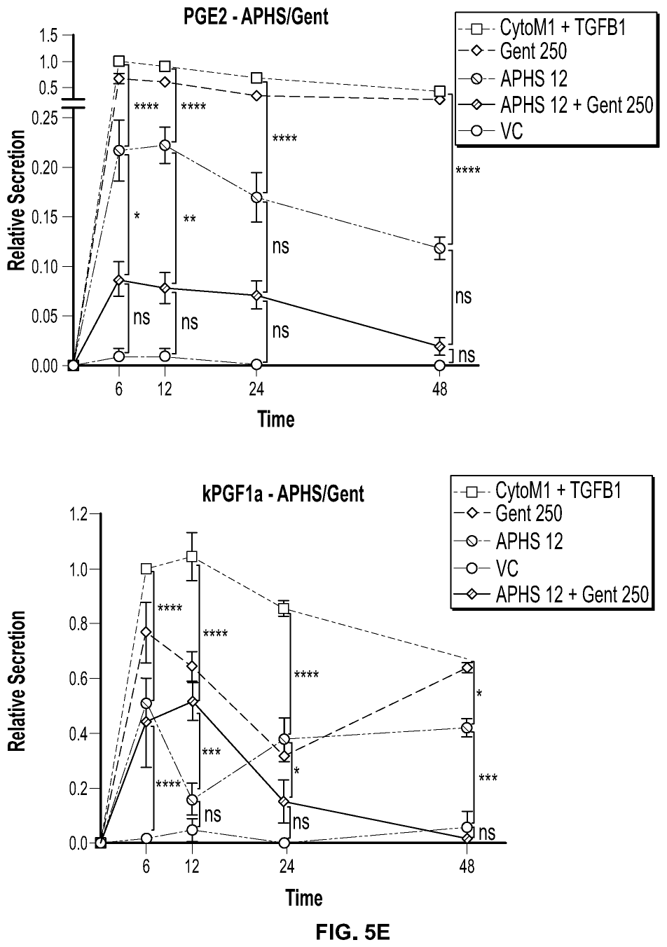

The relative secretion of PUFA precursors and COX2 products from HTCFs after an inflammatory stimulus and exposure to vehicle (negative control), CytoM1+TGFb1 (positive control), or CytoM1+TGFb1 with ASA, gentamicin, APHS, ASA/Gent or APHS/Gent was measured through LC-MS/MS analysis of cell culture supernatant at 6, 12, 24 and 48 hrs after experimental treatment. In inflammatory cytokine induced HTCFs, gentamicin was able to significantly increase the secretion of PUFA precursors: AA, EPA and DHA (FIGS. 5A and 5B). In a dose dependent manner, ASA was able to significantly inhibit CytoM1+TGFb1 induced COX2 activity, as measured through PGE2 and kPGF1a secretion (FIGS. 5C and 5D). Gentamicin had no relevant impact on this effect at any dose (FIGS. 5C and 5D). APHS was able to significantly inhibit COX2 activity, as measured through PGE2 and kPGF1a secretion (FIG. 5E). This exemplifies the greater specificity APHS has relative to ASA for acetylating Ser516 on COX2.

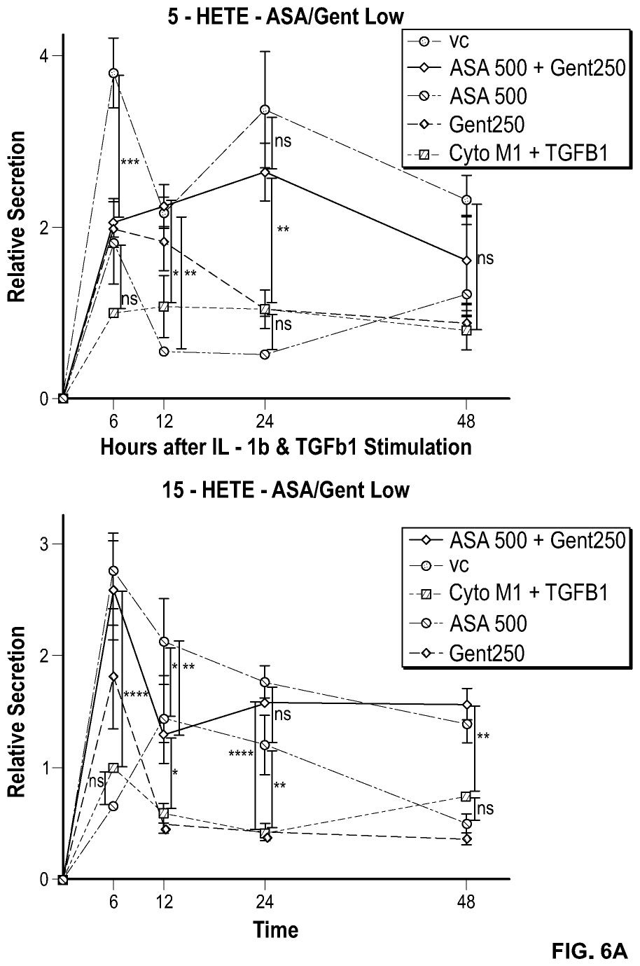

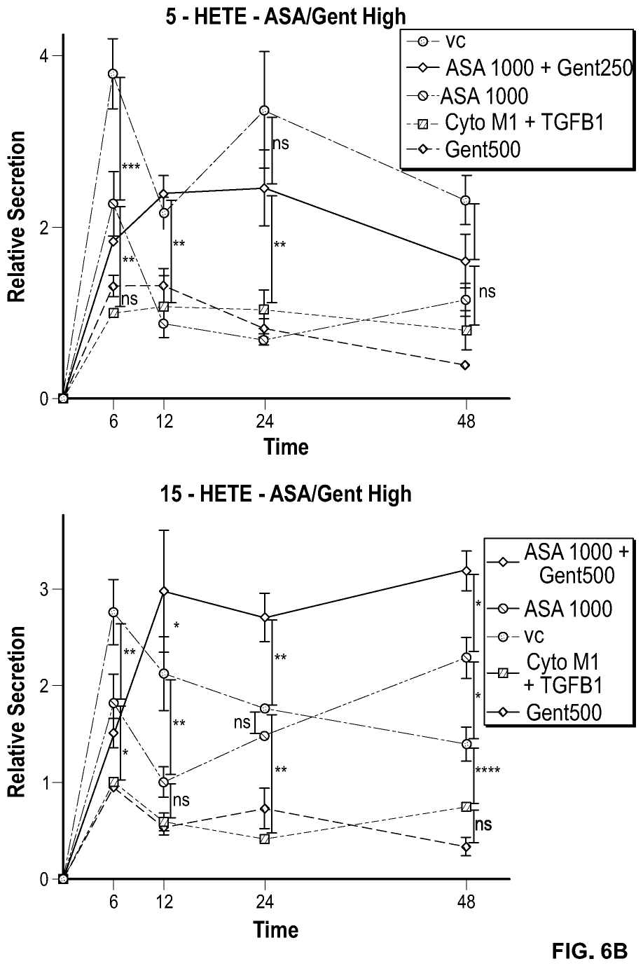

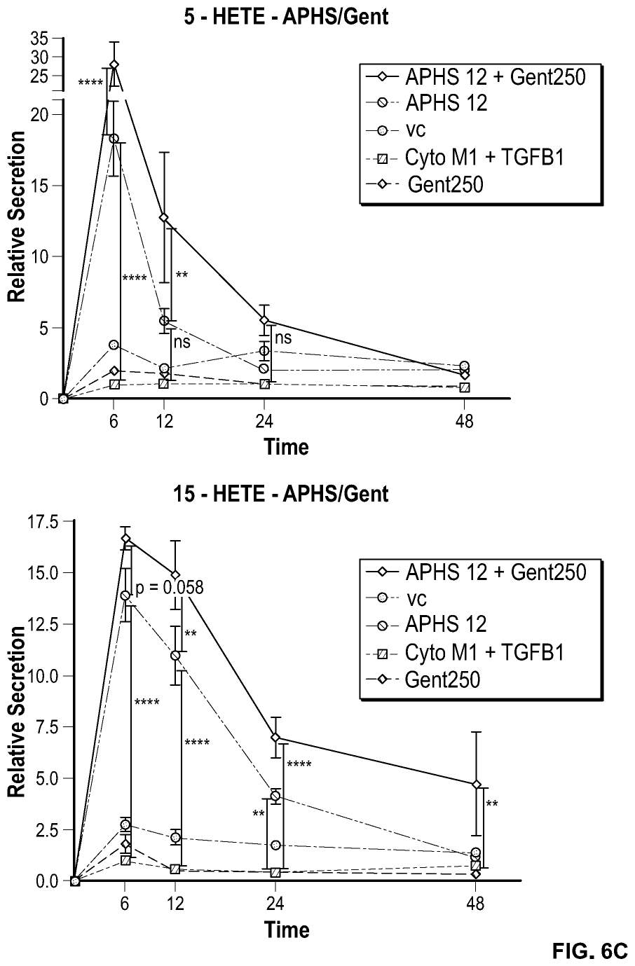

The relative secretion of acetylated-COX2 products from HTCFs after an inflammatory stimulus and exposure to vehicle (negative control), CytoM1+TGFb1 (positive control), or CytoM1+TGFb1 with ASA, gentamicin, APHS, ASA/Gent or APHS/Gent was measured through LC-MS/MS analysis of cell culture supernatant at 6, 12, 24 and 48 hrs after experimental treatment. In a dose dependent manner, ASA/Gent were able to significantly increase the secretion of acetylated-COX2 products relative HTCFs that had only been exposed to CytoM1+TGFb1, as measured through 5-HETE and 15-HETE secretion (FIGS. 6A and 6B). The secretion of acetyl-COX2 products by HTCFs exposed to ASA/Gent and CytoM1+TGFb1 either approached that, or exceeded that of the vehicle control group, indicating a mitigation of CytoM1+TGFb1's pro-inflammatory effects. ASA alone had either no effect or a significantly smaller effect on acetylated-COX2 products relative to the ASA/Gent experimental group. APHS profoundly and significantly increased the secretion of acetylated-COX2 products relative to all other treatment groups, this effect was significantly augmented by the addition of gentamicin (FIG. 6C). This exemplifies the greater specificity APHS has relative to ASA for acetylating Ser516 on COX2.

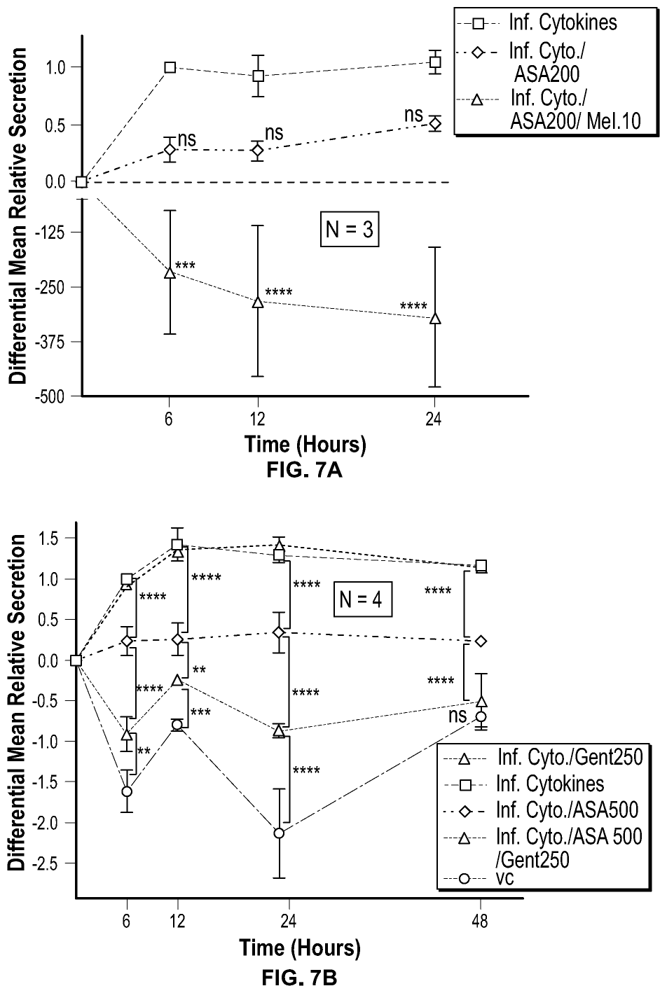

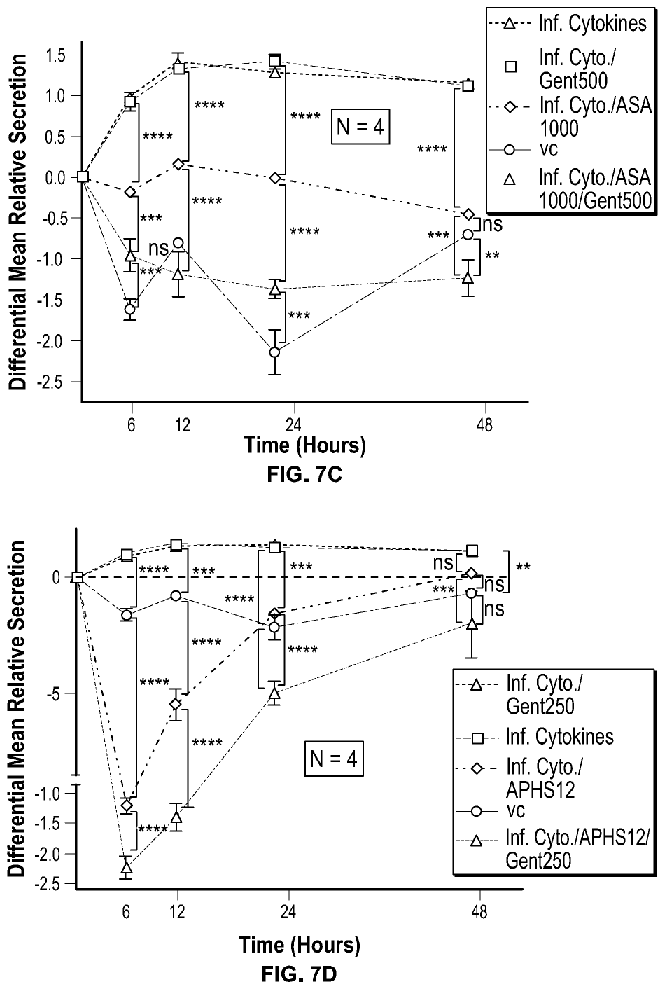

The relative differential enzymatic activity between acetylated-COX2 and COX2 within HTCFs after an inflammatory stimulus was calculated based on the mean relative secretion of PGE2/kPGF1a and 5-HETE/15-HETE under the indicated treatment conditions. ASA at 200 μg/ml was able to decrease the relative amount of COX2 products relative to inf. cytokines control, however with the addition of 10 μg/ml melittin, a significant shift to negative values for the COX2:acetyl-COX2 product ratio at all time points is observed (FIG. 7A).

In the second secretion assay, ASA at 500 μg/ml was able to significantly reduce the relative amount of COX2 products relative to the inf. cytokines control group, however the net production of lipid mediators still favored COX2, indicated by the positive values for the COX2:acetyl-COX2 ratio at all timepoints (FIG. 7B). The addition of 250 ul/ml of gentamicin significantly shifted the balance of enzymatic activity toward net acetyl-COX2 activity, as indicated by the predominantly negative values for the COX2:acetyl-COX2 ratio at all timepoints, almost mirroring the trend observed in the vehicle exposure group (FIG. 7B). Thus, gentamicin significantly augmented the activity of ASA.

ASA at 1000 μg/ml was able to significantly reduce the relative amount of COX2 products relative to the inf. cytokines control group, however it was unable to significantly shift the balance of enzymatic activity toward net actyl-COX2 production relative to COX2 production (FIG. 7C). The addition of 500 ul/ml of gentamicin significantly shifted the balance of enzymatic activity toward net acetyl-COX2 activity, as indicated by the predominantly negative values for the COX2:acetyl-COX2 ratio at all timepoints, mirroring the trend observed in the vehicle exposure group (FIG. 7C). Thus, gentamicin significantly augmented the activity of ASA.

APHS at 12 μg/ml was able to significantly shift the net enzymatic activity toward net acetylated-COX2 production of lipid mediators relative to the inf. cytokines control group. This effect was significantly augmented by the addition of 250 μg/ml gentamicin (FIG. 7D). This exemplifies the greater specificity APHS has relative to ASA for acetylating Ser516 on COX2.

### Collagen Contraction Assay II

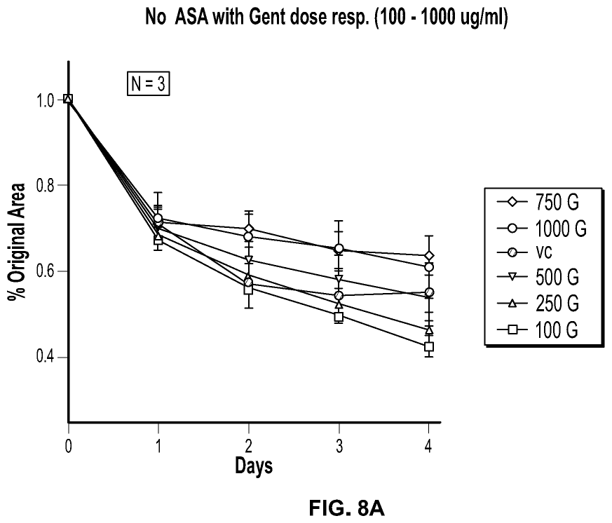

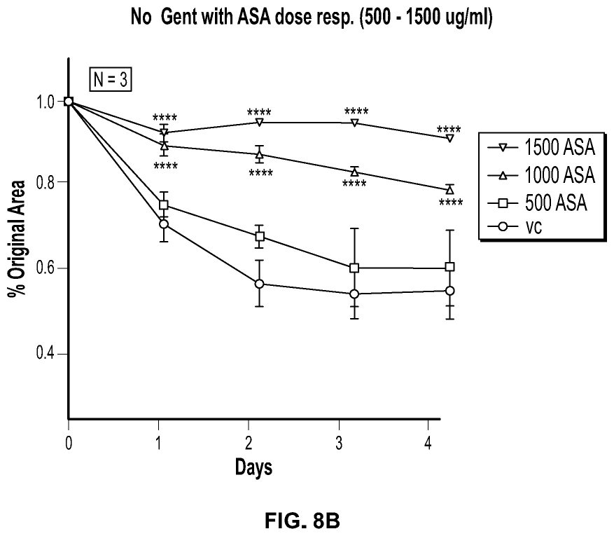

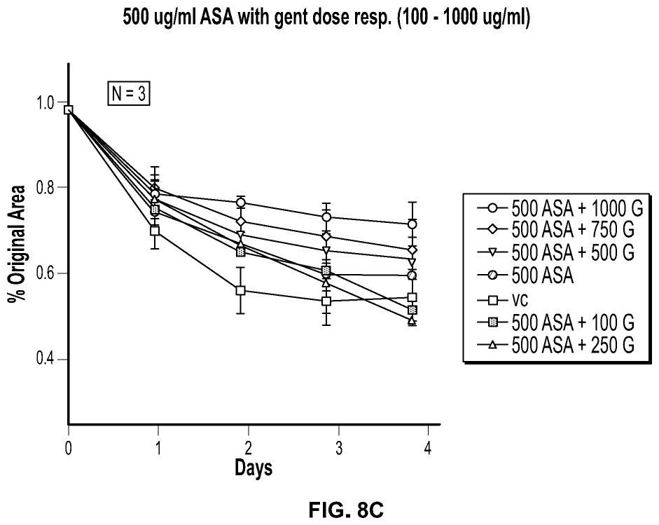

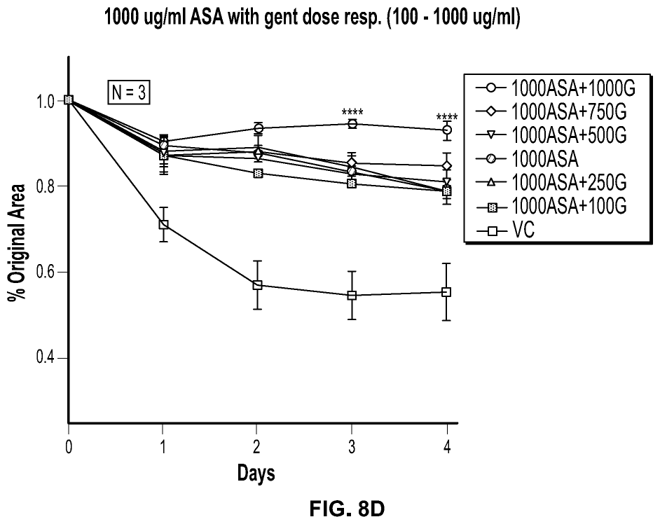

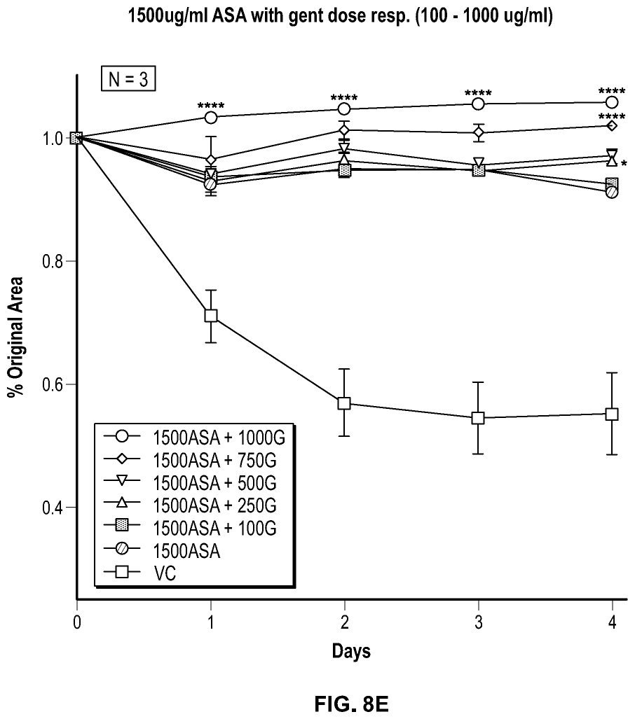

The delayed release fibroblast populated collagen lattice model was used to assess the dose dependent inhibition of HTCF-mediated collagen contraction by ASA and the augmentation of this effect in a dose dependent manner by gentamicin. Gentamicin alone had no significant effect on HTCF-mediated collagen contraction (FIG. 8A). ASA alone exhibited a dose dependent inhibition of HTCF-mediated collagen contraction (FIG. 8B). Gentamicin was unable to significantly increase the effect of ASA on HTCF-mediated collagen contraction at any of the tested concentrations (FIG. 8C). With higher concentrations of ASA (1000 and 1500 μg/ml), the augmenting effect of gentamicin becomes statistically apparent, in a dose dependent manner (FIGS. 8D and 8E).

### Collagen Contraction Assay III

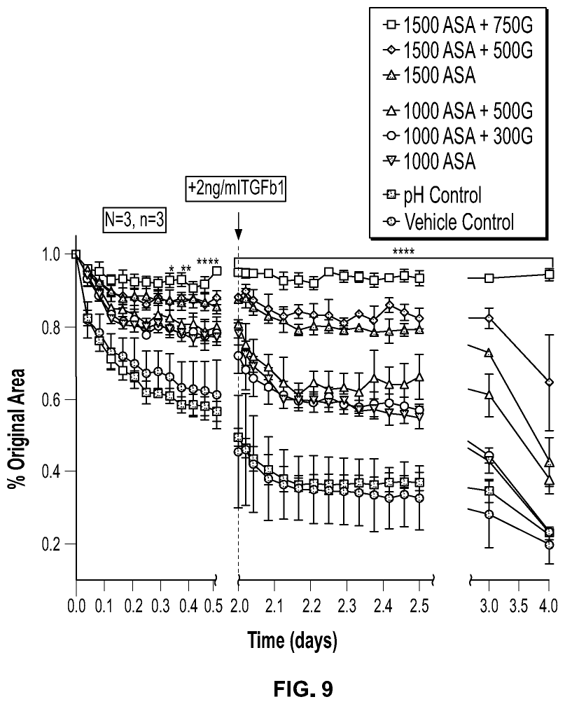

The delayed release fibroblast populated collagen lattice model was used to assess the dose dependent inhibition of HTCF-mediated collagen contraction by ASA and the augmentation of this effect in a dose dependent manner by gentamicin (FIG. 9). TGFb1 was added half way through the collagen contraction assay to assess the capacity of ASA/Gent to mitigate the TGFb1-induced, HTCF-mediated collagen contraction. This pro-scarring factor is known to be present in high amounts within the aqueous humor of glaucoma patients—a fluid that will be in contact with the surgical site for the remainder of the patient's life. We also included a pH control to assess the relative contribution of ASA's acidity to any observed effects—no significant effects due to pH were found. ASA was able to significantly impair HTCF-mediated contraction, at all concentrations assessed, both before and after stimulus with TGFb1. This effect was significantly augmented by the addition of 750 μg/ml of gentamicin. From this and the previous experiment, we can infer the most effective ratio of ASA to Gent to be at least 2:1.

To assess the relative cellular viability between different experimental conditions, an in situ fluorescence-based LIVE/DEAD assay was used (FIG. 10A-C). The relative proportion of living to total cells was unchanged relative to vehicle control by treatment with ASA, ASA/Gent, or by an acid equivalent to the concentration of ASA used (i.e., there were no significant differences between the treatment groups) (FIG. 10D). This suggests the effects seen in response to treatment are a modulation of cellular activity, rather than a cytotoxic effect that indirectly results in decreased HTCF activity overall.

### Collagen Contraction Assay IV

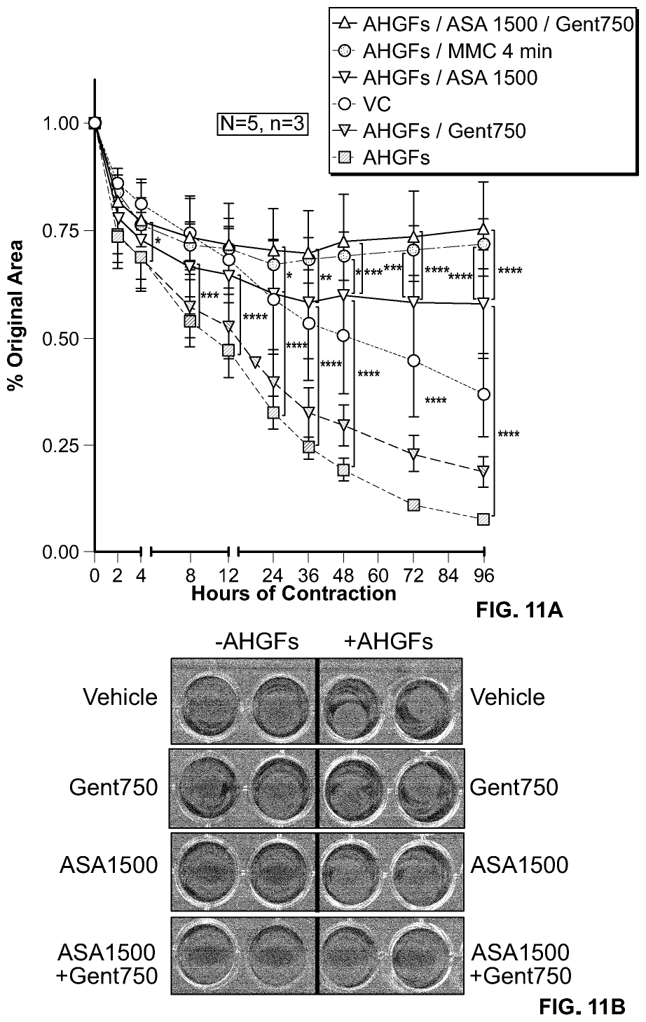

A modified delayed release fibroblast populated collagen lattice model was used to assess the effects of ASA and gentamicin on the inhibition of aqueous humor growth factor (AHGF) induced, HTCF-mediated collagen contraction—and compare the effect size to that of a current clinical gold standard, mitomycin C (FIG. 11). AHGFs significantly increase HTCF-mediated collagen contraction relative to vehicle control. ASA alone was able to significantly negate the effects of the AHGFs on HTCF-mediated collagen contraction, albeit significantly less than the current ophthalmic clinical gold standard for reducing inflammation-induced fibrosis—mitomycin C. The addition of gentamicin significantly augmented the effects of ASA, such that it mirrored the effect of mitomycin C. Gentamicin alone had no statistically significant effect on collagen contraction.

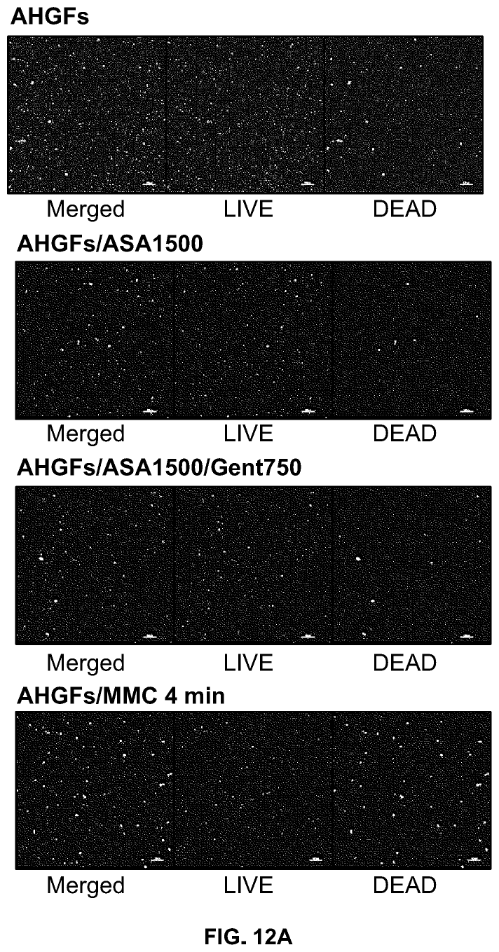

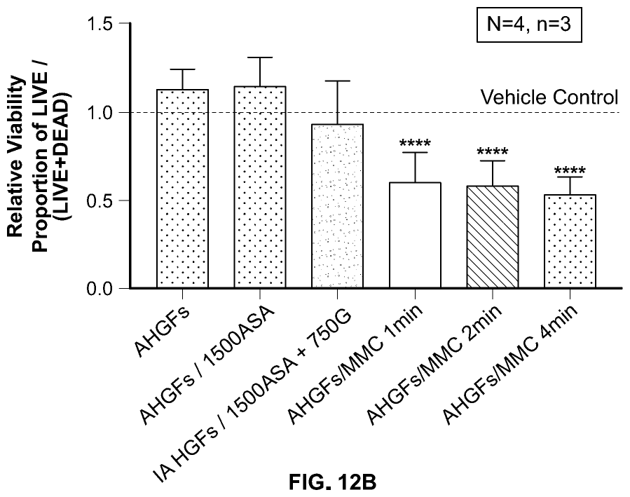

To assess the relative cellular viability between different experimental conditions, an in situ fluorescence-based LIVE/DEAD assay was used (FIG. 12A). The relative proportion of living to total cells was unchanged relative to vehicle control by treatment with ASA or ASA/Gent, however it was significantly reduced by all assessed exposures of mitomycin C (FIG. 12B). This indicates that the clinical gold standard, MMC, exerts anti-scarring/fibrotic effects through a cytotoxic mechanism, whereas ASA or ASA/Gent exert their effects through a non-cytotoxic mechanism.

### Collagen Contraction Assay V

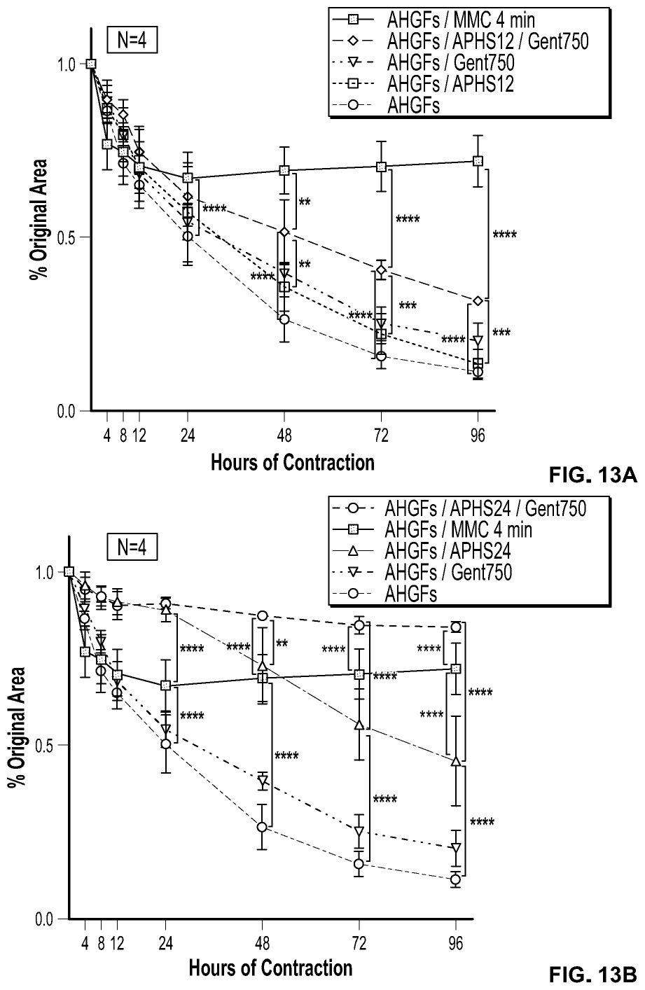

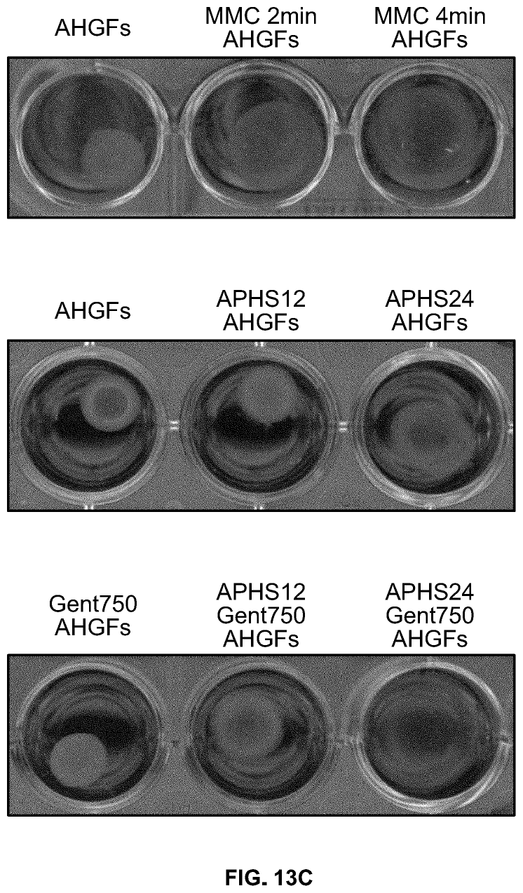

A modified delayed release fibroblast populated collagen lattice model was used to assess the effects of APHS and gentamicin on the inhibition of aqueous humor growth factor induced, HTCF-mediated collagen contraction—and compare the effect size to that of the current clinical gold standard, mitomycin C (FIG. 13A-C). In a dose dependent manner, APHS significantly inhibited AHGF-induced HTCF-mediated collagen contraction (FIGS. 13A and 13B). APHS 12 μg/ml with 750 μg/ml gentamicin inhibited AHGF-induced HTCF-mediated collagen contraction to a significantly greater degree than the clinical gold standard, mitomycin C.

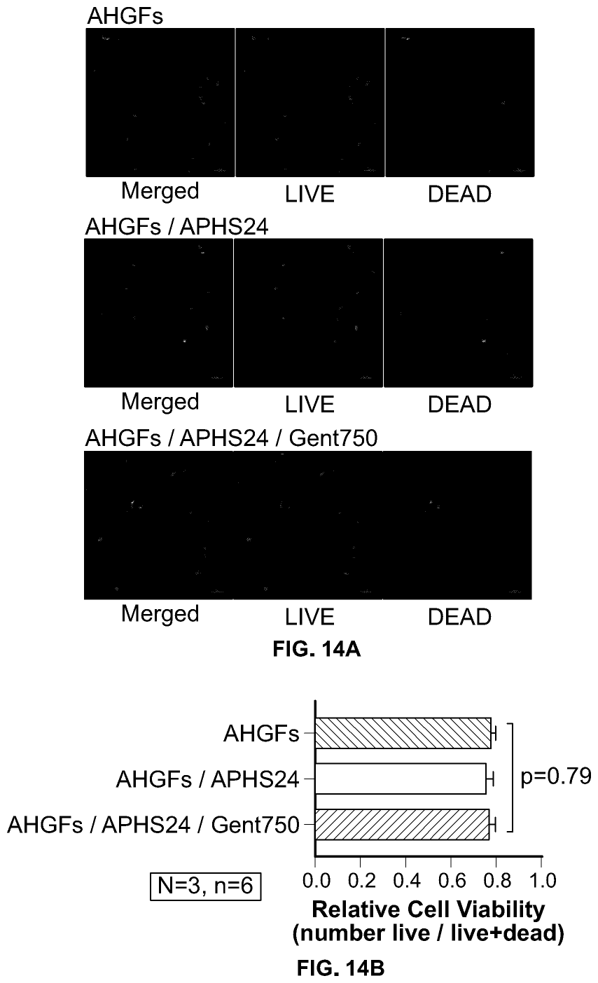

To assess the relative cellular viability between different experimental conditions, an in situ fluorescence-based LIVE/DEAD assay was used (FIG. 14A). The relative proportion of living to total cells was unchanged relative to the AHGFs control group by treatment with APHS or APHS/Gent (i.e., there were no significant differences between the treatment groups (FIG. 14B). This exemplifies APHS's non-cytotoxic mechanism of action.

Changes in Relative Metabolic Activity with Exposure to ASA or APHS

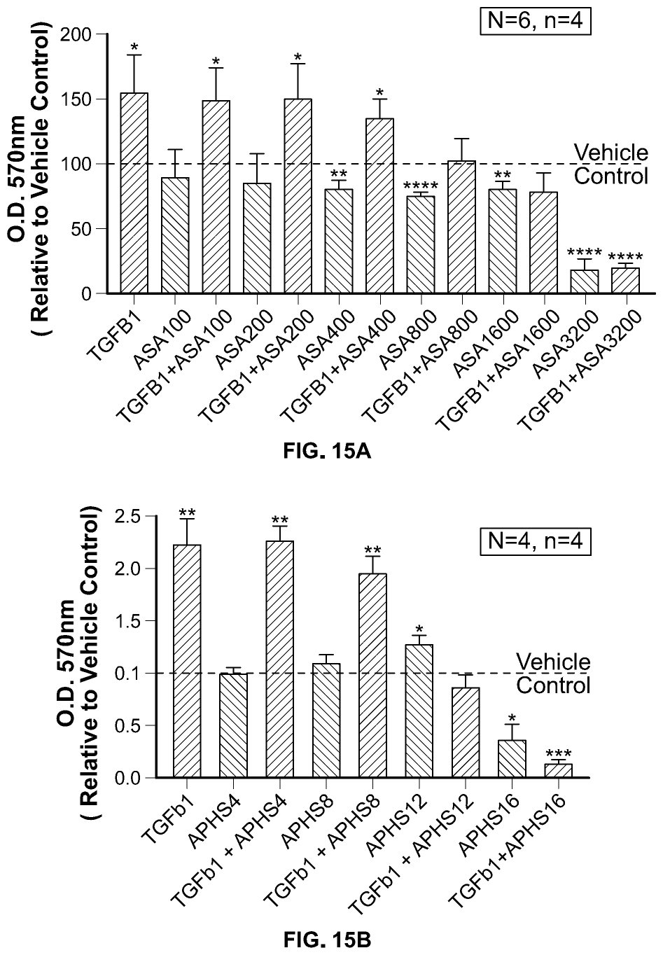

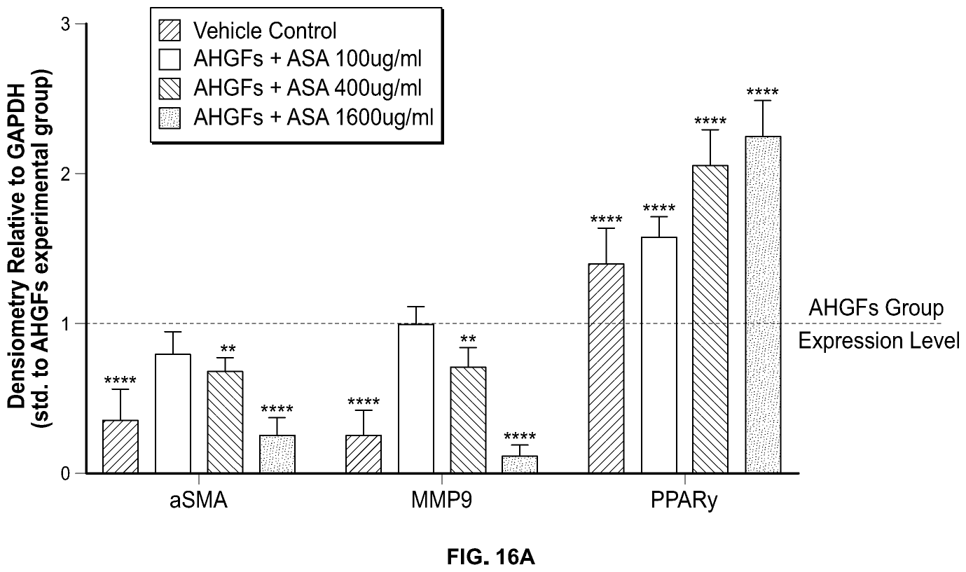

An MTT assay was used to assess the effects of COX2 acetylation on the relative metabolic activity of HTCFs and TGFb1-induced HTCFs (FIGS. 15A and 15B). Acetylation of COX2 in TGFb1-naïve HTCFs had relatively small effects on metabolic activity, until reaching the extreme of the dose range tested. Exposure to TGFb1 resulted in significantly heightened metabolic activity relative to vehicle control. In a dose dependent manner, both COX2 acetylators were able to mitigate the TGFb1-induced metabolic activity. This suggests that acetylation of COX2 can impair the increased metabolic activity of TGFb1-differentiated myofibroblasts.

Changes in Relative HTCF Protein Expression with Exposure to ASA

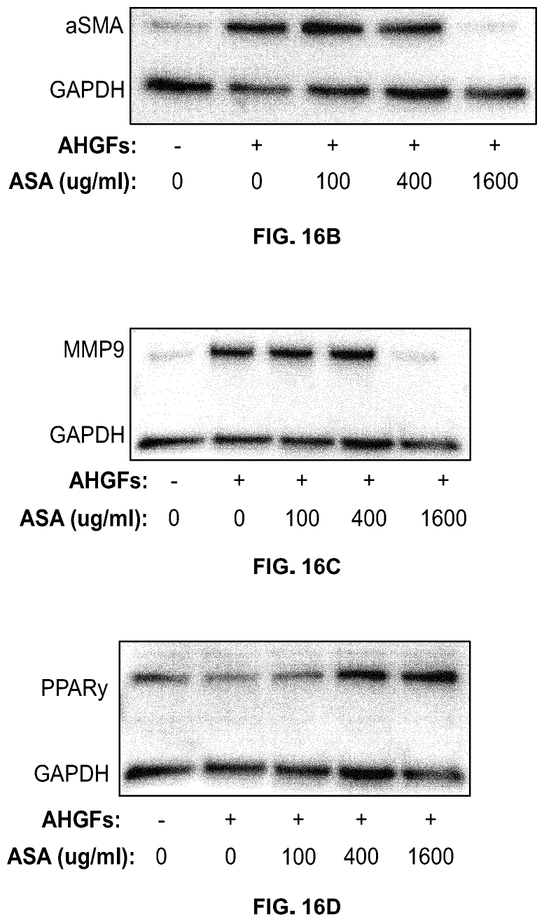

Western blot of total cellular protein lysate after experimental treatment was used to assess relative differences in AHGF-induced HTCF protein expression after exposure to various concentrations of ASA (FIG. 16A-D). In a dose dependent manner, ASA significantly impaired the AHGF-induced expression of αSMA (FIG. 16B) and MMP9 (FIG. 16C)—two proteins essential for fibrosis to occur. ASA was also able to significantly induce expression of PPARγ, which was significantly suppressed by AHGFs (FIG. 16D). PPARγ is a transcription factor with known anti-fibrotic actions [68, 69]. It exerts these effects partially through the suppression of the SMAD2/3-TGFb axis through upregulation of microRNA-145 and its subsequent knockdown of SMAD2/3 [70]. PPARγ is also a wide specificity receptor for several lipid mediators (e.g. 15-HETE) [71].

Changes in Relative HTCF Protein Expression with Exposure to ASA or APHS±Gent

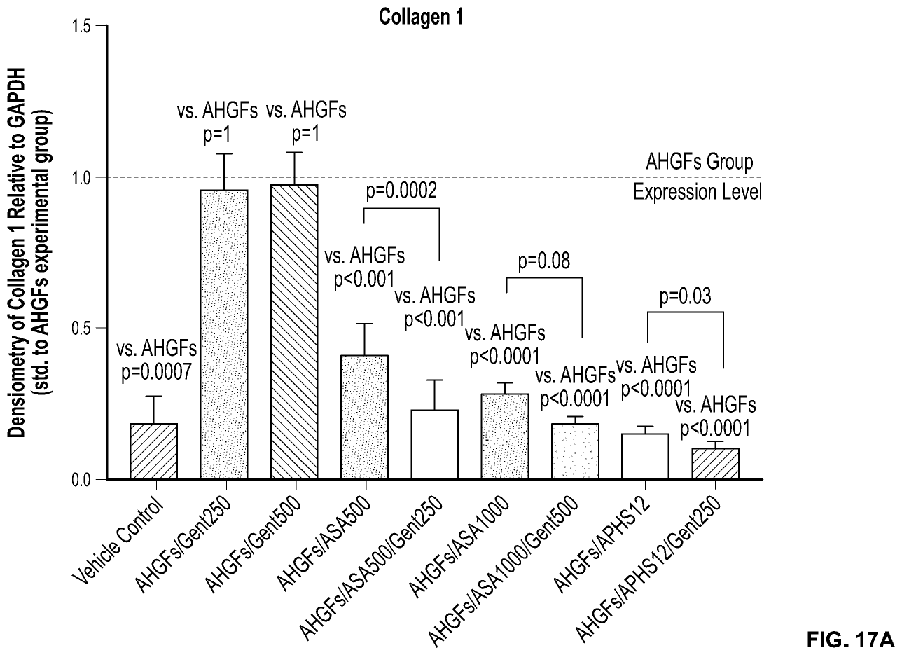

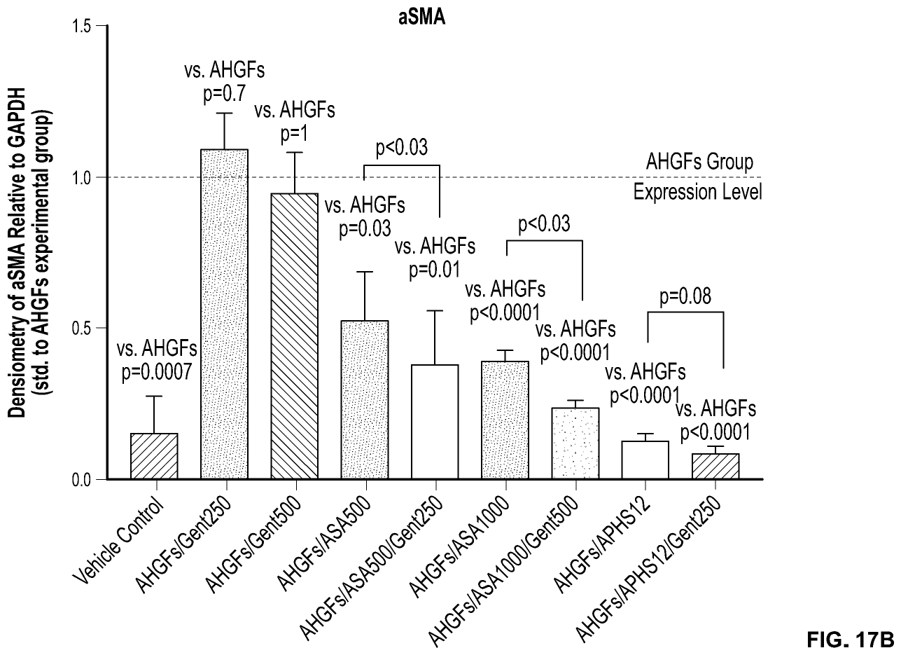

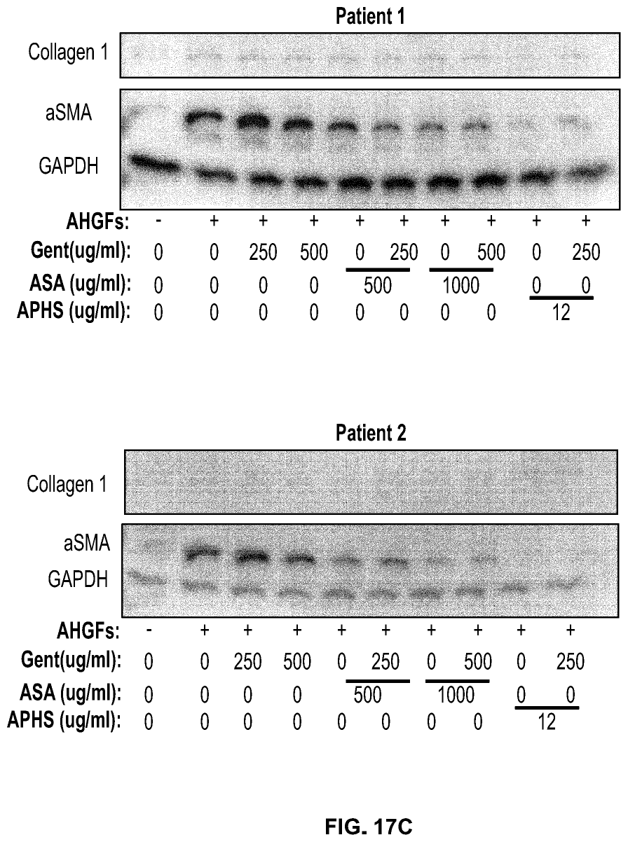

Western blot of total cellular protein lysate after experimental treatment was used to assess relative differences in AHGF-induced HTCF protein expression after exposure to various concentrations of ASA, APHS, ASA/Gent and APHS/Gent (FIG. 17A-C). The AHGF-induced expression of collagen 1 was significantly reduced by both ASA and APHS, with the addition of gentamicin significantly augmenting the observed effect (FIGS. 17A and 17C). The AHGF-induced expression of αSMA was significantly reduced by both ASA and APHS, with the addition of gentamicin significantly augmenting the observed effect (FIGS. 17B and 17C).

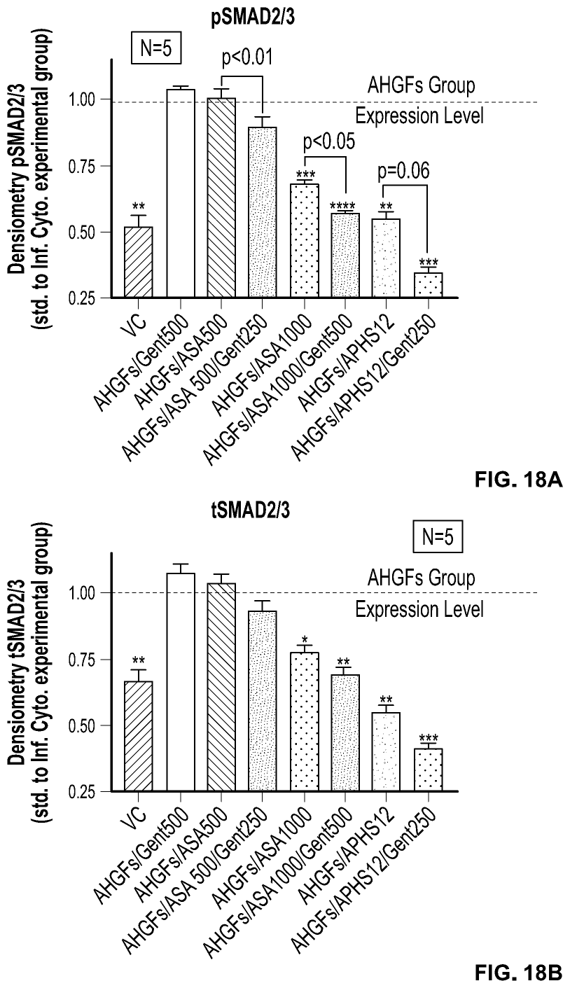

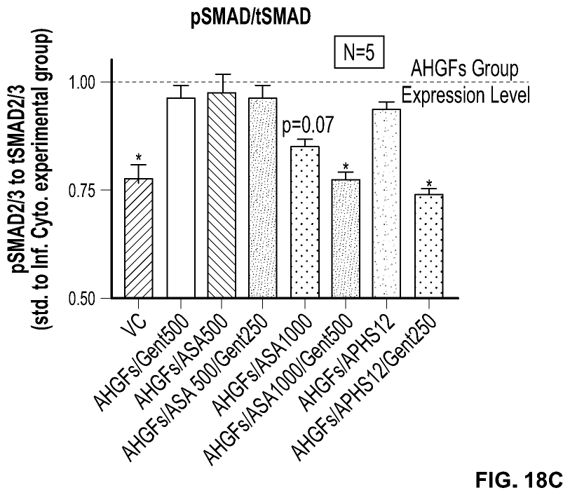

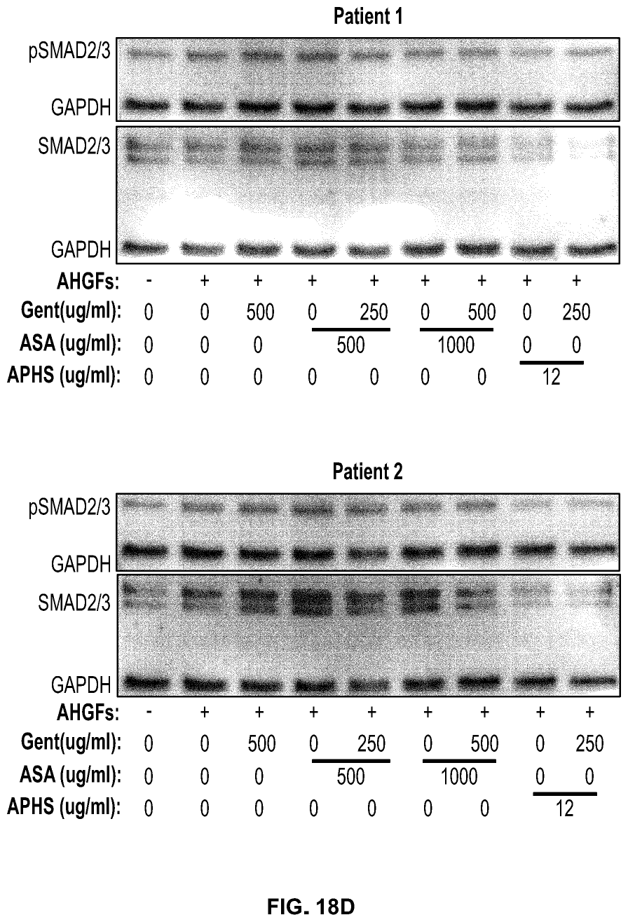

The relative expression of the transcription factor SMAD2/3 as well as its activated phosphorylated form (pSMAD2/3) were assessed through western blot (FIG. 18A-D). The AHGF-induced levels of pSMAD2/3 were significantly reduced by both ASA and APHS, with the addition of gentamicin significantly augmenting the observed effect (FIGS. 18A and 18D). The AHGF-induced expression of SMAD2/3 was significantly reduced by both ASA and APHS (FIGS. 18B and 18D). The ratio of pSMAD2/3 to tSMAD2/3 is an estimate of the proportion of total SMAD2/3 protein that is in its phosphorylated form. AHGFs significantly increased the proportion of pSMAD to tSMAD relative to vehicle control. Both ASA and APHS were able to significantly reduce the proportion of active to total SMAD2/3 from AHGF-induced levels (FIGS. 18C and 18D).

Changes in Relative HTCF Protein Expression with Application of Individual Acetyl-COX2 Products

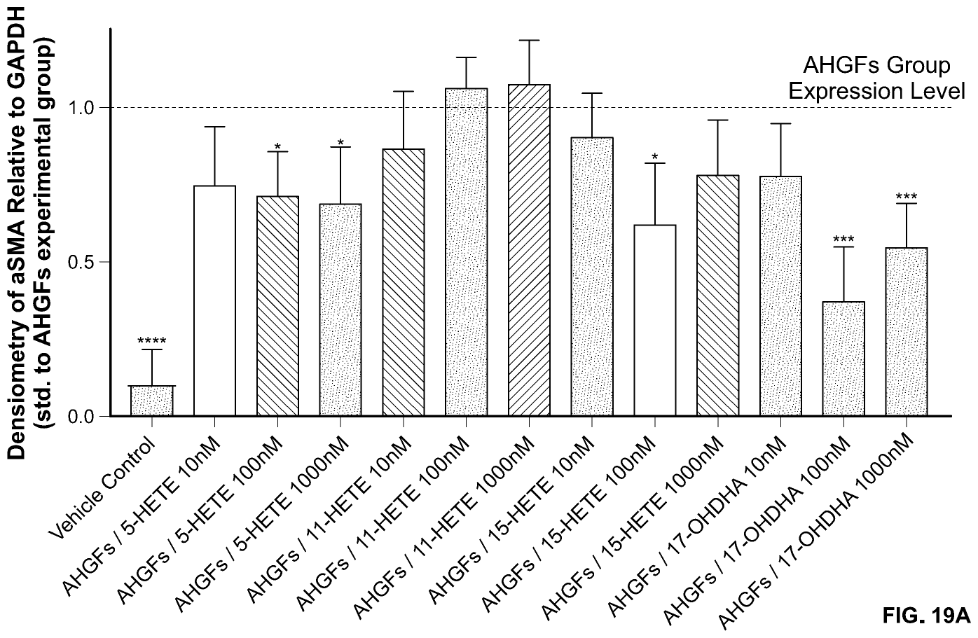

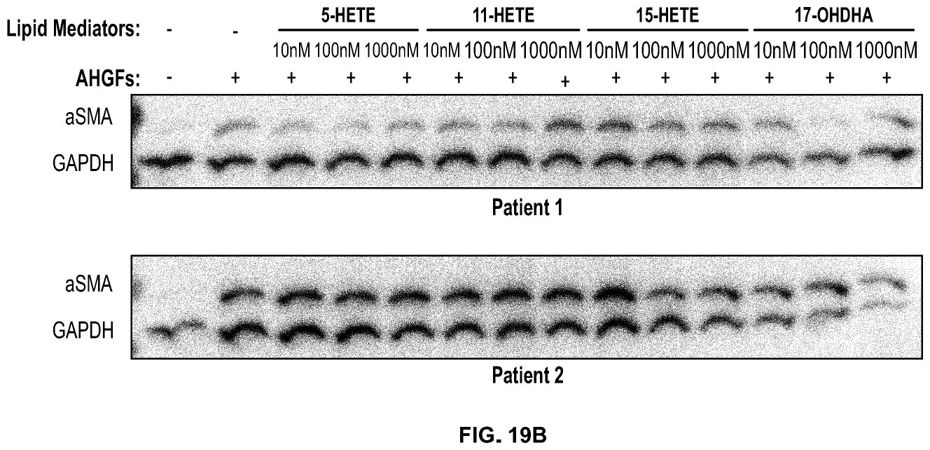

Western blot of total cellular protein lysate was used to assess relative differences in AHGF-induced HTCF protein expression after exposure to various concentrations of the lipid mediators that are produced in the presence of ASA/Gent or APHS/Gent (FIGS. 19A and 19B). The AHGF-induced expression of αSMA was significantly reduced by exposure to 5-HETE (100 and 1000 nM), 15-HETE (100 nM), as well as 17-OHDHA (100 and 1000 nM). Exposure to 11-HETE did not significantly affect AHGF-induced αSMA expression.

### EQUIVALENTS

Any examples provided herein are included solely for the purpose of illustrating the disclosure and are not intended to limit the disclosure in any way. Any drawings provided herein are solely for the purpose of illustrating various aspects of the disclosure and are not intended to be drawn to scale or to limit the disclosure in any way. Those skilled in the art will recognize, or be able to ascertain, using no more than routine experimentation, numerous equivalents to the specific embodiments described specifically herein. Such equivalents are intended to be encompassed in the scope of the claims appended hereto. The disclosures of all documents cited herein are incorporated herein by reference as if set forth in their entirety.

### CITED REFERENCES

[1] Hitchings R A, Grierson I. Clinico pathological correlation in eyes with failed fistulizing surgery. Trans Ophthalmol Soc U K 1983; 103:84.
[2] Mansouri K, Medeiros F A, Weinreb R N. Global rates of glaucoma surgery 2013. doi:10.1007/s00417-013-2464-7.
[3] Arora K S, Robin A L, Corcoran K J, Corcoran S L, Ramulu P Y. Use of Various Glaucoma Surgeries and Procedures in Medicare Bene fi ciaries from 1994 to 2012. Ophthalmology 2015; 122:1615-24. doi:10.1016/j.ophtha.2015.04.015.
[4] Mietz H, Raschka B, Krieglstein G K. Risk factors for failures of trabeculectomies performed without antimetabolites. Br J Ophthalmol 1999; 83:814-21. doi:10.1136/bjo.83.7.814.
[5] Fontana H, Nouri-Mahdavi K, Lumba J, Ralli M, Caprioli J. Trabeculectomy with Mitomycin C. Outcomes and Risk Factors for Failure in Phakic Open-Angle Glaucoma. Ophthalmology 2006; 113:930-6. doi:10.1016/j.ophtha.2006.01.062.
[6] Yamanaka O, Kitano-Izutani A, Tomoyose K, Reinach P S. Pathobiology of wound healing after glaucoma filtration surgery. BMC Ophthalmol 2015; 15 Suppl 1:157. doi:10.1186/s12886-015-0134-8.
[7] Zada M, Pattamatta U, White A. Modulation of Fibroblasts in Conjunctival Wound Healing. Ophthalmology 2017; 125:179-92. doi:10.1016/j.ophtha.2017.08.028.
[8] Shu D Y, Lovicu F J. Myofibroblast transdifferentiation: The dark force in ocular wound healing and fibrosis. Prog Retin Eye Res 2017. doi:10.1016/j.preteyeres.2017.08.001.
[9] Schein O D, Cassard S D, Tielsch J M, Gower E W. Cataract Surgery among Medicare Beneficiaries. Ophthalmic Epidemiol 2012; 19:257-64. doi:10.3109/09286586.2012.698692.
[10] Wormstone I M, Tamiya S, Anderson I, Duncan G. Transdifferentiation in the Human Lens Capsular Bag. Investig Ophthalmol Vis Sci 2002; 43.
[11] Symonds J G, Lovicu F J, Chamberlain C G. Differing effects of dexamethasone and diclofenac on posterior capsule opacification-like changes in a rat lens explant model. Exp Eye Res 2006; 83:771-82. doi:10.1016/j.exer.2006.03.017.
[12] Schaumberg D A, Dana M R, Christen W G, Glynn R J. A Systematic Overview of the Incidence of Posterior Capsule Opacification. Ophthalmology 1998; 105:1213-21.
[13] Ellwein L B, Friedlin I V, Mcbean I A M, Lee P P. Use of Eye Care Services among the 1991 Medicare Population. Ophthalmology 1991; 103:1732-43. doi:10.1016/S0161-6420(96)30433-8.
[14] Kato N, Shimmura S. Pterygium Epithelial-mesenchymal transition and the eye EMT markers in pterygium head epithelial cells Histopathology of eyebank corneas with pterygium. Inflamm Regen 2008; 23:434-9.
[15] Hwang J C. Regional Practice Patterns for Retinal Detachment Repair in the United States. Am J Ophthalmol 2012; 153:1125-8. doi:10.1016/j.ajo.2011.11.034.
[16] Shi H, Guo T, Liu P-C, Wang Q-Y, Du Y-R, Liu Q-Y, et al. Steroids as an adjunct for reducing the incidence of proliferative vitreoretinopathy after rhegmatogenous retinal detachment surgery: a systematic review and meta-analysis. Drug Des Devel Ther 2015; 9:1393-400.
[17] Leasher J L, Bourne R R A, Flaxman S R, Jonas J B, Keeffe J, Naidoo K, et al. Global Estimates on the Number of People Blind or Visually Impaired by Diabetic Retinopathy: A Meta-analysis From 1990 to 2010. Diabetes Care 2016; 39:1643-9. doi:10.2337/dc15-2171.
[18] Chiang P P, Lamoureux E L, Zheng Y, Tay W T, Mitchell P, Wang J J, et al. Frequency and risk factors of non-retinopathy ocular conditions in people with diabetes: the Singapore Malay Eye Study. Diabet Med 2012:32-40. doi:10.1111/dme.12053.
[19] Wick G, Grundtman C, Mayerl C, Wimpissinger T-F, Feichtinger J, Zelger B, et al. The Immunology of Fibrosis. vol. 31. 2013. doi:doi:10.1146/annurev-immunol-032712-095937.
[20] Tomasek J J, Gabbiani G, Hinz B, Chaponnier C, Brown R a. Myofibroblasts and mechano-regulation of connective tissue remodelling. Nat Rev Mol Cell Biol 2002; 3:349-63. doi:10.1038/nrm809.
[21] Darby I A, Zakuan N, Billet F, Desmouliere A. The myofibroblast, a key cell in normal and pathological tissue repair. Cell Mol Life Sci 2015:1-13. doi:10.1007/s00018-015-2110-0.
[22] Chang L, Crowston J G, Cordeiro M F, Akbar A N, Khaw P T. The role of the immune system in conjunctival wound healing after glaucoma surgery. Surv Ophthalmol 2000; 45:49-68. doi:10.1016/S0039-6257(00)00135-1.
[23] Asano S, Miyake K, Ota I, Sugita G, Kimura W, Sakka Y, et al. Reducing angiographic cystoid macular edema and blood-aqueous barrier disruption after small-incision phacoemulsification and foldable intraocular lens implantation. Multicenter prospective randomized comparison of topical diclofenac 0.1% and betamethason. J Cataract Refract Surg 2008; 34:57-63. doi:10.1016/j.jcrs.2007.08.030.
[24] Laurell C G, Zetterstrom C. Effects of dexamethasone, diclofenac, or placebo on the inflammatory response after cataract surgery. Br J Ophthalmol 2002; 86:1380-4. doi:10.1136/bjo.86.12.1380.
[25] Almeida D R P, Johnson D, Hollands H, Smallman D, Baxter S, Eng K T, et al. Effect of prophylactic nonsteroidal antiinflammatory drugs on cystoid macular edema assessed using optical coherence tomography quantification of total macular volume after cataract surgery. J Cataract Refract Surg 2008; 34:64-9. doi:10.1016/j.jcrs.2007.08.034.
[26] Zhao X, Xia S, Wang E, Chen Y. Comparison of the efficacy and patients' tolerability of Nepafenac and Ketorolac in the treatment of ocular inflammation following cataract surgery: A meta-analysis of randomized controlled trials. PLoS One 2017; 12:1-14. doi:10.1371/journal.pone.0173254.
[27] Sahu S, Ram J, Bansal R, Pandav S S, Gupta A. Effect of topical ketorolac 0.4%, nepafenac 0.1%, and bromfenac 0.09% on postoperative inflammation using laser flare photometry in patients having phacoemulsification. J Cataract Refract Surg 2015; 41:2043-8.
[28] Walters T, Endl M, Elmer T R, Levenson J, Majmudar P, Masket S. Sustained-release dexamethasone for the treatment of ocular inflammation and pain after cataract surgery. J Cataract Refract Surg 2015; 41:2049-59.
[29] Jones B M, Neville M W. Nepafenac: an ophthalmic nonsteroidal antiinflammatory drug for pain after cataract surgery. Ann Pharmacother 2013; 47:892-6.
[30] Duffin R M, Camras C B, Gardner S K, Pettit T H. Inhibitors of surgically induced miosis. Ophthalmology 1982; 89:966-79.
[31] Bali E, Feron E J, Peperkamp E, Veckeneer M, Mulder P G, Van Meurs J C. The effect of a preoperative subconjuntival injection of dexamethasone on blood-retinal barrier breakdown following scleral buckling retinal detachment surgery: A prospective randomized placebo-controlled double blind clinical trial. Graefe's Arch Clin Exp Ophthalmol 2010; 248:957-62. doi:10.1007/s00417-010-1319-8.
[32] Campochiaro P A. Pathogenic mechanisms in proliferative vitreoretinopathy. Arch Ophthalmol 1997; 115:237-41.
[33] Foster A, Resnikoff S. The impact of Vision 2020 on global blindness. Eye (Lond) 2005; 19:1133-5. doi:10.1038/sj.eye.6701973.
[34] Lee P, Wang C C, Adamis A P. Ocular Neovascularization: An Epidemiologic Review. Surv Ophthalmol 1998; 43.
[35] Chang J, Gabison E E, Kato T, Azar D T. Corneal neovascularization. Curr Opin Ophthalmol 2001; 12:242-9.
[36] Fini M E, Stramer B M. How the Cornea Heals Cornea-specific Repair Mechanisms Affecting Surgical Outcomes 2005; 33136:2-11.
[37] Chen W S, Cao Z, Leffler H, Nilsson U J, Panjwani N. Galectin-3 inhibition by a small-molecule inhibitor reduces both pathological corneal neovascularization and fibrosis. Investig Ophthalmol Vis Sci 2017; 58:9-20. doi:10.1167/iovs.16-20009.
[38] Perros P, Hegedus L, Bartalena L, Marcocci C, Kahaly G J, Baldeschi L, et al. Graves' orbitopathy as a rare disease in Europe: a European Group on Graves' Orbitopathy (EUGOGO) position statement. Orphanet J Rare Dis 2017; 12. doi:10.1186/s13023-017-0625-1.
[39] Dik W A, Virakul S, van Steensel L. Current perspectives on the role of orbital fibroblasts in the pathogenesis of Graves' ophthalmopathy. Exp Eye Res 2016; 142:83-91. doi:10.1016/j.exer.2015.02.007.
[40] Tham Y C, Li X, Wong T Y, Quigley H A, Aung T, Cheng C Y. Global prevalence of glaucoma and projections of glaucoma burden through 2040: A systematic review and meta-analysis. Ophthalmology 2014; 121:2081-90. doi:10.1016/j.ophtha.2014.05.013.
[41] Klein R, Klein B E K, Knudtson M D, Meuer S M, Swift M, Gangnon R E. Fifteen-Year Cumulative Incidence of Age-Related Macular Degeneration The Beaver Dam Eye Study. Ophthalmology 2007:253-62. doi:10.1016/j.ophtha.2006.10.040.
[42] Daniel E, Toth C A, Grunwald J E, Jaffe G J, Martin D F, Fine S L, et al. Risk of Scar in the Comparison of Age-related Macular Degeneration Treatments Trials. Ophthalmology 2014; 121:656-66. doi:10.1016/j.ophtha.2013.10.019.
[43] Daniel E, Pan W, Ying G shuang, Kim B J, Grunwald J E, Ferris F L, et al. Development and Course of Scars in the Comparison of Age-Related Macular Degeneration Treatments Trials. Ophthalmology 2018; 125:1037-46. doi:10.1016/j.ophtha.2018.01.004.
[44] Stein J, Newman-Casey P, Mrinalini T, Lee P, Hutton D. Cost-Effectiveness of Bevacizumab and Ranibizumab for Newly Diagnosed Neovascular Macular Degeneration. Ophthalmology 2015; 85:1-27. doi:10.1016/j.neuroimage.2013.08.045. The.
[45] Schmier J K, Covert D W, Lau E C. Patterns and costs associated with progression of age-related macular degeneration. Am J Ophthalmol 2012; 154:675-81. doi:10.1016/j.ajo.2012.04.017.
[46] Kalgutkar A S, Crews B C, Rowlinson S W, Garner C, Cyclooxygenase-I, Kalgutkar A S, et al. Aspirin-like Molecules that Covalently Inactivate Cyclooxygenase-2. Science (80-) 1998; 280:1268-70.
[47] Kalgutkar A S, Kozak K R, Crews B C, Hochgesang G P, Marnett L J. Covalent modification of cyclooxygenase-2 (COX-2) by 2-acetoxyphenyl alkyl sulfides, a new class of selective COX-2 inactivators. J Med Chem 1998; 41:4800-18. doi:10.1021/jm980303s.
[48] Hochgesang G P, Nemeth-Cawley J F, Rowlinson S W, Caprioli R M, Marnett L J. Functional analysis of the molecular determinants of cyclooxygenase-2 acetylation by 2-acetoxyphenylhept-2-ynyl sulfide. Arch Biochem Biophys 2003; 409:127-33. doi:10.1016/S0003-9861(02)00549-0.
[49] Chiang N, Bermudez E A, Ridker P M, Hurwitz S, Serhan C N. Aspirin triggers antiinflammatory 15-epi-ipoxin A4 and inhibits thromboxane in a randomized human trial. Proc Natl Acad Sci 2004; 101:15178-83. doi:10.1073/pnas.0405445101.
[50] Rowlinson S W, Crews B C, Goodwin D C, Schneider C, Gierse J K, Marnett L J. Spatial Requirements for 15-(R)-Hydroxy-5 Z, 8 Z, 11 Z, 13 E—eicosatetraenoic Acid Synthesis within the Cyclooxygenase Active Site of Murine COX-2. J Biol Chem 2000; 275:6586-91.
[51] Martinez-salgado C, Rodriguez-barbero A, Lema P D E, Lopez-novoal J M, Reina V, Investigation S De, et al. Involvement of phospholipase A2 in gentamicin-induced rat mesangial cell activation. Am J Physiol Physiol 1997; 273:F60-6.
[52] Argiolas A, Pisano J J. Facilitation of phospholipase A2 activity by mastoparans, a new class of mast cell degranulating peptides from wasp venom. J Biol Chem 1983; 258:13697-702.
[53] Steiner M R, Bomalaski J S, Clark M A. Responses of purified phospholipases A, to phospholipase A2 activating protein (PLAP) and melittin. Biochem Biophys Acta 1993; 1166:124-30.
[54] Markaverich B M, Crowley J, Rodriquez M, Shoulars K, Thompson T. Tetrahydrofurandiol stimulation of phospholipase A2, lipoxygenase, and cyclooxygenase gene expression and MCF-7 human breast cancer cell proliferation. Environ Health Perspect 2007; 115:1727-31. doi:10.1289/ehp.10659.
[55] Cajal Y, Jain M K. Synergism between mellitin and phospholipase A2 from bee venom: Apparent activation by intervesicle exchange of phospholipids. Biochemistry 1997; 36:3882-93. doi:10.1021/bi962788×.
[56] Serhan C N, Savill J. Resolution of inflammation: the beginning programs the end. Nat Immunol 2005; 6:1191-7. doi:10.1038/ni1276.
[57] Serhan C N, Chiang N, Dalli J. The resolution code of acute inflammation: Novel pro-resolving lipid mediators in resolution. Semin Immunol 2015; 27:200-15. doi:10.1016/j.smim.2015.03.004.
[58] Dallon J C, Ph D, Ehrlich H P. A Review of Fibroblast Populated Collagen Lattices 2008:1-20.
[59] Schmitz N, Laverty S, Kraus V B, Aigner T. Basic methods in histopathology of joint tissues. Osteoarthr Cartil 2010; 18:S113-6. doi:10.1016/j.joca.2010.05.026.
[60] Whittaker P, Kloner R A, Boughner D R, Pickering J G. Quantitative assessment of myocardial collagen with picrosirius red staining and circularly polarized light. Basic Res Cardiol 1994; 89:397-410. doi:10.1007/BF00788278.
[61] Eberth J F, Gresham V C, Reddy A K, Popovic N, Wilson E, Humphrey J D. Importance of pulsatility in hypertensive carotid artery growth and remodeling. J Hypertens 2009; 27:2010-21. doi:10.1097/HJH.0b013e32832e8dc8.
[62] How A, Chua J L L, Charlton A, Su R, Lim M, Kumar R S, et al. Combined treatment with bevacizumab and 5-Fluorouracil attenuates the postoperative scarring response after experimental glaucoma filtration surgery. Investig Ophthalmol Vis Sci 2010; 51:928-32. doi:10.1167/iovs.09-3949.
[63] Rich L, Whittaker P. Collagen and Picrosirius Red Staining: a Polarized Light Assessment of Fibrillar Hue and Spatial Distribution. Braz J Morphol Sci 2005; 22:97-104.
[64] Sun X, Kaufman P D. Ki-67: more than a proliferation marker. Chromosoma 2018:1-12.
[65] Hinz B. Formation and function of the myofibroblast during tissue repair. J Invest Dermatol 2007; 127:526-37. doi:10.1038/sj.jid.5700613.
[66] Le Faouder P, Baillif V, Spreadbury I, Motta J P, Rousset P, Chene G, et al. LC-MS/MS method for rapid and concomitant quantification of pro-inflammatory and pro-resolving polyunsaturated fatty acid metabolites. J Chromatogr B Anal Technol Biomed Life Sci 2013; 932:123-33. doi:10.1016/j.jchromb.2013.06.014.
[67] Martin-venegas R, Olga J, Jose J. Liquid chromatography-tandem mass spectrometry analysis of eicosanoids and related compounds in cell models 2014; 964:41-9. doi:10.1016/j.jchromb.2014.05.024.
[68] Luo Y H, Ouyang P B, Tian J, Guo X J, Duan X C. Rosiglitazone inhibits TGF-β 1 induced activation of human tenon fibroblasts via p38 signal pathway. PLoS One 2014. doi:10.1371/journal.pone.0105796.
[69] Kuriyan A E, Lehmann G M, Kulkarni A A, Woeller C F, Feldon S E, Hindman H B, et al. Electrophilic PPARγ ligands inhibit corneal fibroblast to myofibroblast differentiation invitro: A potentially novel therapy for corneal scarring. Exp Eye Res 2012. doi:10.1016/j.exer.2011.11.018.
[70] Zhu H Y, Li C, Zheng Z, Zhou Q, Guan H, Su L L, et al. Peroxisome proliferator-activated receptor-γ (PPAR-γ) agonist inhibits collagen synthesis in human hypertrophic scar fibroblasts by targeting Smad3 via miR-145. Biochem Biophys Res Commun 2015. doi:10.1016/j.bbrc.2015.02.061.
[71] Croasdell A, Duffney P F, Kim N, Lacy S H, Sime P J, Phipps R P. PPAR γ and the Innate Immune System Mediate the Resolution of Inflammation. PPAR Res 2015. doi:10.1155/2015/549691.

We claim:

1. A pharmaceutical composition comprising a cyclooxygenase 2 (COX2) Ser516 acetylating agent and a cytosolic phospholipase A2 (cPLA2) agonist.

2. The pharmaceutical composition of claim 1, wherein the COX2 Ser516 acetylating agent is acetylsalicylic acid (ASA) or a 2-acetoxyphenyl alkyl sulfide.

3. The pharmaceutical composition of claim 2, wherein the 2-acetoxyphenyl alkyl sulfide is o-(acetoxyphenyl)hept-2-ynyl sulfide (APHS).

4. The pharmaceutical composition of any one of claims 1-3, wherein the cPLA2 agonist is gentamicin, tobramycin, mastoparan, phospholipase A2 activating protein (PLAP), tetrahydrofurandiol or melittin.

5. The pharmaceutical composition of any one of claims 1-4, further comprising a pharmaceutically acceptable carrier, diluent or excipient.

6. The pharmaceutical composition of any one of claims 1-5, wherein the composition is formulated for ocular administration.

7. A method of decreasing ocular inflammation in a subject, comprising administering to the subject a COX2 Ser516 acetylating agent and a cPLA2 agonist.

8. A method of decreasing ocular fibrosis and/or scarring in a subject, comprising administering to the subject a COX2 Ser516 acetylating agent and a cPLA2 agonist.

9. A method of decreasing ocular collagen contraction and/or remodelling in a subject, comprising administering to the subject a COX2 Ser516 acetylating agent and a cPLA2 agonist.

10. A method of decreasing ocular fibroblast cellular proliferation in a subject, comprising administering to the subject a COX2 Ser516 acetylating agent and a cPLA2 agonist.

11. The method of claim 10, wherein the fibroblast is a myofibroblast.

12. The method of claim 10 or 11, wherein the fibroblast is a human Tenon's capsule fibroblast (HTCF).

13. The method of any one of claims 7-12, wherein the ocular inflammation is inflammation of the conjunctiva.

14. The method of claim 13, wherein the inflammation of the conjunctiva is caused by conjunctivitis.

15. The method of claim 14, wherein the conjunctivitis is allergic conjunctivitis.

16. The method of any one of claim 7-12, wherein the COX2 Ser516 acetylating agent and the cPLA2 agonist are administered before, during and/or after ocular surgery.

17. The method of claim 16, wherein the ocular surgery comprises one or more of:
a. manipulation of the conjunctiva and/or Tenons;
b. a conjunctival incision or excision;
c. implantation of a medical device within or around the eye; and/or
d. a corneal incision.

18. The method of claim 16 or 17, wherein the ocular surgery is micro-invasive glaucoma surgery, glaucoma filtration surgery, cataract surgery, retinal detachment repair surgery, strabismus surgery, vitrectomy, pterygium removal, an excisional biopsy, trauma reconstruction, or implantation of a stent, valve, implant or shunt within or around the eye.

19. The method of any one of claims 7-18, wherein the COX2 Ser516 acetylating agent and the cPLA2 agonist are administered sequentially, in either order.

20. The method of any one of claims 7-18, wherein the COX2 Ser516 acetylating agent and the cPLA2 agonist are administered simultaneously.

21. The method of any one of claims 7-18, wherein the COX2 Ser516 acetylating agent and the cPLA2 agonist are formulated in the same composition.

22. The method of any one of claims 7-21, wherein the COX2 Ser516 acetylating agent and the cPLA2 agonist are administered locally.

23. The method of any one of claims 7-22, wherein the COX2 Ser516 acetylating agent is ASA or a 2-acetoxyphenyl alkyl sulfide.

24. The method of claim 23, wherein the 2-acetoxyphenyl alkyl sulfide is APHS.

25. The method of any one of claims 7-24, wherein the cPLA2 agonist is gentamicin, tobramycin, mastoparan, PLAP, tetrahydrofurandiol or melittin.

26. A method of resolving ocular inflammation in a subject, comprising locally administering a COX2 Ser516 acetylating agent to the subject.

27. A method of decreasing ocular fibrosis and/or scarring in a subject, comprising locally administering a COX2 Ser516 acetylating agent to the subject.

28. A method of decreasing ocular collagen contraction and/or remodelling in a subject, comprising locally administering a COX2 Ser516 acetylating agent to the subject.

29. A method of decreasing ocular fibroblast cellular proliferation in a subject, comprising locally administering a COX2 Ser516 acetylating agent to the subject.

30. The method of claim 29, wherein the fibroblast is a myofibroblast.

31. The method of claim 29 or 30, wherein the fibroblast is a human Tenon's capsule fibroblast (HTCF).

32. The method of any one of claims 26-31, wherein the ocular inflammation is inflammation of the conjunctiva.

33. The method of claim 32, wherein the inflammation of the conjunctiva is caused by conjunctivitis.

34. The method of claim 33, wherein the conjunctivitis is allergic conjunctivitis.

35. The method of any one of claim 26-31, wherein the COX2 Ser516 acetylating agent is administered before, during and/or after ocular surgery.

36. The method of claim 35, wherein the ocular surgery comprises one or more of:
a. manipulation of the conjunctiva and/or Tenons;
b. a conjunctival incision or excision;
c. implantation of a medical device within or around the eye; and/or
d. a corneal incision.

37. The method of claim 35 or 36, wherein the ocular surgery is micro-invasive glaucoma surgery, glaucoma filtration surgery, cataract surgery, retinal detachment repair surgery, strabismus surgery, vitrectomy, pterygium removal, an excisional biopsy, trauma reconstruction, or implantation of a stent, valve, implant or shunt within or around the eye.

38. The method of any one of claims 26-37, wherein the COX2 Ser516 acetylating agent is ASA or a 2-acetoxyphenyl alkyl sulfide.

39. The method of claim 38, wherein the 2-acetoxyphenyl alkyl sulfide is APHS.

40. The method of any one of claims 7-39, wherein the administering decreases ocular fibroblast prostaglandin production;
increases ocular fibroblast 5-hydroxyeicosatetraenoic acid (5-HETE), 15-hydroxyeicosatetraenoic acid (15-HETE) and/or 17-hydroxy-docosahexaenoic acid (17-OHDHA) production;
decreases ocular fibroblast metabolic activity;
decreases ocular fibroblast collagen production;
decreases ocular fibroblast alpha smooth muscle actin (αSMA) expression;
decreases ocular fibroblast marker of proliferation Ki-67 (Ki-67) expression;
decreases ocular fibroblast matrix metalloproteinase (MMP) expression;
increases ocular fibroblast peroxisome proliferator-activated receptor gamma (PPARγ) expression;
decreases ocular fibroblast SMAD family member 2/3 (SMAD2/3) expression; and/or
decreases ocular fibroblast SMAD2/3 phosphorlation.
